(19) United States
(12) Patent Application Publication
(10) Pub. No.: US 2021/0353585 A1
(43) Pub. Date: Nov. 18, 2021

(54) METHODS AND COMPOSITIONS FOR TREATING LAMINOPATHIES AFFECTING SKELETAL OR CARDIAC MUSCLE

(71) Applicant: CORNELL UNIVERSITY, Ithaca, NY (US)

(72) Inventors: Jan LAMMERDING, Ithaca, NY (US); Tyler J. KIRBY, Brooktondale, NY (US); Gregory R. FEDORCHAK, Ithaca, NY (US); Ashley J. EARLE, Dryden, NY (US)

(21) Appl. No.: 17/274,131

(22) PCT Filed: Sep. 6, 2019
(86) PCT No.: PCT/US19/50044

§ 371 Date: Mar. 5, 2021

Related U.S. Application Data
(60) Provisional application No. 62/728,472, filed on Sep. 7, 2018.

(51) Int. Cl. A61K 31/337; A61K 45/06; A61K 31/427

(52) U.S. Cl. CPC A61K 31/337; A61K 31/427; A61K 45/06

(57) ABSTRACT

The present application relates to methods of treating a laminopathy affecting skeletal or cardiac muscle in a subject. The methods involve selecting a subject who has a laminopathy affecting skeletal or cardiac muscle. In some embodiments, the methods involve administering, to the selected subject, an inhibitor of a protein associated with a DNA damage response (DDR) pathway to treat the laminopathy affecting skeletal or cardiac muscle in the subject. In other embodiments, the methods involve administering, to the selected subject, a microtubule stabilizing agent and a Linker of Nucleoskeleton and Cytoskeleton (LINC) complex disruptor to treat the laminopathy affecting skeletal or cardiac muscle in the subject. Also disclosed are pharmaceutical compositions comprising an inhibitor of a protein associated with a DNA damage response (DDR) pathway, a microtubule stabilizing agent, and/or a Linker of Nucleoskeleton and Cytoskeleton (LINC) complex disruptor.

This application claims the priority benefit of U.S. Provisional Patent Application Ser. No. 62/728,742, filed Sep. 7, 2018, which is hereby incorporated by reference in its entirety.

This invention was made with government support under RO1HL082792 and U54 CA210184 awarded by National Institutes of Health; BC150580 awarded by Department of Defense; and CBET-1254846, MCB-1715606, 2013160437, and 2014163403 awarded by National Science Foundation. The government has certain rights in this invention.

### FIELD

Disclosed herein are methods and composition for treating laminopathies affecting skeletal or cardiac muscle in a subject.

### BACKGROUND

Lamins A and C, together with the B-type lamins, are the major components of the nuclear lamina, which line the inner nuclear membrane. Lamins A/C play important roles in providing structural support to the nucleus and connecting the nucleus to the cytoskeleton (de Leeuw et al., “Nuclear Lamins: Thin Filaments with Major Functions,” Trends Cell Biol. 28:3445 (2018)). In addition, they participate in transcriptional regulation, genome organization, and DNA damage and repair (de Leeuw et al., “Nuclear Lamins: Thin Filaments with Major Functions,” Trends Cell Biol. 28(1):34-45 (2018); Worman, H. J., “Cell Signaling Abnormalities in Cardiomyopathy Caused by Lamin A/C Gene Mutations,” Biochem. Soc. Trans. 46(1):37-42 (2018); Naetar et al., “Lamins in the Nuclear Interior—Life Outside the Lamina,” J. Cell Sci. 130(13):2087-2096 (2017); and Davidson et al., “Broken Nuclei-Lamins, Nuclear Mechanics, and Disease,” Trends Cell Biol. 24(4):247-256 (2014)). The majority of the over 450 LMNA mutations identified to date are responsible for autosomal dominant Emery-Dreifuss muscular dystrophy (AD-EDMD) (Maggi, et al., “Skeletal Muscle Laminopathies: A Review of Clinical and Molecular Features,” Cells 5(3) (2016)), characterized by slowly progressive skeletal muscle wasting, contractures of the elbow, neck, and Achilles tendons, a rigid spine, abnormal heart rhythms, heart block, and cardiomyopathy (Emery, A. E., “Emery-Dreifuss Muscular Dystrophy—a 40 Year Retrospective,” Neuromuscul. Disord. 10(4-5):228-32 (2007)). Other LMNA mutations cause congenital muscular dystrophy (LMNA-CMD), a particularly severe form of muscular dystrophy with onset in early childhood (Quijano-Roy et al., “De novo LMNA Mutations cause a New Form of Congenital Muscular Dystrophy,” Annals of Neurology 64:177-186 (2008)), and limb girdle muscular dystrophy, which affects proximal muscles of the hips and shoulders (Muchir et al., “Identification of Mutations in the Gene Encoding Lamins A/C in Autosomal Dominant Limb Girdle Muscular Dystrophy with Atrioventricular Conduction Disturbances (LGMD1B),” Hum. Mol. Genet. 9(9):1453-1459 (2000)). It remains unclear how LMNA mutations result in muscle-specific defects, and the incomplete understanding of the disease pathogenesis presents a major hurdle in the development of effective treatment approaches.

One potential explanation, the ‘mechanical stress’ hypothesis, states that LMNA mutations linked to muscular phenotypes result in structurally impaired nuclei that become damaged in mechanically active tissues, such as cardiac and skeletal muscle (Davidson et al., “Broken Nuclei-Lamins, Nuclear Mechanics, and Disease,” Trends Cell Biol. 24(4):247-256 (2014)). This hypothesis is supported by findings of decreased nuclear stiffness in fibroblasts expressing LMNA mutations linked to striated muscle laminopathies, impaired assembly of mutant lamins in vitro, and anecdotal reports of nuclear envelope damage in skeletal and cardiac muscle cells of individuals with AD-EDMD and LMNA-related dilated cardiomyopathies (de Leeuw et al., “Nuclear Lamins: Thin Filaments with Major Functions,” Trends Cell Biol. 28:34-45 (2018); Davidson et al., “Broken Nuclei-Lamins, Nuclear Mechanics, and Disease,” Trends Cell Biol. 24:247-256 (2014); Zwerger et al., “Myopathic Lamin Mutations Impair Nuclear Stability in Cells and Tissue and Disrupt Nucleo-Cytoskeletal Coupling,” Hum. Mol. Genet. 22:2335-2349 (2013); and Gruenbaum et al., “Lamins: Nuclear Intermediate Filament Proteins with Fundamental Functions in Nuclear Mechanics and Genome Regulation,” Annu. Rev. Biochem. 84:131-164 (2015). However, striated muscle laminopathies involve progressive muscle wasting, and analysis of fixed tissues and cells provides only a snapshot of the development of disease. Thus, the functional relevance of nuclear envelope damage, particularly whether the nuclear envelope damage is a cause or consequence of muscle dysfunction, remains unclear. This incomplete understanding presents a major hurdle in the development of therapeutic strategies, as no effective treatments currently exist for these diseases.

The present application is directed to overcoming these and other deficiencies in the art.

### SUMMARY

A first aspect of the present application relates to a method of treating a laminopathy affecting skeletal or cardiac muscle in a subject. This method involves selecting a subject who has a laminopathy affecting skeletal or cardiac muscle and administering, to the selected subject, an inhibitor of a protein associated with a DNA damage response (DDR) pathway to treat the laminopathy affecting skeletal or cardiac muscle in the subject.

Another aspect of the present application relates to a method of treating a laminopathy affecting skeletal or cardiac muscle in a subject. This method involves selecting a subject who has a laminopathy affecting skeletal or cardiac muscle; administering, to the selected subject, a microtubule stabilizing agent; and administering, to the selected subject, a Linker of Nucleoskeleton and Cytoskeleton (LINC) complex disruptor before, after, or during said administering the microtubule stabilizing agent to treat the laminopathy affecting skeletal or cardiac muscle in the subject.

Yet another aspect of the present application relates to a pharmaceutical composition comprising an inhibitor of a protein associated with a DNA damage response (DDR) pathway and a microtubule stabilizing agent.

A further aspect of the present application relates to a pharmaceutical composition comprising a Linker of Nucleoskeleton and Cytoskeleton (LINC) complex disruptor and a microtubule stabilizing agent.

To better understand the mechanistic link between nuclear envelope damage and muscle dysfunction, three laminopathy mouse models with varying degrees of disease severity, along with a recently developed long-term in vitro muscle differentiation platform (Pimentel et al., “In Vitro Differentiation of Mature Myofibers for Live Imaging,” J. Vis. Exp. 119 (2017), which is hereby incorporated by reference in its entirety), were employed. Using high resolution time lapse microscopy, myonuclear shape and integrity was tracked over time and structural and functional changes during the differentiation of primary mouse myoblasts into multinucleated myotubes and subsequent formation of mature, contractile myofibers were recorded (FIGS. 1A-1B). By combining these models with novel, fluorescent nuclear damage reporters (Denais et al., “Nuclear Envelope Rupture and Repair During Cancer Cell Migration,” Science 352:353-358 (2016), which is hereby incorporated by reference in its entirety), both in vitro and in vivo, unprecedented systematic and detailed temporal and mechanistic information of disease progression in mouse models of laminopathies was obtained, and key findings in skeletal muscle biopsies from humans with LMNA muscular dystrophy were corroborated.

The experimental results presented infra demonstrate that myonuclei in Lmna mutant muscle cells exhibited progressive nuclear envelope damage in vitro and in vivo, including extensive chromatin protrusions into the cytoplasm and transient nuclear envelope rupture. Intriguingly, nuclear envelope rupture was associated with progressive DNA damage and DNA damage response activation. Inducing DNA damage in wild-type muscle cells was sufficient to provoke defects in cell viability similar to those observed in the lamin A/C-deficient cells. In addition, preventing nuclear envelope rupture by LINC complex disruption was sufficient to reduce DNA damage and rescue myofiber viability and contractility in lamin A/C-deficient cells. Together, these findings indicate a causative role of nuclear envelope rupture and DNA damage in progressive muscle decline and provide a novel explanation for how lamin mutations lead to muscle weakness and wasting in AD-EDMD.

### BRIEF DESCRIPTION OF THE DRAWINGS

FIGS. 1A-1F demonstrate that in vitro differentiated primary myoblasts from homozygous Lmna KO, Lmna N195K, and Lmna H222P mice recapitulate disease severity. FIG. 1A is a graphical representation of the three Lmna mutant models used in the study, indicating the published 50% mortality rates of Lmna KO, Lmna N195K, and Lmna H222P mice, as well as wild-type (Lmna WT) controls. Shading represents the onset of disease symptoms in the mouse models. FIG. 1B is a schematic for the stages of differentiation from primary myoblasts into mature myofibers in the in vitro system. FIG. 1C shows representative images of Lmna WT, Lmna KO, Lmna N195K, and Lmna H222P primary skeletal muscle cells at days 0, 5, and 10 of differentiation. Scale bar: 100 μm. FIG. 1D shows the quantification of cell viability using MTT assay at days 5, 10 of differentiation. n=3-6 independent cell lines for each genotype. **, p<0.01 vs. Lmna WT; *, p<0.05 vs. Lmna WT. FIG. 1E is a representative image of cleaved caspase-3 immunofluorescence in Lmna WT and Lmna KO myofibers at day 10 of differentiation. Scale bar: 20 μm FIG. 1F shows the quantification of cleaved caspase-3 relative to myosin heavy chain immunofluorescence area in Lmna WT, Lmna KO, Lmna N195K, and Lmna H222P myofibers after 10 days of differentiation ***, p<0.001 vs. Lmna WT; *, p<0.05 vs. Lmna WT. n=3 independent cell lines for each genotype.

FIGS. 2A-2F demonstrate that Lmna display varying degrees of muscular dystrophy. FIG. 1A shows the quantification of the average myofiber cross-sectional area of Lmna WT and Lmna KO mice. FIG. 2B shows the relative frequency of myofiber cross-sectional area in Lmna WT and Lmna KO mice. ***, p<0.001 vs. Lmna WT; n=8-10 animals per genotype. FIG. 2C shows the quantification of the average myofiber cross-sectional area of Lmna WT and Lmna N195K mice. FIG. 2D shows the relative frequency of myofiber cross-sectional area in Lmna WT and Lmna N195K mice. **, p<0.01 vs. Lmna WT; n=11-12 animals per genotype. FIG. 2E shows the quantification of the average myofiber cross-sectional area of Lmna WT and Lmna H222P mice. FIG. 2F shows the relative frequency of myofiber cross-sectional area in Lmna WT and Lmna H222P mice. n=3-4 animals per genotype.

FIG. 3 demonstrates that in vitro differentiation results in mature myofibers. Representative image of a striated myofiber containing a peripheral nucleus at day 10 of differentiation. Scale bar: 5 μm.

FIGS. 4A-4B demonstrates that Lmna KO and Lmna N195K have reduced contractility and experience nuclear loss. FIG. 4A shows the quantification of myofiber contraction at day 10 of differentiation. Fibers were assigned contraction scores from 0 (worst) to 4 (best) based on the percentage of cells that were visually contracting. **, p<0.01 vs. Lmna WT, *, p<0.05 vs. Lmna WT; n=3-6 independent cell lines for each genotype. FIG. 4B shows the quantification of the change in nuclear number between day 5 and day 10 of differentiation. ***, p<0.001 vs. Lmna WT *, p<0.05 vs. Lmna WT; n=3-6 independent experiments from 3 independent cell lines per genotype.

FIGS. 5A-5G demonstrate that Lmna mutant muscle cells display defects in nuclear stability. FIG. 5A shows representative images of Lmna WT and Lmna KO nuclei deforming in a microfluidic micropipette aspiration device. Scale bar: 10 μm. FIG. 5B shows the measurement for nuclear deformation at 5 second intervals for Lmna WT, Lmna KO, Lmna N195K, and Lmna H222P myoblasts during 60 seconds of aspiration. FIG. 5C shows the quantification of the nuclear deformation after 60 seconds of aspiration. n=41-67 nuclei per genotype from 3 independent experiments. ***, p<0.001 vs. Lmna WT. FIG. 5D shows the results of a microharpoon assay used to measure nuclear deformability (ΔL/L₀) in myofibers, showing representative images before and at the end of perinuclear cytoskeletal strain application with a microneedle (dashed line). Scale bar: 15 μm. FIG. 5E shows the quantification of nuclear strain induced by microharpoon assay in Lmna WT and Lmna KO myotubes at day 5 of differentiation. n=19-22 nuclei per genotype from 3 independent experiments. ***, p<0.001 vs. Lmna WT myotubes. FIG. 5F shows a representative image of nuclear morphology in Lmna WT, Lmna KO, Lmna N195K, and Lmna H222P myotubes after 5 days of differentiation. Scale bar: 20 μm. FIG. 5G shows the nuclear aspect ratio (length/width) in Lmna WT, Lmna KO, Lmna N195K, and Lmna H222P myotubes after 5 days of differentiation. n=3-4 independent cell lines per genotype with >100 nuclei counted per image. **, p<0.01 vs. Lmna WT.

FIGS. 6A-6D show micropipette aspiration analysis of Lmna mutant myoblasts and Lmna KO myoblasts ectopically expressing lamin A. FIG. 6A is a natural log transformation and plot of the micropipette aspiration data shown in FIG. 5B. The log-log data fits a linear regression model, in which all three Lmna mutants were significantly different (p<0.001) from the wild-type controls. The slopes of the log-log data were not significantly different between the samples. A multilevel model including day-to-day variability confirmed that all three Lmna mutants were significantly different from the wild-type controls (p<0.0001 for Lmna KO and Lmna N195K; p<0.001 for Lmna H222P), although the statistical significance for the Lmna H222P myoblasts was lost when including additional variance components. FIG. 6B shows representative immunofluorescence images of lamin A expression in Lmna WT, Lmna KO and Lmna KO cells ectopically expressing lamin A (Lmna KO+lamin A). FIG. 6C shows the measurement for nuclear deformation at 5 second intervals for Lmna WT, Lmna KO, and Lmna KO+Lamin A myoblasts during 60 seconds of aspiration. FIG. 6D shows the quantification of the nuclear deformation after 60 seconds of aspiration, showing that ectopic expression of lamin A significantly improves nuclear stiffness in Lmna KO myoblasts. n=41-67 nuclei per genotype from 3 independent experiments. n=62-73 nuclei per genotype from 3 independent experiments. ***, p<0.001 vs Lmna WT cells. †, p<0.01 vs. Lmna KO cells.

FIG. 7 shows that Mcbc myoblasts have normal nuclear stiffness. Measurement for nuclear deformation at 5 second intervals for Lmna WT and mdx myoblasts during 60 seconds of aspiration. n=35-67 cells per condition.

FIGS. 8A-8F demonstrate that Lmna mutant myonuclei develop chromatin protrusions during differentiation. FIG. 8A shows representative images of chromatin protrusions observed in Lmna KO myofibers after 10 days of differentiation. Yellow arrowheads indicate the end of the protrusion; the white arrowheads indicate a thin chromatin tether protruding from the nucleus. Scale bar: 10 μm. FIG. 8B shows the quantification of the percentage of myonuclei containing chromatin protrusion at days 5 and 10 of differentiation in Lmna WT, Lmna KO, Lmna KO+Lamin A, Lmna N195K and Lmna H222P cell lines. Data from n=3 independent experiments with 62-73 nuclei per genotype. ***, p<0.001 vs. Lmna WT cells. \, p<0.01 vs. Lmna KO. FIG. 8C shows representative images of isolated single muscle fibers from Lmna WT and Lmna KO mice labeled for lamin B1 (green) and DNA (magenta). Arrowheads indicate the presence of chromatin protrusions in Lmna KO muscle fiber. Scale bar: 20 μm. FIG. 8D shows the quantification of the percentage of myonuclei with chromatin protrusion in isolated muscle fibers from Lmna WT and Lmna KO mice. Left, data based on analysis of total muscle fiber. Right, analysis for nuclei located at the myotendinous junctions compared to those within the body of the fiber. n=8-11 mice per genotype, with 5 single fibers imaged per animal. ***, p<0.001 vs. Lmna WT. \, p<0.01 vs. nuclei in the muscle body. FIG. 8E shows the generation of hybrid myofibers. The top panel is a schematic of the generation of hybrid myofibers containing nuclei from both Lmna WT and Lmna KO cell lines. The bottom panel shows corresponding representative images. Final hybrid fibers contained ˜80% Lmna WT nuclei and 20% Lmna KO nuclei. Arrowheads denote Lmna KO nucleus with a chromatin protrusion residing within the same myofiber as a Lmna WT nucleus. FIG. 8F shows the quantification of the number of chromatin protrusions from Lmna WT and Lmna KO contained within isogenic myofibers (control) or hybrid myofibers containing 80% Lmna WT and 20% Lmna KO nuclei. n=3 independent experiments, in which 91-163 nuclei were quantified per experiment.

FIGS. 9A-9B demonstrate that chromatin protrusions are surrounded by nuclear membranes containing emerin, with disturbed localization of nesprin-1 and nuclear pores. FIG. 9A shows representative immunofluorescence images for nesprin-1 and emerin in Lmna WT, Lmna KO, Lmna N195K and Lmna H222P myofibers at day 5 of differentiation. Blue and yellow arrows denote chromatin protrusions that are enriched with nesprin-1 and emerin, respectively. Scale bar: 20 μm. FIG. 9B shows a representative image of immunofluorescence detection of nuclear pore complexes (NPC) in Lmna KO myofibers at day 10 of differentiation.

Scale bar: 10 μm.

FIGS. 10A-10E demonstrate that Lmna mutant myonuclei undergo nuclear envelope rupture in vitro and in vivo. FIG. 10A shows a representative time-lapse image sequence of nuclear envelope rupture in Lmna KO myonuclei. Red arrowheads mark two nuclei that undergo nuclear envelope rupture, visibly by transient loss of NLS-GFP from the nucleus.

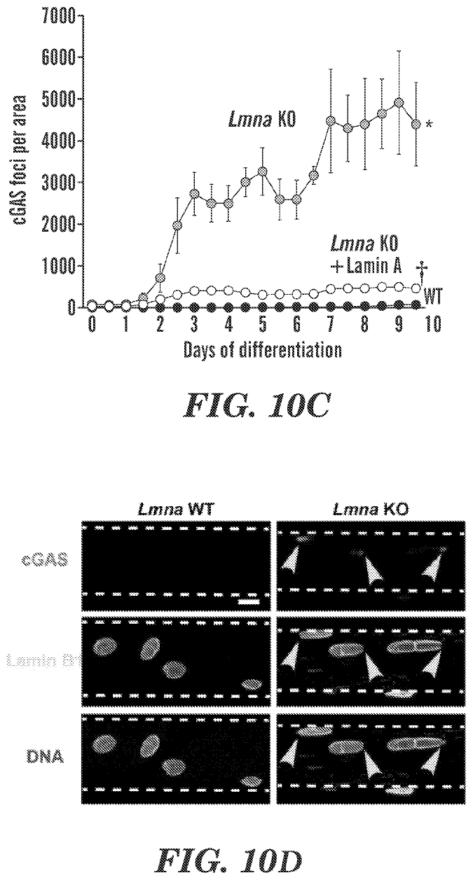

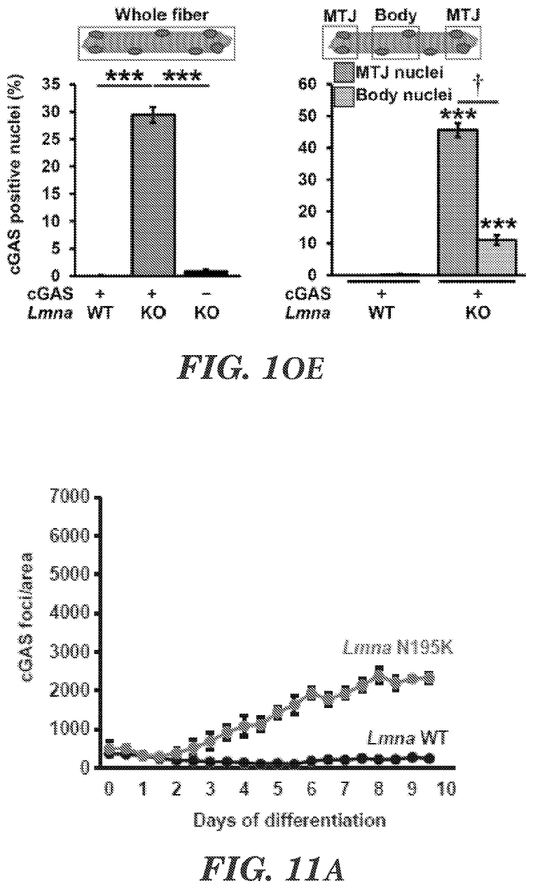

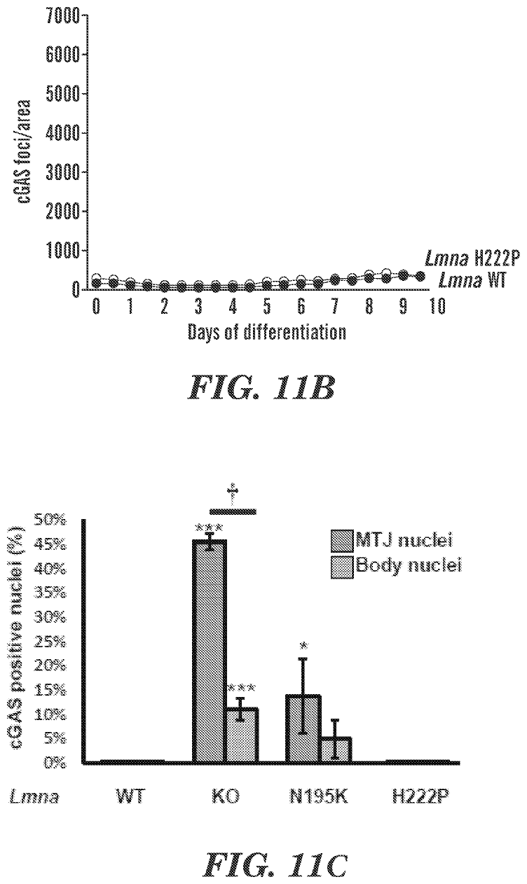

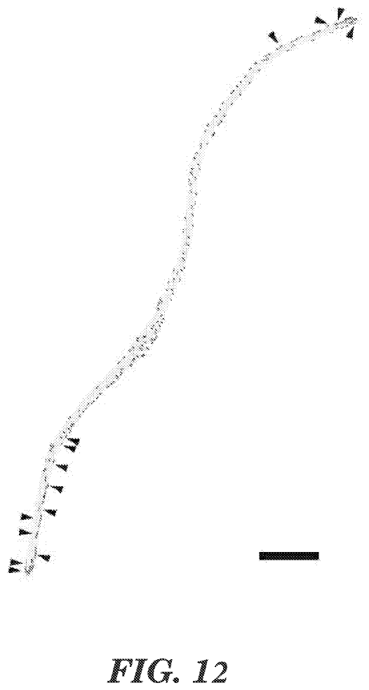

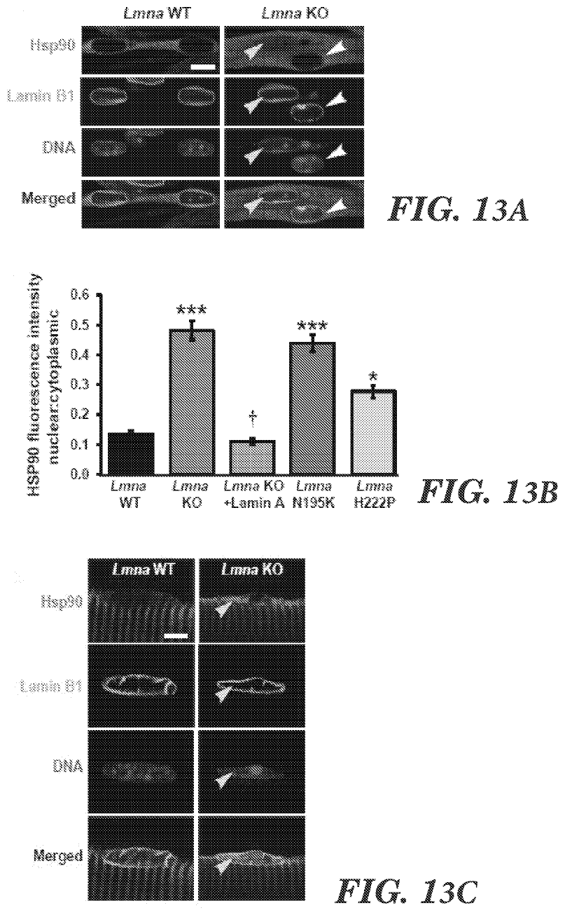

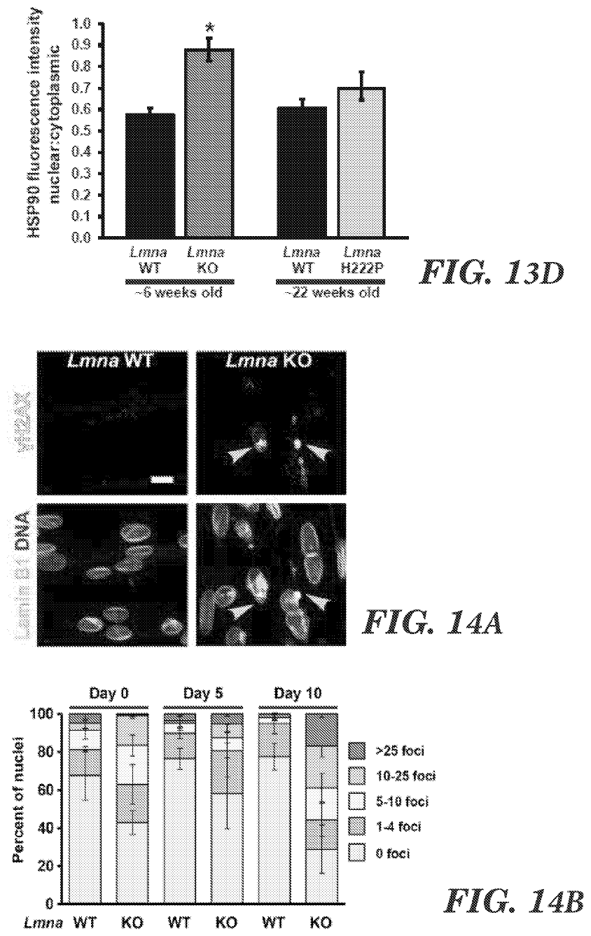

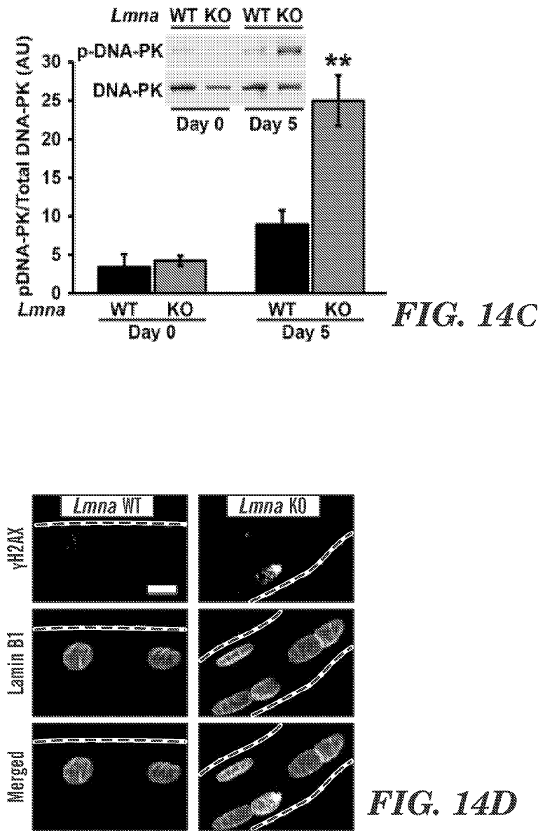

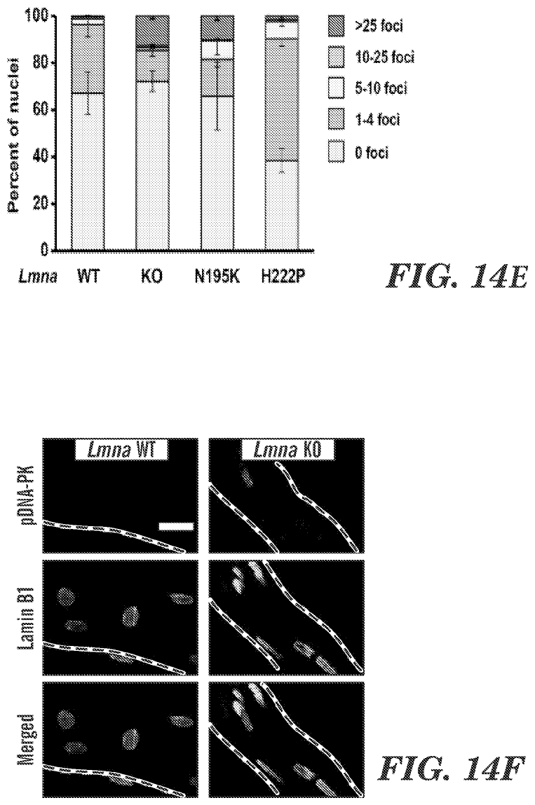

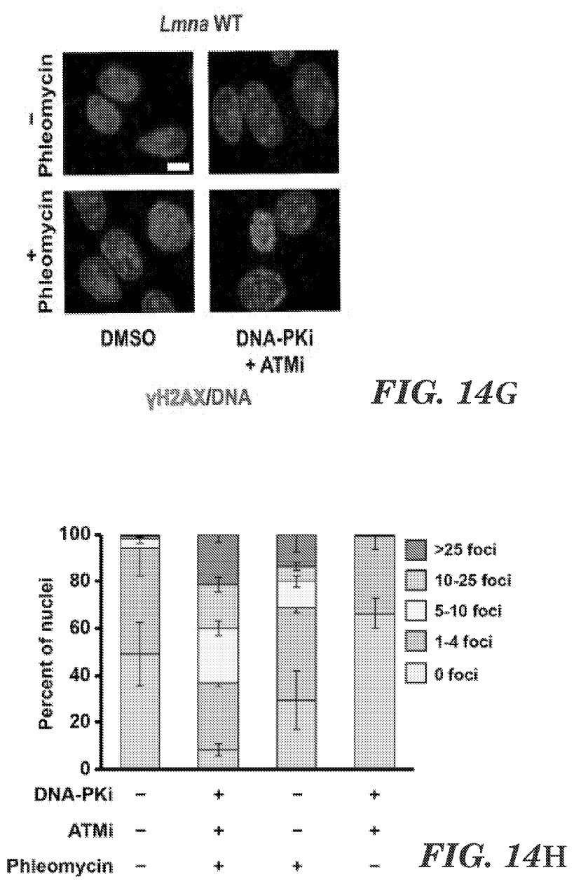

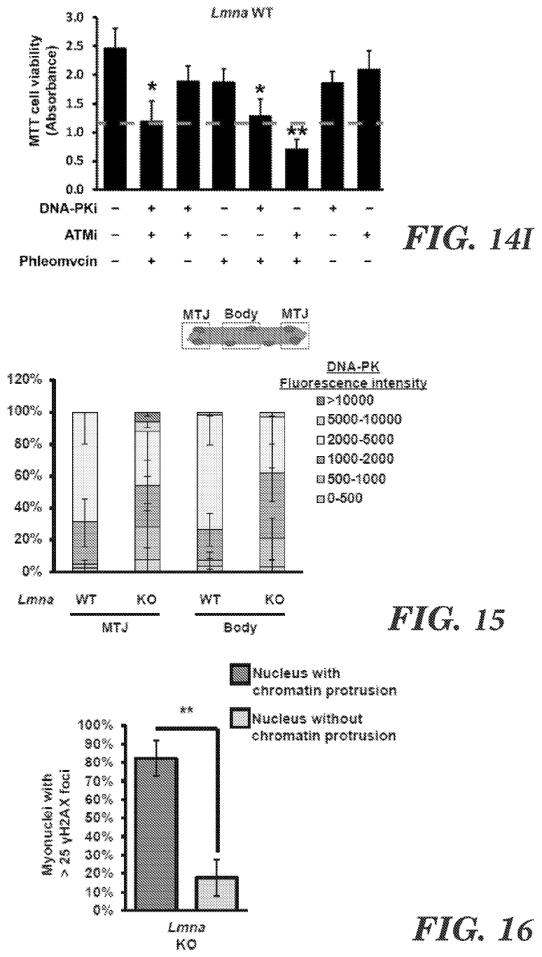

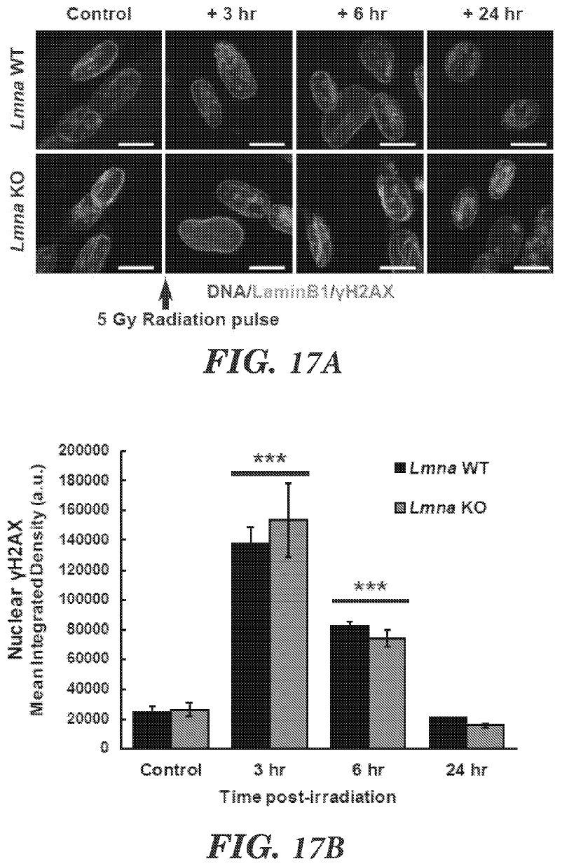

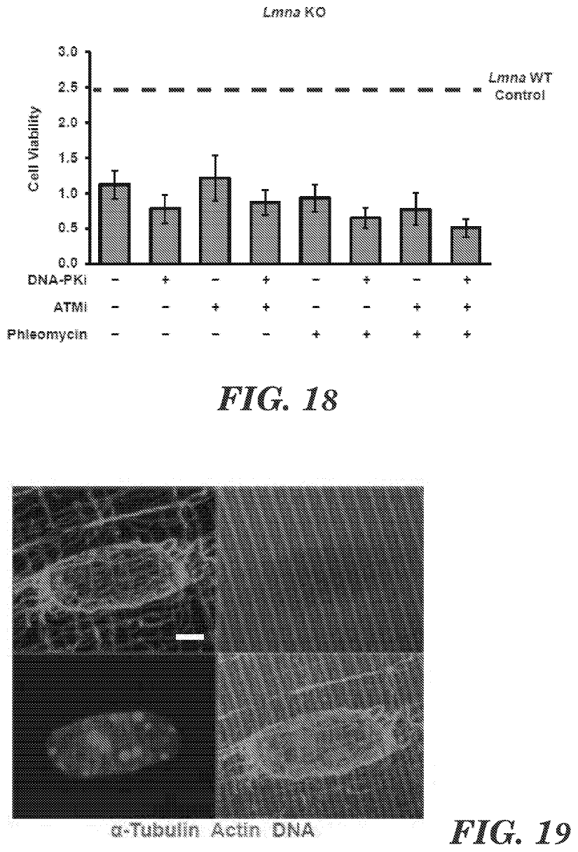

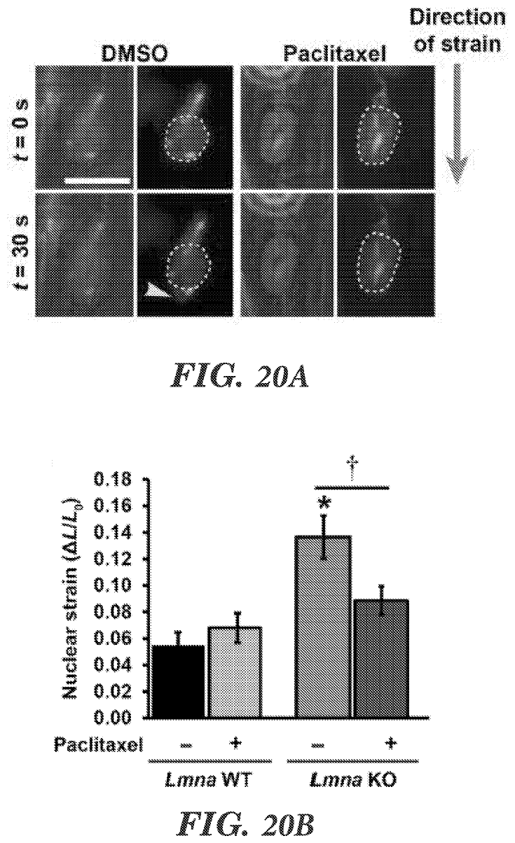

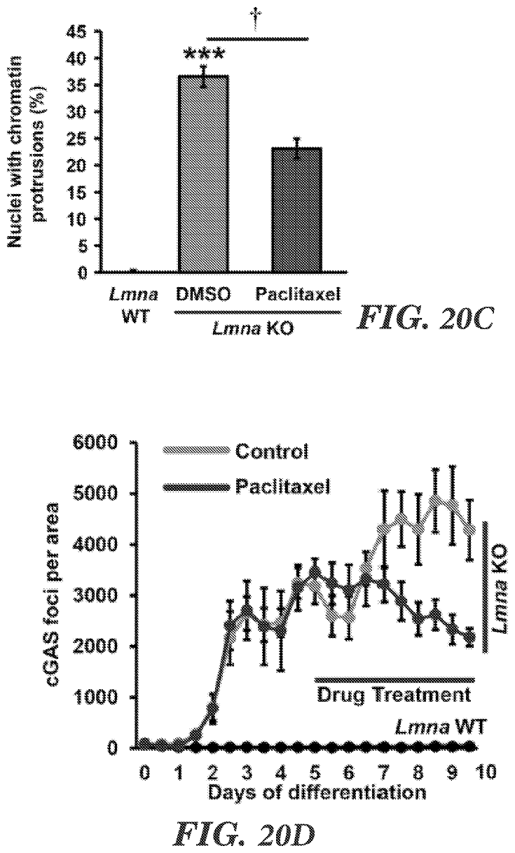

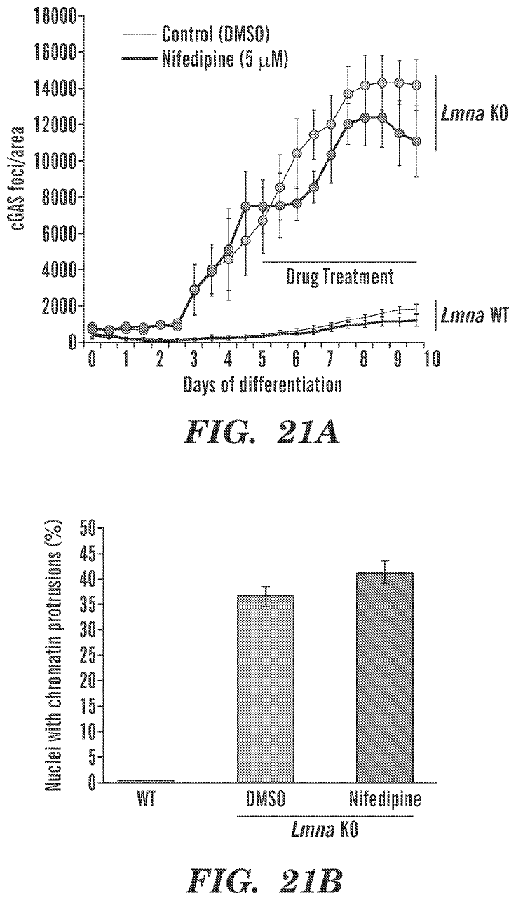

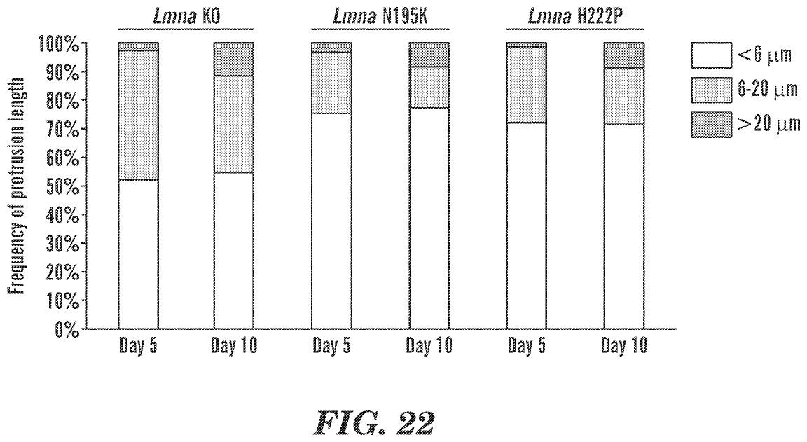

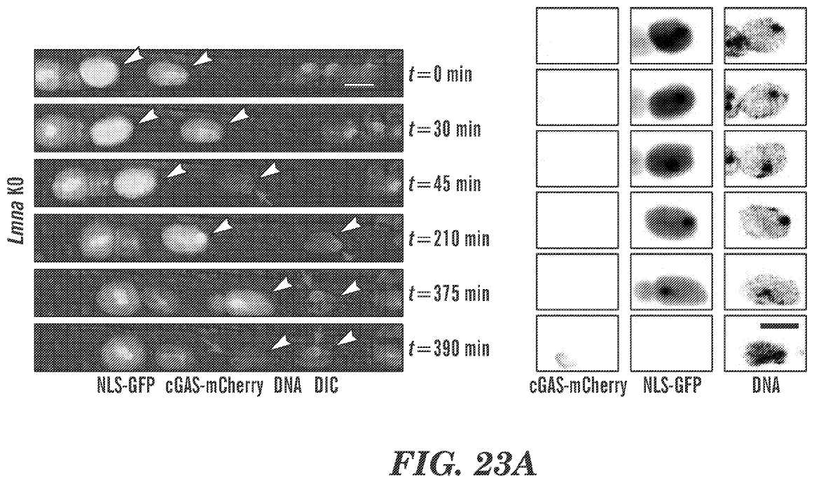

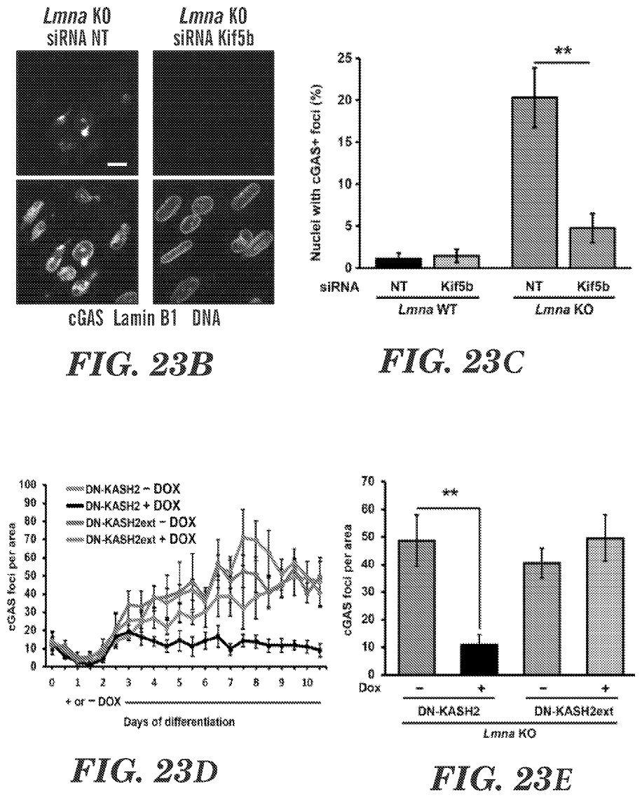

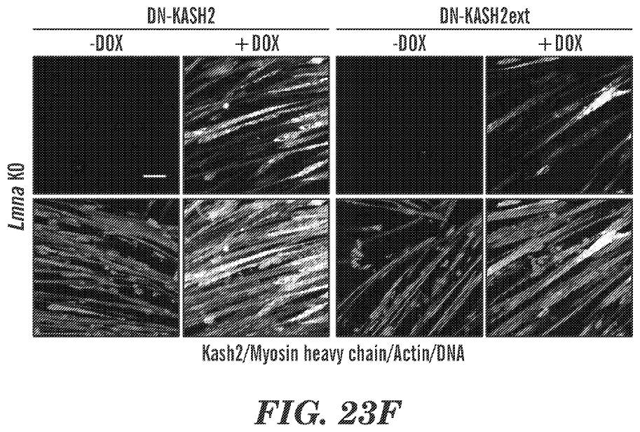

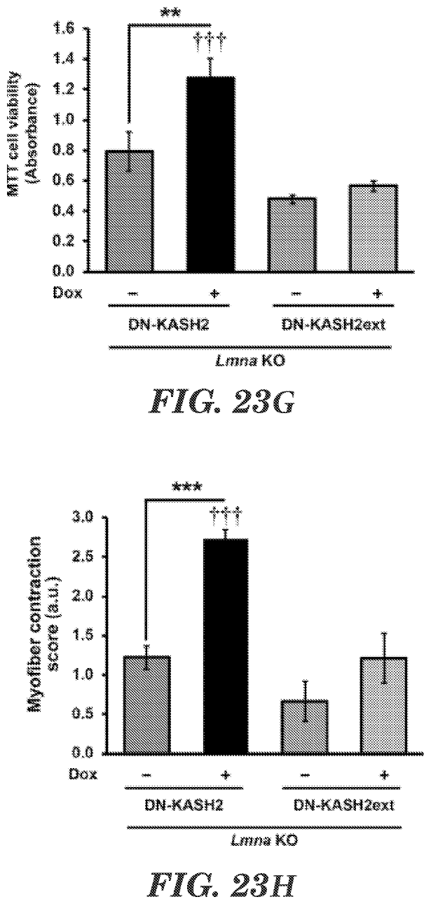

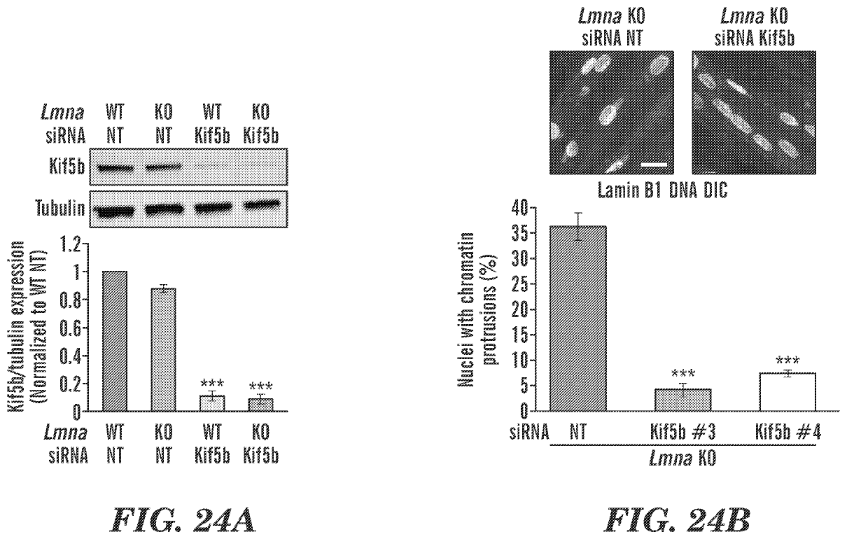

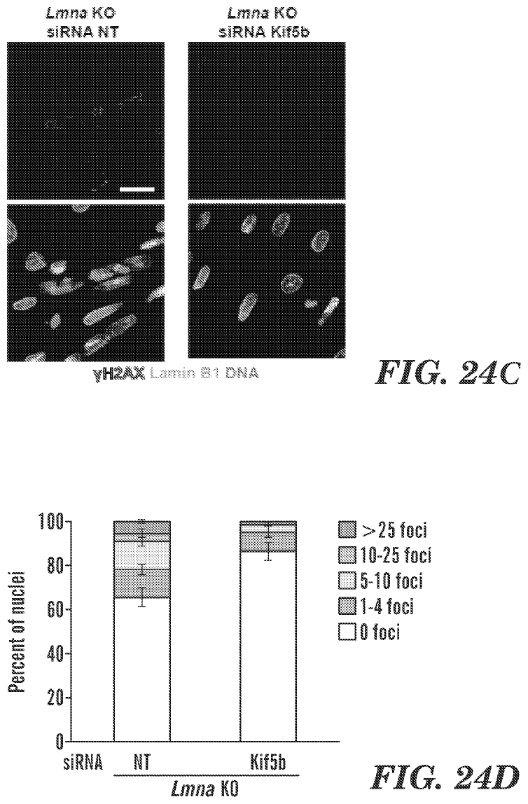

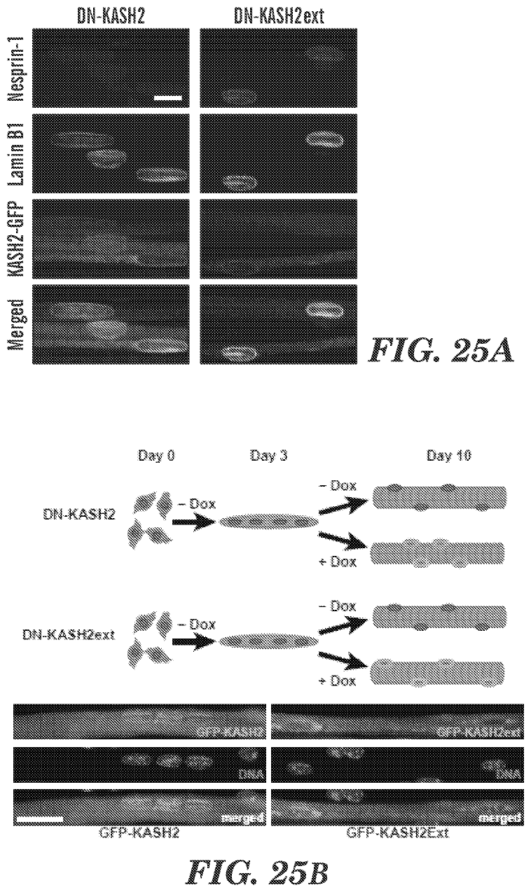

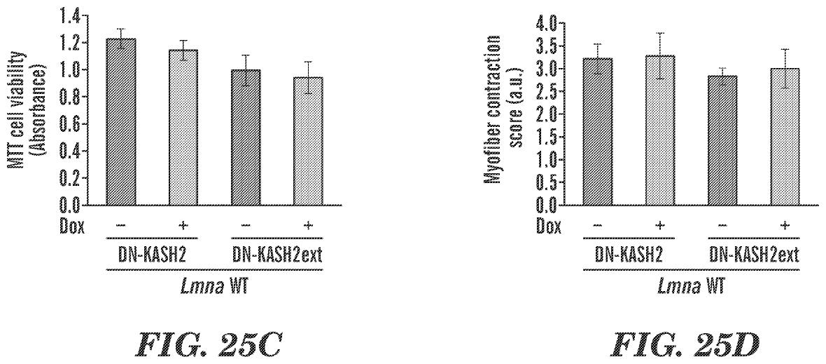

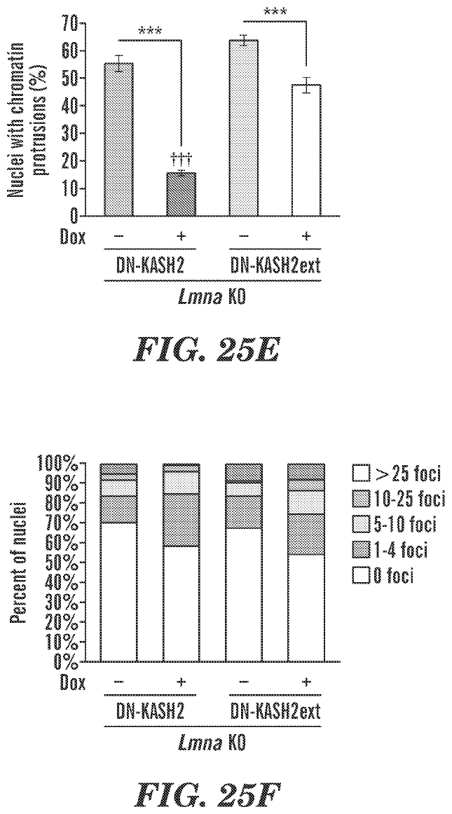

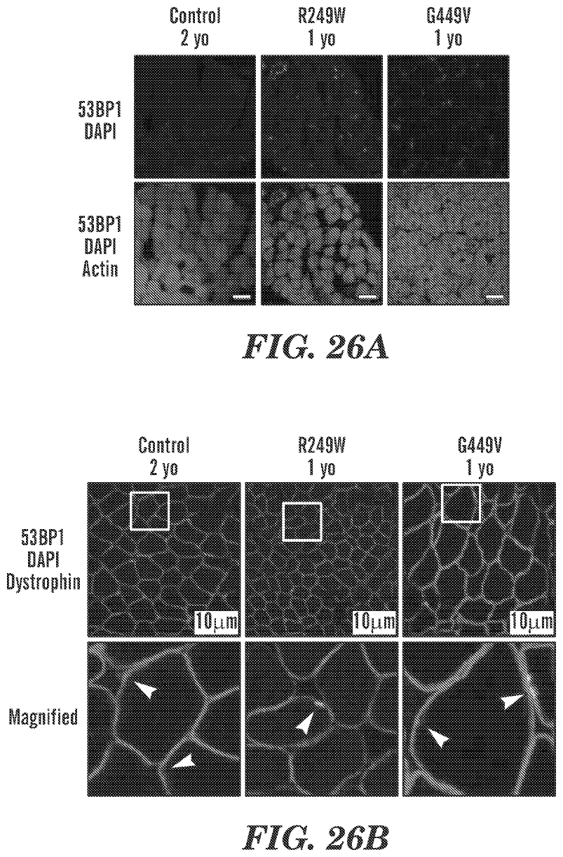

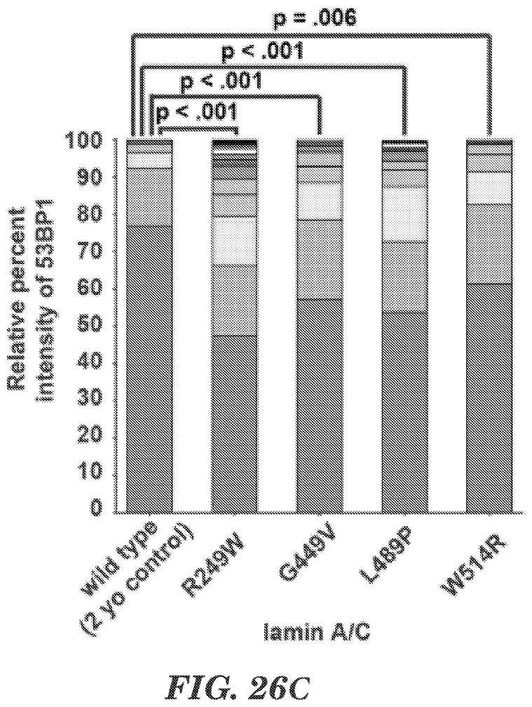

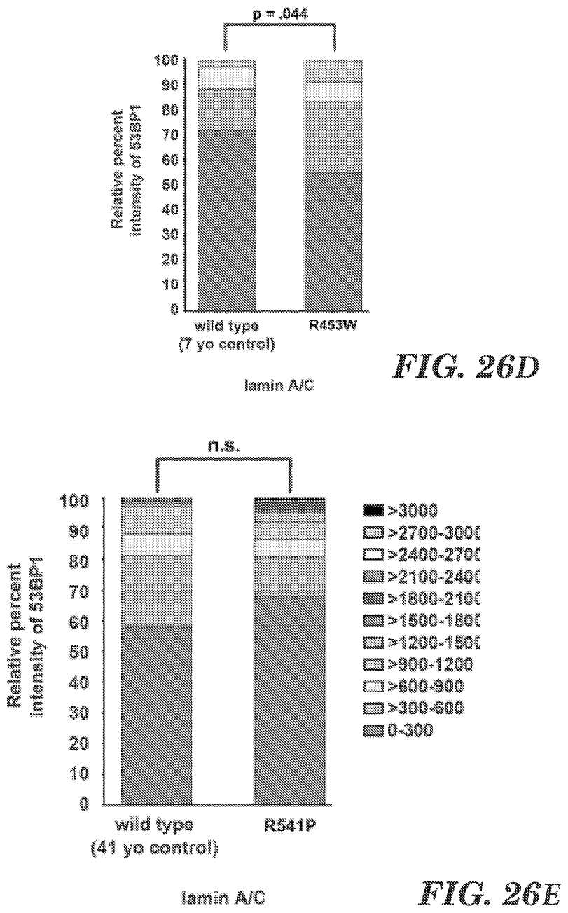

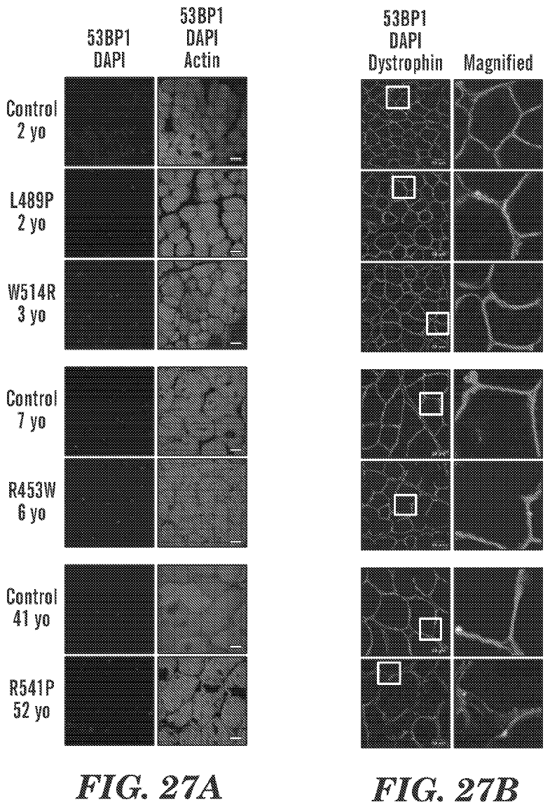

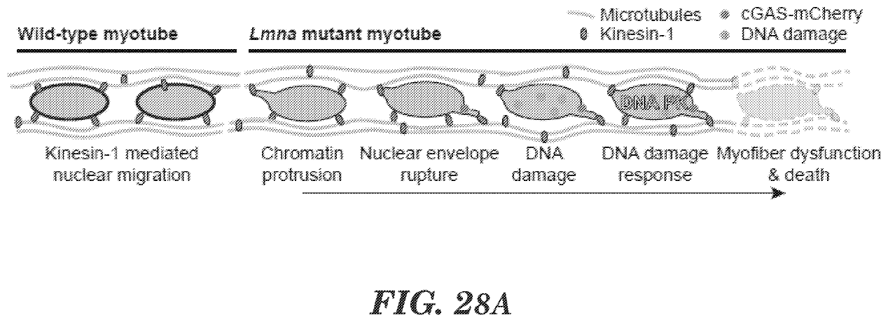

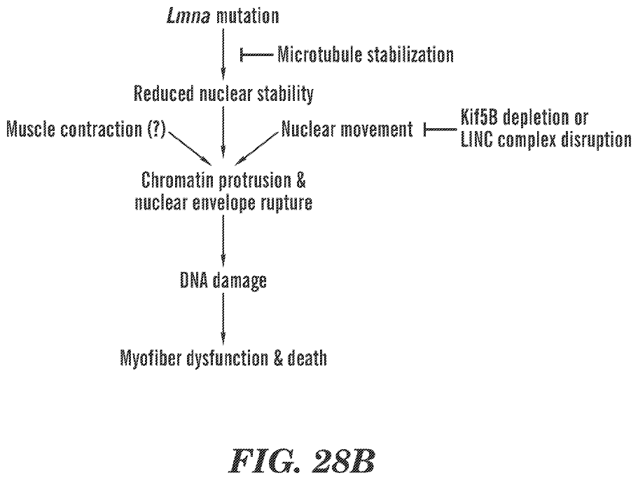

Scale bar: 50 μm for all images. FIG. 10B shows representative images of the accumulation of cGAS-mCherry at the sites of nuclear envelope rupture in Lmna KO myonuclei at day 5 of differentiation. Scale bar: 20 μm. FIG. 10C shows the quantification of cGAS-mCherry foci formation per field of view during myofiber differentiation in Lmna WT, Lmna KO, and Lmna KO cells expressing ectopic lamin A, expressed. n=3 independent experiments. *, p<0.05 vs. Lmna WT. \, p<0.01 vs. Lmna KO. FIG. 10D shows representative maximum intensity projection images of single muscle fibers from Lmna WT and Lmna KO mice expressing a cGAS-tdTomato nuclear envelope rupture reporter, showing accumulation of cGAS-tdTomato at the site of nuclear nuclear envelope in Lmna KO muscle fibers. Scale bar: 10 μm. FIG. 10E shows the quantification of the percentage of myonuclei positive for cGAS-tdTomato foci in isolated muscle fibers from Lmna WT and Lmna KO mice expressing the cGAS-tdTomato transgene (cGAS+) or non-expressing littermates (cGAS−). The latter served as control for potential differences in autofluorescence. Analysis performed for whole fiber (left) and by classification of nuclei located at the myotendinous junctions or within the body of the fiber (right). n=5-8 mice per genotype, with 5 fibers per animal. ***, p<0.001 vs. Lmna WT. \, p<0.01 vs. nuclei in the muscle body.

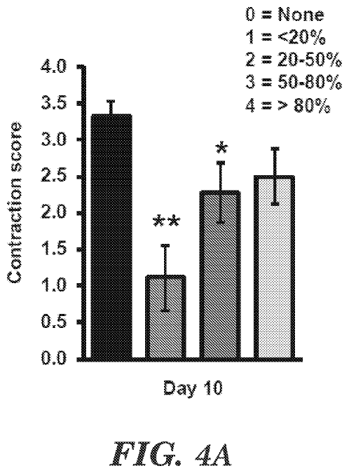

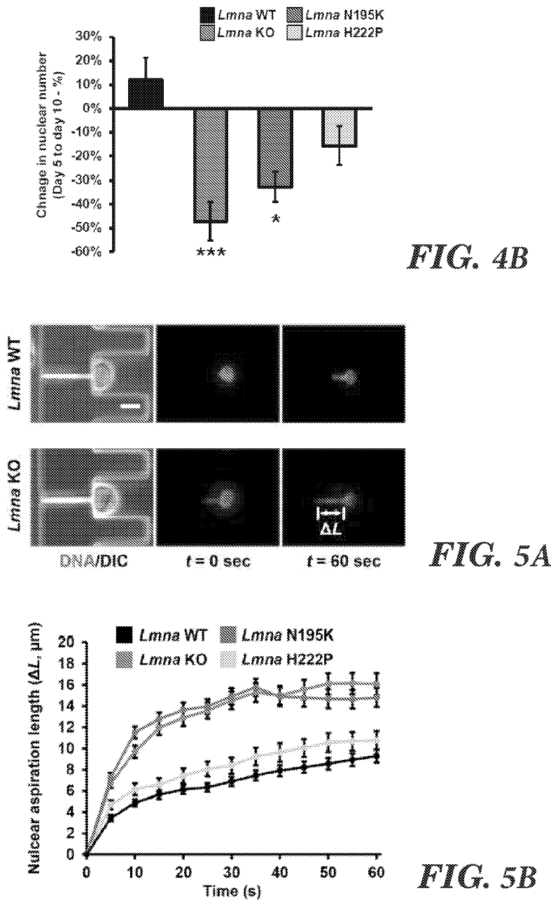

FIGS. 11A-11C demonstrate that nuclear envelope rupture is increased in Lmna N195K myofibers in vitro and in vivo. FIGS. 11A-11B show the quantification of cGAS-mCherry nuclear envelope rupture reporter foci formation during 10 myofiber differentiation in Lmna N195K (FIG. 11A), Lmna H222P (FIG. 11B), and Lmna WT cells. FIG. 11C shows the quantification of the percentage of myonuclei positive for cGAS-tdTomato foci in isolated muscle fibers from Lmna WT, Lmna KO, Lmna N195K and Lmna H222P mice expressing the cGAS-tdTomato transgene. Analysis performed for whole fiber (left) and by classification of nuclei located at the myotendinous junctions or within the body of the fiber (right). Data for Lmna WT and Lmna KO reproduced from FIG. 4E for comparison. n=5-8 mice per genotype, with 5 fibers per animal. ***, p<0.001 vs. Lmna WT. \, p<0.01 vs. nuclei in the muscle body, *, p<0.05 vs. Lmna WT.

FIG. 12 demonstrates that nuclear envelope rupture in Lmna KO muscle fibers is increased at myotendinous junctions. Representative image of a single isolated muscle fiber demonstrating the enrichment of cGAS+nuclei at the myotendinous junctions. Scale bar: 200 μm.

FIGS. 13A-13D demonstrate that Lmna mutant myonuclei have increased presence of Hsp90 in vitro and in vivo. FIG. 13A is a representative image of nuclear localization of a large cytosolic protein, Hsp90, inside Lmna KO nuclei in myofiber differentiated for 10 days. White arrow indicates a nucleus with no observable chromatin defect and little Hsp90 nuclear accumulation, while the yellow arrow marks a nucleus with a chromatin protrusion and increased nuclear Hsp90 accumulation. Scale bar: 10 μm. FIG. 13B shows the quantification of the fluorescence intensity of nuclear Hsp90 levels for Lmna WT, Lmna KO, Lmna KO+Lamin A, Lmna N195K, and Lmna H222P myofibers in vitro. For each nucleus, the nuclear fluorescence intensity was normalized to the cytosolic intensity immediately adjacent to each nucleus. n=25-56 nuclei per genotype from 3 independent experiments. ***, p<0.001 vs. Lmna WT (p<0.001). FIG. 13C is a representative image of Hsp90 nuclear localization in myonuclei from Lmna WT and Lmna KO mice. Scale bar: 10 μm. FIG. 13D shows the quantification of the fluorescence intensity of nuclear HSP90 levels for Lmna WT, Lmna KO, and Lmna H222P isolated single fibers. For each nucleus, the nuclear fluorescence intensity was normalized to the cytosolic intensity immediately beside each nucleus. n=25-56 nuclei per genotype from 3 independent experiments. ***, p<0.001 vs. Lmna WT.

FIGS. 14A-14I demonstrate that Lmna KO mice have increased DNA damage in myonuclei in vitro and in vivo. FIG. 14A shows representative images of γH2AX foci, a marker of a double-stranded DNA break, in Lmna KO myonuclei. Arrowheads indicated γH2AX foci at the sites of chromatin protrusions. Scale bar: 10 μm. FIG. 14B shows the quantification of the extent of DNA damage based on the number of γH2AX foci per nucleus during myofiber differentiation. Lmna KO myonuclei show a progressive increase in the amount of severe DNA damage during myofiber differentiation. n=3 independent cell lines per genotype. FIG. 14C shows the quantification of DNA-PK activity in Lmna WT and Lmna KO myotubes at day 5 of differentiation by probing for the phosphorylation of DNA-PK at S2053, an autophosphorylation specific site. n=3 lysates from independent cell lines. **, p<0.01 vs. Lmna WT. FIG. 14D shows representative images of γH2AX foci in isolated single muscle fibers from Lmna WT and Lmna KO mice. Scale bar: 10 μm. FIG. 14E shows the quantification of the extent of DNA damage based on the number of γH2AX foci per nucleus in isolated single fibers. n=3-5 mice per genotype in which 5 fibers are imaged per animal. FIG. 14F shows representative image of p-DNA-PK (S2053) in isolated muscle fibers from Lmna WT and Lmna KO mice. Scale bar: 20 μm. FIG. 14G shows representative image of γH2AX foci following treatment with phleomycin, with or without DNA-PK+ATM inhibition. Scale bar: 10 μm. FIG. 14H shows the quantification of the extent of DNA damage based on the number of γH2AX foci per nucleus for Lmna WT cells treated with phleomycin, with or without DNA-PK+ATM inhibition. Inhibiting DNA damage repair in the presence of phelomycin results in a significant increase in DNA damage above that of phelomycin alone. n=3 independent experiments per condition. FIG. 14I shows the quantification of cellular viability in Lmna WT myofibers using MTT assay following DNA damage induction with phleomycin, with and without concurrent treatment with DNA-PKi (NU7441) and/or ATMi (KU55933). n=3 independent experiments per condition. **, p<0.01 vs. untreated control; *, p<0.05 vs. untreated control. Dashed red line indicates the corresponding quantity of the Lmna KO untreated control.

FIG. 15 demonstrates that Lmna KO myotendinous junction myonuclei have increased DNA-PK activity in vivo. Quantification of p-DNA-PKcs immunofluorescence in isolated muscle fibers from Lmna and Lmna KO mice. n=4-5 per genotype.

FIG. 16 demonstrates that the Lmna KO myonuclei with the highest amount of γH2AX foci frequently display chromatin protrusions. Analysis of DNA damage, assessed by γH2AX staining, in Lmna KO nuclei, comparing nuclei with chromatin protrusions to those without protrusions. Chromatin protrusions were assessed based on the presence of chromatin extending beyond the nuclear envelope, marked by lamin B-staining, n=3. **,p<0.01 vs. no protrusion.

FIGS. 17A-17B demonstrate that Lmna KO myotubes have no defects in DNA damage repair. FIG. 17A shows representative images of γH2AX foci in Lmna WT and Lmna KO myotubes at 3, 6 and 24 hours following a 5 Gy dose with radiation or no irradiation control.

FIG. 17B shows the quantification of γH2AX after 3, 6 and 24 hours post-irradiation or no irradiation control. n=3 independent cell lines. ***, p<0.001 vs. control.

FIG. 18 demonstrates that inducing DNA damage or inhibiting DNA repair does not promote additional cells death in Lmna KO myofibers. Quantification of cellular viability in Lmna KO myofibers using MTT assay following DNA damage induction with phleomycin, with and without concurrent treatment with DNA-PKi (NU7441) and/or ATMi (KU55933). n=3 independent experiments per condition.

FIG. 19 demonstrates that microtubules form cage-like structures around myonuclei. Representative immunofluorescence image of an isolated Lmna WT muscle fiber stained for tubulin (magenta), F-actin (green), and DNA (blue), showing characteristic ‘microtubule cage’ around myonucleus. Scale bar: 5 μm.

FIGS. 20A-20D demonstrate that mechanical reinforcement of Lmna KO myonuclei by microtubule stabilization reduces nuclear damage. FIG. 20A is a representative image of nuclear deformation following microharpoon in Lmna KO myotubes at day five of differentiation. Myotubes were treated for 24 hours with either paclitaxel or DMSO control. Yellow dotted line denotes the perimeter of the nucleus prior to strain application. Scale bar: 20 μm. FIG. 20B shows the quantification of nuclear strain in Lmna WT and Lmna KO myofibers using microharpoon assay following 24 hours of treatment with 50 nM paclitaxel or DMSO vehicle control. *, p<0.05 vs. Lmna WT. \, p<0.05 vs. vehicle control. FIG. 20C shows the quantification of chromatin protrusions at day 7 of differentiation following treatment with the paclitaxel (50 nM) or DMSO starting at day 4. n=3 independent experiments. ***, p<0.001 vs. Lmna WT. \, p<0.01 vs. vehicle control. FIG. 20D shows the quantification of cGAS-mCherry foci formation during 10 myofiber differentiation following treatment with paclitaxel (10 nM) or DMSO control, starting at day 5 of differentiation. n=3 independent experiments.

FIGS. 21A-21B demonstrate that inhibiting myofiber contractility does not prevent nuclear envelope rupture in Lmna KO myofibers. FIG. 21 A shows the quantification of cGAS-mCherry foci formation during 10 day myofiber differentiation follow treatment with nifedipine (5 μM) which inhibits contractility, or DMSO vehicle control, starting at day 5 of differentiation. N=3 independent experiments. FIG. 21B shows the quantification of chromatin protrusions at day 7 of differentiation following treatment with nifedipine (5 μM) or DMSO, starting at day 4. Data generated from n=3 independent experiments in which 27-53 nuclei were analyzed per genotype.

FIG. 22 demonstrates that the fraction of nuclei with severe chromatin protrusions increased over time in Lmna mutant myofibers. FIG. 22 shows the quantification of the relative distribution of chromatin protrusion lengths in Lmna KO, Lmna N195K and Lmna H222P muscle cells at day 5 and day 10 of in vitro differentiation.

FIGS. 23A-23H demonstrate that reducing cytoskeletal forces on myonuclei prevents nuclear envelope rupture and improves viability and contractility in Lmna KO myotubes. FIG. 23A is a representative time-lapse image sequence of nuclear envelope rupture in Lmna KO myonuclei during nuclear migration at day five of differentiation. White and yellow arrowheads mark two individual nuclei that undergo nuclear envelope rupture, visible by transient loss of NLS-GFP from the nucleus and stable accumulation of cGAS-mCherry at the site of rupture (red arrow). Images on the right show close-ups of the nucleus marked with a yellow arrowhead. Scale bar: 10 μm for all images. FIG. 23B show representative images of cGAS-mCherry accumulation in Lmna KO cells treated with either non-target control siRNA (siRNA NT) or siRNA against Kif5b. Scale bar: 20 μm. FIG. 23C shows the quantification of the number of Lmna KO myonuclei positive for cGAS-mCherry foci following treatment with either non-target siRNA (siRNA NT) or siRNA against Kif5b. n=3 independent experiments, in which a total of 911-1383 nuclei per condition were quantified. **, p<0.01 vs. NT. FIG. 23D shows the quantification of the number of cGAS-mCherry foci in Lmna KO expressing either DN-KASH or DN-KASHext, treated with or without 1 μM doxycycline (DOX) starting at day 3 until day 10 of differentiation. n=3 independent experiments per condition. FIG. 23E shows the quantification of the number of cGAS-mCherry foci at 10 days of differentiation as shown in FIG. 23D. **, p<0.01 vs. DN-KASH−DOX. n=3 independent experiments per condition.

FIG. 23F shows representative images of Lmna KO expressing either DN-KASH or DN-KASHext, with or without 1 μM doxycycline (DOX), and immunofluorescently labeled for Myosin Heavy Chain, Actin and DAPI showing increased cell area and enhanced sarcomeric staining in the cells expressing DN-KASH+DOX. Scale bar: 50 μm. FIG. 23G shows the quantification of cell viability following DN-KASH or DN-KASH-ext treatment in Lmna KO cells using the MTT assay. n=6 per condition from 3 independent experiments. **, p<0.01 vs. DN-KASH−DOX; p<0.001 vs DN-KASH−DOX and DN-KASH+DOX. FIG. 23H shows the quantification of myofiber contraction following DN-KASH or DN-KASHext treatment in Lmna KO cells using based on the percent of contractile fibers. n=4 independent experiments per condition. ***, p<0.001 vs. DN-KASH−DOX; tit p<0.001 vs DN-KASH−DOX and DN-KASH+DOX.

FIGS. 24A-24D demonstrate that Kif5b depletion in myotubes reduced chromatin protrusions and DNA damage in Lmna KO myonuclei. FIG. 24A is a Western blot for Kif5b in myoblasts treated with a non-target control siRNA (siRNA NT) or siRNA against Kif5b. n=3 independent experiments. The bottom panel shows the corresponding quantification. ***, p<0.001 vs. respective genotype siRNA NT control. FIG. 24B shows representative images of Lmna KO myofiber at day 5 of differentiation treated with either a non-target control siRNA (siRNA NT) or siRNA against kinesin-1 (siRNA Kif5b) at day 0. Scale bar: 20 μm. Quantification of the number of chromatin protrusions at day 5 of differentiation in Lmna KO cells treated with non-target (NT) siRNA or depleted for Kif5b using two independent siRNAs (Kif5b#3 and Kif5b#4) is shown. n=4 independent experiments, with 155-270 nuclei counted per image. ***, p<0.001 vs. NT control. FIG. 24C shows representative images of Lmna KO cells treated with either non-target (NT) siRNA or siRNA against Kif5b and immunofluorescently labeled for γH2AX, showing fewer chromatin protrusions and less DNA damage in the Kif5b depleted cells. Scale bar: 20 μm. FIG. 24D shows the quantification of the number of γH2AX foci in Lmna KO myonuclei following treatment with either non-target siRNA or siRNA against Kif5b. n=3 independent experiments in which 27-53 nuclei are counted per image.

FIGS. 25A-25F demonstrates that expression of the DN-KASH2 construct disrupts the LINC complex and limits nuclear movement, without affecting myofiber function in

Lmna WT myofibers. FIG. 25A is a representative image showing displacement of endogenous nesprin-1 in myofibers expressing the DN-KASH2 construct, and no displacement of nesprin-1 in myofibers expressing the DN-KASH2ext construct. Scale bar: 10 μm. FIG. 25B is a representative image showing nuclear clustering in myofibers expressing the DN-KASH2 construct, and normal nuclear spreading in myofibers expressing the DN-KASH2ext construct. Scale bar: 20 μm. FIG. 25C shows the quantification of cell viability following DN-KASH2 or DN-KASH2ext treatment in Lmna WT cells using the MTT assay. n=6 per condition from 3 independent experiments. FIG. 25D shows the quantification of myofiber contraction following DN-KASH2 or DN-KASH2ext treatment in Lmna WT cells based on the percent of contractile fibers. n=4 independent experiments per condition. FIG. 25E shows the quantification of the number of chromatin protrusions in Lmna KO myonuclei expressing either DN-KASH2 or DN-KASH2ext. n=3 per condition. ***, p<0.001 vs. DN-KASH2+DOX; \\\, p<0.001 vs DN-KASH2ext−DOX and DN-KASH2ext+DOX. **, p<0.01 vs. DN-KASH2ext+DOX. FIG. 25F shows the quantification of the extent of DNA damage based on the number of γH2AX foci per nucleus during myofiber differentiation. Lmna KO myonuclei expressing the DN-KASH2 construct show a decrease in the nuclei with >25 foci. n=4 per condition.

FIGS. 26A-26E demonstrate that human muscle biopsy tissue from individuals with LMNA muscular dystrophy show increased 53BP1 staining. Cryopreserved human muscle biopsy tissue from individuals with LMNA muscular dystrophy and age-matched controls was sectioned and stained with either anti-53BP1, DAPI, and phalloidin (FIG. 26A) or anti-53BP1, DAPI, and dystrophin (FIG. 26B). Yellow arrowheads denote nuclei within muscle fibers, identified by anti-dystrophin immunolabeling of the muscle fiber membrane. Each muscular dystrophy patient possesses a LMNA mutation that results in a single amino acid substitution (Table 1). The LMNA mutations cause reduced fiber size, abnormally shaped fibers, and increased nuclear 53BP1 staining. Scale bar: 30 μm. In FIGS. 26C-26E, patients were stratified based on the age at to time of muscle biopsy. The nuclear intensity values of 53BP1 were binned into 11 categories based on the level of intensity (color coding on the right). The X-axis represents the mutant lamin A/C expressed in individuals with an LMNA mutation and the age-matched control samples expressing wild-type lamin A/C. The Y-axis represents the relative percent intensity of 53BP1 staining quantified using ImageJ. Approximately 200-300 nuclei of each genotype were used for the quantification.

FIGS. 27A-27B demonstrate that human laminopathy muscle tissue shows increased 53BP1 staining. Representative images of cryopreserved human muscle biopsy tissue from individuals with LMNA muscular dystrophy and age-matched controls stained with either anti-53BP1, DAPI, and Phalloidin (FIG. 27A) or anti-53BP1, anti-dystrophin, and DAPI (FIG. 27B). The boxed regions in the left column of FIG. 27B are magnified in the right column, showing increased anti-53BP1 staining in muscle from laminopathy individuals versus age-matched controls.

FIGS. 28A-28B demonstrate the proposed mechanism by which Lmna mutations result in myofiber dysfunction and death. FIG. 28A shows that kinesin-1 motor proteins spread myonuclei along the myotubes axis during differentiation. In Lmna mutant cells, which have mechanically weaker nuclei, the localized forces associated with nuclear migration cause chromatin protrusion and nuclear envelope ruptures. This mechanically induced nuclear damage results in DNA damage, detected by H2AX foci, and activation of the DNA damage response pathways, which leads to decline in myofiber health and cell death. FIG. 28B is a schematic flow chart delineating the steps described in FIG. 28A, along with interventions explored in this work. Stabilizing microtubules surrounding the myonuclei reinforces the Lmna mutant nuclei and prevents chromatin protrusions and nuclear envelope ruptures. Inhibiting nuclear movement by Kif5b depletions similarly prevents nuclear damage. Muscle contractions may also contribute to nuclear damage in vivo.

### DETAILED DESCRIPTION

A first aspect of the present application relates to a method of treating a laminopathy affecting skeletal or cardiac muscle in a subject. This method involves selecting a subject who has a laminopathy affecting skeletal or cardiac muscle and administering, to the selected subject, an inhibitor of a protein associated with a DNA damage response (DDR) pathway to treat the laminopathy affecting skeletal or cardiac muscle in the subject.

As used herein, a “subject” or a “patient” encompasses any animal, preferably a mammal. Suitable subjects include, without limitation, domesticated and undomesticated animals such as rodents (mouse or rat), cats, dogs, rabbits, horses, sheep, pigs, and monkeys. In one embodiment the subject is a human subject. Suitable human subjects include, without limitation, an infant, a neonate, a child, an adult, and an elderly subject.

As used herein, the terms “laminopathy” or “envelopathy” refer to a disease or condition associated with a mutation in one or more genes selected from the group consisting of a Lamin A/C (LMNA), emerin (EMD), nesprin-1 (SYNE1), nesprin-2 (SYNE2), SUN domain-containing protein 1 (SUN1), and SUN domain-containing protein 2 (SUN2).

The LMNA gene encodes lamins A/C which, together with the B-type lamins, are the major components of the nuclear lamina, which line the inner nuclear membrane. Through interactions with emerin (encoded by EMD), nesprin-1 (encoded by SYNE1), nesprin-2 (encoded by SYNE2), SUN domain-containing protein 1 (encoded by SUN1), and SUN domain-containing protein 2 (encoded by SUN2) proteins, lamin A functions as a structural integrator responsible for the maintenance of nuclear shape and stability, as well as for resistance to mechanical stress, which is extremely important for tissues exposed to it, e.g. skeletal and cardiac muscles.

Attributed to nearly half of the greater than 600 described pathogenic LMNA mutations, striated muscle laminopathies are the most prevalent type of laminopathy (Miroshnikova et al., “Cell Biology and Mechanopathology of Laminopathic Cardiomyopathies,” J. Cell Biol. (2019), which is hereby incorporated by reference in its entirety). Life-limiting cardiac dysfunction is the unifying feature of these diseases that are caused by altered biophysical properties of muscle cells, resulting in thinning of ventricular walls, enlargement of ventricular volume, and impaired electrophysiological conduction. These defects compromise cardiac output and trigger malignant arrhythmias.

In some embodiments, the selected subject has a laminopathy associated with a mutation in one or more genes selected from the group consisting of Lamin A/C (LMNA), emerin (EMD), nesprin-1 (SYNE1), nesprin-2 (SYNE2), SUN domain-containing protein 1 (SUN1), and SUN domain-containing protein 2 (SUN2).

Mutations may include a deletion, an insertion, a point mutation, a missense mutation, a frame shift mutation, a truncation, a nonsense mutation, or a splice-site mutation. In some embodiments of the methods described herein, the mutation comprises a non-synonymous single nucleotide base substitution, insertion, or deletion. As used herein, the term “nonsense mutation” refers to a mutation that leads to the appearance of a stop codon in the nucleotide sequence where previously there was a codon specifying an amino acid, which results in the translation of a shortened protein. As used herein, the term “deletion” refers to a mutation that involves the loss of genetic material and results in the deletion of one or more amino acids in a protein.

In some embodiments, the laminopahty is associated with a mutation in the Lamin A/C (LMNA) gene (NCBI GeneID No: 4000). The human lamin A/C gene (LMNA) encodes prelamin A, which is processed to lamin A, lamin C, lamin A-delta10, and lamin D (see, e.g., Worman et al., “Cell Signaling Abnormalities in Cardiomyopathy Caused by Lamin A/C Gene Mutations,” Biochem. Soc. 46:37-42 (2018), which is hereby incorporated by reference in its entirety). Exemplary amino acid sequences for human lamin A, lamin A-delta10, lamin C, and laimin D are shown in Table 2 below.

TABLE 2
Exemplary Human Lamin Amino Acid Sequences

| | | SEQ | |
|---|---|---|---|
| | | ID | NCBI Ref. |
| Protein | Amino Acid Sequence | NO: | Seq. No. |
| Lamin | METPSQRRAT RSGAQASSTP | 1 | NP_733821.1 |
| A | LSPTRITRLQ EKEDLQELND | | |
| | RLAVYIDRVR SLETENAGLR | | |
| | LRITESEEVV SREVSGIKAA | | |
| | YEAELGDARK TLDSVAKERA | | |
| | RLQLELSKVR EEFKELKARN | | |
| | TKKEGDLIAA QARLKDLEAL | | |
| | LNSKEAALST ALSEKRTLEG | | |
| | ELHDLRGQVA KLEAALGEAK | | |
| | KQLQDEMLRR VDAENRLQTM | | |
| | KEELDFQKNI YSEELRETKR | | |
| | RHETRLVEID NGKQREFESR | | |
| | LADALQELRA QHEDQVEQYK | | |
| | KELEKTYSAK LDNARQSAER | | |
| | NSNLVGAAHE ELQQSRIRID | | |
| | SLSAQLSQLQ KQLAAKEAKL | | |
| | RDLEDSLARE RDTSRRLLAE | | |
| | KEREMAEMRA RMQQQLDEYQ | | |
| | ELLDIKLALD MEIHAYRKLL | | |
| | EGEEERLRLS PSPTSQRSRG | | |
| | RASSHSSQTQ GGGSVTKKRK | | |
| | LESTESRSSF SQHARTSGRV | | |
| | AVEEVDEEGK FVRLRNKSNE | | |
| | DQSMGNWQIK RQNGDDPLLT | | |
| | YRFPPKFTLK AGQVVTIWAA | | |
| | GAGATHSPPT DLVWKAQNTW | | |
| | GCGNSLRTAL INSTGEEVAM | | |
| | RKLVRSVTVV EDDEDEDGDD | | |
| | LLHHHHGSHC SSSGDPAEYN | | |
| | LRSRTVLCGT CGQPADKASA | | |
| | SGSGAQVGGP ISSGSSASSV | | |
| | TVTRSYRSVG GSGGGSFGDN | | |
| | LVTRSYLLGN SSPRTQSPQN | | |
| | CSIM | | |
| Lamin | METPSQRRAT RSGAQASSTP | 2 | NP_733822.1 |
| A- | LSPTRITRLQ EKEDLQELND | | |
| delta10 | RLAVYIDRVR SLETENAGLR | | |
| | LRITESEEVV SREVSGIKAA | | |
| | YEAELGDARK TLDSVAKERA | | |
| | RLQLELSKVR EEFKELKARN | | |
| | TKKEGDLIAA QARLKDLEAL | | |
| | LNSKEAALST ALSEKRTLEG | | |
| | ELHDLRGQVA KLEAALGEAK | | |
| | KQLQDEMLRR VDAENRLQTM | | |
| | KEELDFQKNI YSEELRETKR | | |
| | RHETRLVEID NGKQREFESR | | |
| | LADALQELRA QHEDQVEQYK | | |
| | KELEKTYSAK LDNARQSAER | | |
| | NSNLVGAAHE ELQQSRIRID | | |
| | SLSAQLSQLQ KQLAAKEAKL | | |
| | RDLEDSLARE RDTSRRLLAE | | |
| | KEREMAEMRA RMQQQLDEYQ | | |
| | ELLDIKLALD MEIHAYRKLL | | |
| | EGEEERLRLS PSPTSQRSRG | | |
| | RASSHSSQTQ GGGSVTKKRK | | |
| | LESTESRSSF SQHARTSGRV | | |
| | AVEEVDEEGK FVRLRNKSNE | | |
| | DQSMGNWQIK RQNGDDPLLT | | |
| | YRFPPKFTLK AGQVVTIWAA | | |
| | GAGATHSPPT DLVWKAQNTW | | |
| | GCGNSLRTAL INSTGEGSHC | | |
| | SSSGDPAEYN LRSRTVLCGT | | |
| | CGQPADKASA SGSGAQVGGP | | |
| | ISSGSSASSV TVTRSYRSVG | | |
| | GSGGGSFGDN LVTRSYLLGN | | |
| | SSPRTQSPQN CSIM | | |
| Lamin | METPSQRRAT RSGAQASSTP | 3 | NP_005563.1 |
| C | LSPTRITRLQ EKEDLQELND | | |
| | RLAVYIDRVR SLETENAGLR | | |
| | LRITESEEVV SREVSGIKAA | | |
| | YEAELGDARK TLDSVAKERA | | |
| | RLQLELSKVR EEFKELKARN | | |
| | TKKEGDLIAA QARLKDLEAL | | |
| | LNSKEAALST ALSEKRTLEG | | |
| | ELHDLRGQVA KLEAALGEAK | | |
| | KQLQDEMLRR VDAENRLQTM | | |
| | KEELDFQKNI YSEELRETKR | | |
| | RHETRLVEID NGKQREFESR | | |
| | LADALQELRA QHEDQVEQYK | | |
| | KELEKTYSAK LDNARQSAER | | |
| | NSNLVGAAHE ELQQSRIRID | | |
| | SLSAQLSQLQ KQLAAKEAKL | | |
| | RDLEDSLARE RDTSRRLLAE | | |
| | KEREMAEMRA RMQQQLDEYQ | | |
| | ELLDIKLALD MEIHAYRKLL | | |
| | EGEEERLRLS PSPTSQRSRG | | |
| | RASSHSSQTQ GGGSVTKKRK | | |
| | LESTESRSSF SQHARTSGRV | | |
| | AVEEVDEEGK FVRLRNKSNE | | |
| | DQSMGNWQIK RQNGDDPLLT | | |
| | YRFPPKFTLK AGQVVTIWAA | | |
| | GAGATHSPPT DLVWKAQNTW | | |
| | GCGNSLRTAL INSTGEEVAM | | |
| | RKLVRSVTVV EDDEDEDGDD | | |
| | LLHHHHVSGS RR | | |
| Lamin | MGNSEGCNTK KEGDLIAAQA | 4 | NP_00124430 |
| D | RLKDLEALLN SKEAALSTAL | | 3.1 |
| | SEKRTLEGEL HDLRGQVAKL | | |
| | EAALGEAKKQ LQDEMLRRVD | | |
| | AENRLQTMKE ELDFQKNIYS | | |
| | EELRETKRRH ETRLVEIDNG | | |
| | KQREFESRLA DALQELRAQH | | |
| | EDQVEQYKKE LEKTYSAKLD | | |
| | NARQSAERNS NLVGAAHEEL | | |
| | QQSRIRIDSL SAQLSQLQKQ | | |
| | LAAKEAKLRD LEDSLARERD | | |
| | TSRRLLAEKE REMAEMRARM | | |
| | QQQLDEYQEL LDIKLALDME | | |
| | IHAYRKLLEG EEERLRLSPS | | |
| | PTSQRSRGRA SSHSSQTQGG | | |
| | GSVTKKRKLE STESRSSFSQ | | |
| | HARTSGRVAV EEVDEEGKFV | | |
| | RLRNKSNEDQ SMGNWQIKRQ | | |
| | NGDDPLLTYR FPPKFTLKAG | | |
| | QVVTIWAAGA GATHSPPTDL | | |
| | VWKAQNTWGC GNSLRTALIN | | |
| | STGEEVAMRK LVRSVTVVED | | |
| | DEDEDGDDLL HHHHGSHCSS | | |
| | SGDPAEYNLR SRTVLCGTCG | | |
| | QPADKASASG SGAQVGGPIS | | |
| | SGSSASSVTV TRSYRSVGGS | | |
| | GGGSFGDNLV TRSYLLGNSS | | |
| | PRTQSPQNCS IIQEMGMRWE | | |
| | VEEGRRKVSL SCLP | | |

As described herein, the laminopathy may be associated with a mutation in the LMNA gene (NCBI GeneID No: 4000) corresponding to a N195K or H222P substitution in SEQ ID NO: 1 (see, e.g., Worman et al., “Cell Signaling Abnormalities in Cardiomyopathy Caused by Lamin A/C Gene Mutations,” Biochem. Soc. 46:37-42 (2018), which is hereby incorporated by reference in its entirety).

In some embodiments the laminopathy is a striated muscle laminopathy associated with a mutation in the LNMA gene (NCBI GeneID No: 4000) corresponding to an amino acid substitution in SEQ ID NO: 1 selected from the group consisting of Y45C, R5OH, G125A, R190, F206L, R249W, K270K, R331Q, R335Q, Q353R, Q355, R377C, R386K, G413C, R427G, G449V, N456H, N4561, N459S, R471H, L489P, W514R, T528K, T528R, R541H, D559Y, V586M, R644C, and combinations thereof. The present application also encompasses mutations in genes selected from the group consisting of EMD (NCBI GeneID No: 2010), SYNE1 (NCBI GeneID No: 23345), SYNE2 (NCBI GeneID No: 23224), SUN1 (NCBI GeneID No: 23353), and SUN2 (NCBI GeneID No: 25777).

In some embodiments, the laminopathy is a striated muscle laminopathy muscle laminopathy selected from the group consisting of Emery-Dreifuss muscular dystrophy (EDMD), LMNA-related congenital muscular dystrophy (LMNA-CMD), limb-girdle muscular dystrophy type 1B (LGMD1B), dilated cardiomyopathy (DCM), and dilated cardiomyopathy with conduction system defects.

Emery-Dreifuss muscular dystrophy (EDMD) primarily affects muscles used for movement (skeletal muscles) and the heart (cardiac muscle). Among the earliest features of this disorder are joint deformities called contractures. Contractures restrict the movement of certain joints, most often the elbows, ankles, and neck, and usually become noticeable in early childhood. Most affected individuals also experience muscle weakness and wasting that worsen slowly over time, beginning in muscles of the upper arms and lower legs and later also affecting muscles in the shoulders and hips. Almost all people with Emery-Dreifuss muscular dystrophy develop heart problems by adulthood. In many cases, these heart problems are abnormalities of the electrical signals that control the heartbeat (cardiac conduction defects) and abnormal heart rhythms (arrhythmias). If untreated, these abnormalities can lead to a sensation of fluttering or pounding in the chest (palpitations), an unusually slow heartbeat (bradycardia), fainting (syncope), heart failure, and an increased risk of sudden death. Thus, in some embodiments, the laminopathy is Emery-Dreifuss muscular dystrophy (EDMD).

When the laminopathy is EDMD, the subject may have a mutation in the LNMA gene (NCBI GeneID No: 4000) corresponding to an amino acid substitution in SEQ ID NO: 1 selected from the group consisting of G232E, L248P, R249Q, R249W, F260L, Y267C, S268P, L271P, Q294P, S295P, S303P, R336Q, R343Q, E358K, E361K, M371K, R377L, R386K, R401C, V442A, G449D, R453W, L454P, N456I, N456K, D461Y, W467R, I469T, W520S, R527P, T528K, T528R, L529P, L530P, R541H, R541S, R541P, G602S, R624H, and combinations thereof (Kang et al., “Laminopathies; Mutations on Single Gene and Various Human Genetic Diseases,” BMB Rep. 51(7):327-337 (2018), which is hereby incorporated by reference in its entirety). In certain embodiments, the subject has a mutation in the LNMA gene (NCBI GeneID No: 4000) corresponding to an amino acid mutation in SEQ ID NO: 1 selected from the group consisting of R25P, R25G,K32x, E33G, E33D, L35V, N39S, N39D, R41S, A43T, Y45C, I46V, D47H, R50S, R50P, I63S, I63N, E65G, L85P, R89L, R89C, L102Q, A130P, R133P, L140P, T150P, L162P, N195D, H222Y, H222P, R225Q, G232E, G232R, L248P, R249W, R249Q, Y259D, K260E, K261x, L263P, Y267H, Y267C, S268P, K270K, L271P, S277P, Q294P, S295P, S303P, S326T, R336Q, M348I, Q355X, L356R, E358K, E361K, M371K, R377L, R377H, R377C, E381A, G382R, R386M, R386K, R401C, D446V, G449D, R453W, L454P, N456K, N456I, N456H, D461Y, W467R, I469T, W498R, L512P, Q517X, W520S, W520G, R527P, T528K, T528R, L530P, R541H, R545C, D596N, G602S, R624H, R644C, and combinations thereof, wherein X indicates a nonsense mutation and wherein x indicates a deletion.

Congenital muscular dystrophy refers to a group of inhereited diseases that cause progressive weakeness and loss of muscle mass. In some embodiments, the laminopathy is LMNA-related congenital muscular dystrophy (LMNA-CMD). In subjects with LMNA-CMD, muscle weakness becomes apparent in infancy or early childhood and can worsen quickly. The most severely affected infants develop few motor skills, and they are never able to hold up their heads, roll over, or sit. Less severely affected children may learn to sit, stand, and walk before muscle weakness becomes apparent. As other skeletal muscles become weaker, these children may ultimately lose the ability to sit, stand, and walk unassisted. Other features of LMNA-CMD often include spinal rigidity and abnormal curvature of the spine (scoliosis and lordosis); joint deformities (contractures) that restrict movement, particularly in the hips and legs; and an inward-turning foot. Subjects with LMNA -CMD also have an increased risk of heart rhythm abnormalities (arrhythmias). Over time, muscle weakness causes most infants and children with LMNA -CMD to have trouble eating and breathing. The breathing problems result from restrictive respiratory insufficiency, which occurs when muscles in the chest are weakened and the ribcage becomes increasingly rigid. This problem can be life-threatening, and many affected children require support with a machine to help them breathe (mechanical ventilation).

When the laminopathy is LMNA-related congenital muscular dystrophy (LMNA-CMD), the subject may have a mutation in the LNMA gene (NCBI GeneID No: 4000) corresponding to an amino acid substitution in SEQ ID NO: 1 selected from the group consisting of N39S, R50P, R249W, L302P, E358K, L380S, R453P, R455P, N456D, and combinations thereof (Kang et al., “Laminopathies; Mutations on Single Gene and Various Human Genetic Diseases,” BMB Rep. 51(7):327-337 (2018), which is hereby incorporated by reference in its entirety). In certain embodiments, the subject has a mutation in SEQ ID NO: 1 selected from the group consisting of R28Q, K32E, K32x, L35P, N39Y, N39S, R41C, R50P, R249W, R249Q, L292P, L302P, E358K, L380S, R388C, R453P, R455P, N456D, T528R, R644C, R644H, and combinations thereof, wherein x indicates a deletion.

Limb-girdle muscular dystrophy type 1B (LGMD1B) is associated with muscle weakness in the lower limbs. The muscle weakness typically affects the muscles closest to the center of the body (proximal muscles) such as the upper legs. The disease is progressive, leading to a loss of muscle strength and bulk over a number of years. Limb-girdle muscular dystrophy type 1B is caused by mutations (changes) to the LMNA gene and is inherited in an autosomal dominant manner. Thus, in some embodiments, the laminopathy is Limb-girdle muscular dystrophy type 1B (LGMD1B).

When the laminopathy is LGMD1B, the subject may have a mutation in the LNMA gene (NCBI GeneID No: 4000) corresponding to an amino acid substitution in SEQ ID NO: 1 selected from the group consisting of R25G, Y259X, E358K, R377H, R377L, R399C, Y481H, and combinations thereof (Kang et al., “Laminopathies; Mutations on Single Gene and Various Human Genetic Diseases,” BMB Rep. 51(7):327-337 (2018), which is hereby incorporated by reference in its entirety). In certain embodiments, the subject has a mutation in the LLAMA gene (NCBI GeneID No: 4000) corresponding to an amino acid mutation in SEQ ID NO: 1 selected from the group consisting of R25G, T271, R28Q, E33G, R50S, E65G, R101P, K171K,K208x, R249Q, Y259X, A278T, L292P, S303P, K311R, Q312H, R331P, R377C, R377H, R377L, L379F, R453W, Y481H, Q493X, W498C, L512P, W514R, R527P, T528K, R541S, R541P, D596N, D639G, R644C, and combinations thereof, wherein X indicates a nonsense mutation and wherein x indicates a deletion.

Dilated cardiomyopathy (DCM) is a disease of the heart muscle which primarily affects the heart's main pumping chamber, the left ventricle. It is the most common type of cardiomyopathy and typically affects those aged 20 to 60. The left ventricle of affected individuals becomes enlarged (dilated) and cannot pump blood to the body with as much force as a healthy heart can. The heart muscle also has difficulty contracting normally, which can lead to irregular heartbeats (arrhythmia), blood clots, or sudden death. Over time, the heart becomes weaker and heart failure can occur. In some embodiments, the laminopathy is dilated cardiomyopathy (DCM).

When the laminopathy is dilated cardiomyopathy (DCM), the subject may have a mutation in the LLAMA gene (NCBI GeneID No: 4000) corresponding to an amino acid substitution in SEQ ID NO: 1 selected from the group consisting of Q6X, R25G, R25P, R25W, R25G, E33G, L35V, N39S, A43T, Y45C, R50S, L59R, R60G, I63N, I63S, E65G, E82K, L85R, R89L, R89C, K97E, R133P, S143P, E161K, L140P, T150P, R189P, R190Q, R190W, D192G, N195K, R196S, E203K, E203G, L215P, H222P, H222Y, R225X, Y267C, E317K, A347K, R349L, Q355X, R399C, R435C, R541C, R541S, S573L, R644C, and combinations thereof (Kang et al., “Laminopathies; Mutations on Single Gene and Various Human Genetic Diseases,” BMB Rep. 51(7):327-337 (2018), which is hereby incorporated by reference in its entirety).

Dilated cardiomyopathy with conduction system defects (DCM-CD) involves degeneration of the hearts conduction system. Conduction system involvement usually starts with disease of the sinus node and/or atrioventricular node that can manifest as sinus bradycardia, sinus node arrest with junctional rhythms, or heart block (commonly first-degree heart block that progresses to second- and third-degree block) (Hershberger et al., “LMNA-Related Dilated Cardiomyopathy,” in GeneReviews® [Internet] Seattle (WA): University of Washington, Seattle; 1993-2019, which is hereby incorporated by reference in its entirety). Symptomatic bradyarrhythmias requiring cardiac pacemakers; supraventricular arrhythmias including atrial flutter, atrial fibrillation, supraventricular tachycardia, and the sick sinus syndrome (i.e., tachycardia-bradycardia syndrome); and ventricular arrhythmias including frequent premature ventricular contractions (PVCs), ventricular tachycardia, and ventricular fibrillation are also common. In some embodiments, the laminopathy is dilated cardiomyopathy with conduction system defects.

When the laminopathy is dilated cardiomyopathy with conduction system defects (DCM-CD), the subject may have a mutation in the LNMA gene (NCBI GeneID No: 4000) corresponding to an amino acid mutation in SEQ ID NO: 1 selected from the group consisting of Q6X, S22L, R28W, Q36X, Y45C, L52P, E53V, R60G, E82K, L85R, R89L, T91T, L92F, K97E, R101P, R110S, E11X, K117R, K123x, A132P, S143P, E161K, R166P, L183P, E186K, R189W, R190W, R190Q, D192G, D192V, N195K, N195K, E203K, E203G, E203V, I210S, L215P, K219T, K219N, R225X, Q234X, Q246X, Y259H, K260N, Y267H, A278T, E291K, Q312H, E317K, A318T, R321X, R331Q, R335W, R335Q, E347K, M348I, R349L, A350P, Q355X, D357H, D357A, Q358X, R377H, R377L, R388H, R399C, R435C, Q432X, V440M, D461Y, R471, Y481X, Q517X, W520X, G523R, R541S, R541G, R541C, R541H, R541P, S571R, S573L, A617A, G635D, R644C, R654X, and combinations thereof, wherein X indicates a nonsense mutation and wherein x indicates a deletion.

Mutations in the LMNA gene are also associated with various progeroid diseases. For example, Hutchinson-Gilford progeria syndrome (HGPS) is caused by mutations of the LMNA gene leading to increased production of a partially processed form of the nuclear fibrillar protein lamin A-progerin. Progerin acts as a dominant factor that leads to multiple morphological anomalies of cell nuclei and disturbances in heterochromatin organization, mitosis, DNA replication and repair, and gene transcription (Ashapkin et al., “Are There Common Mechanisms Between the Huchinson-Gilford Progeria Syndrome and Natural Aging?” Front. Genet. 10:455 (2019), which is hereby incorporated by reference in its entirety). The premature aging in HGPS patients is mediated by changes in the activity of signaling pathways, including downregulation of DNA repair and chromatin organization, and upregulation of mTOR.

In some embodiments, the methods of the present application do not contemplate laminopathies comprising a mutation in the LMNA gene associated with progeriod diseases. Thus, according to some embodiments, the selected subject does not have a laminopathy associated with a progeroid disease. In certain embodiments, the mutation in the LMNA gene is not associated with a progeroid disease or the expression of progerin.

In carrying out the methods described herein, “treating” or “treatment” includes inhibiting, preventing, ameliorating, or delaying onset of a particular disease or condition (e.g., a laminopathy selected from the group consisting of Emery-Dreifuss muscular dystrophy (EDMD), congenital muscular dystrophy, limb-girdle muscular dystrophy type 1B, dilated cardiomyopathy, and dilated cardiomyopathy). Treating and treatment also encompasses any improvement in one or more symptoms of the condition or disorder. Treating and treatment encompasses any modification to the condition or course of disease progression as compared to the condition or disease in the absence of therapeutic intervention. Thus, in some embodiments, treating the subject improves muscle strength, reduces muscle wasting, and/or reduces muscle death in a subject who has a laminopathy affecting cardiace or skeletal muscle.

According to some embodiments, administering one or more of the therapeutic agents (i.e., an inhibitor of a protein associated with a DNA damage response (DDR) pathway, a microtubule stabilizing agent, and/or a LINC complex disruptor of the present application) is effective to reduce at least one symptom of a disease or condition that is associated with a laminopathy affecting skeletal or cardiac muscle in a subject. In other embodiments, the administering is effective to mediate an improvement in the disease or condition that is associated with a laminopathy affecting skeletal or cardiac muscle in a subject. In further embodiments, the administering is effective to prolong survival in the subject as compared to expected survival if no administering were carried out.

As described herein, the “DNA damge response (DDR) pathway” refers to a network of signaling cascades leading to the activation of cell cycle checkpoints, DNA repair, and apoptosis. The DNA damage response is coordinated by phosphatidylinositol 3-kinase-related kinases (PIKKs), which include ataxia-telangiectasia mutated (ATM), ATM- and Rad3-Related (ATR), DNA-dependent protein kinase (DNA-PK), and suppressor with morphogenetic effect on genitalia (SMG-1) (see, e.g., Blackford et al., “ATM, ATR, and DNA-PK: The Trinity at the Heart of the DNA Damage Response,” Mol. Cell 66(6):801-817 (2017), which is hereby incorporated by reference in its entirety). ATM is primarily activated by double-stranded DNA breaks (DSBs); ATR responds to a broad spectrum of DNA damage, including DSBs and a variety of DNA lesions that interfere with replication; DNA-PK regulates a smaller number of targets and play a role primarily in nonhomologous end joining (NHEJ), and SMG-1 is activated by DSBs (Marechal et al., “DNA Damage Sensing by the ATM and ATR Kinases,” Cold Spring Harb. Perspect. Biol. 5(9):a012716 and Gewandter et al., “The RNA Surveillance Protein SMG1 Activates p53 in Response to DNA Double-Strand Breaks but not Exogenously Oxidized mRNA,” Cell Cycle 10(15):2561-2567, which are hereby incorporated by reference in their entirety).

As described herein, Applicants have surprisingly found that inhibitors of the DNA damge response (DDR) pathway inhibit the progressive nuclear envelope damage associated with laminopathies affecting skeletal and/or cardiac muscle. Thus, in some embodiments, the selected subject is administered an inhibitor of a protein associated with the DDR pathway.

As used herein, an “inhibitor” refers to a compound or composition that reduces a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

An “inhibitor of a protein associated with the DDR pathway” refers to a compound or composition that inhibits the activity of a protein associated with the DDR pathway. In some embodiments, the inhibitor of a protein associated with the DDR pathway is a small molecule, a protein, a peptide, a nucleic acid, an aptamer, an antibody, or a derivative thereof.

In some embodiments, the inhibitor of a protein associated with the DDR pathway is a nucleic acid selected from the group consisting of siRNA, shRNA, miRNA.

siRNAs are double stranded synthetic RNA molecules approximately 20-25 nucleotides in length with short 2-3 nucleotide 3′ overhangs on both ends. The double stranded siRNA molecule represents the sense and anti-sense strand of a portion of a target mRNA molecule. In some some embodiments, the siRNA molecules represent a the sense and anti-sense of a portion of a mRNA molecule encoding a protein associated with the DDR pathway (e.g., ATR, ATM, DNA-PK, and/or SMG-1). The sequence of various mRNA molecules encoding a protein associated with the DDR pathway are readily known in the art and accessible to one of skill in the art for the purposes of designing siRNA oligonucleotides.

siRNA molecules are typically designed to target a region of the mRNA target approximately 50-100 nucleotides downstream from the start codon. Methods and online tools for designing suitable siRNA sequences based on the target mRNA sequences are readily available in the art (see e.g., Reynolds et al., “Rational siRNA Design for RNA Interference,” at. Biotech. 2:326-330 (2004); Chalk et al., “Improved and Automated Prediction of Effective siRNA,” Biochem. Biophys. Res. Comm. 319(1): 264-274 (2004); Zhang et al., “Weak Base Pairing in Both Seed and 3′ Regions Reduces RNAi Off-targets and Enhances si/shRNA Designs,” Nucleic Acids Res. 42(19):12169-76 (2014), which are hereby incorporated by reference in their entirety). Upon introduction into a cell, the siRNA complex triggers the endogenous RNA interference (RNAi) pathway, resulting in the cleavage and degradation of the target mRNA molecule. Various improvements of siRNA compositions, such as the incorporation of modified nucleosides or motifs into one or both strands of the siRNA molecule to enhance stability, specificity, and efficacy, have been described and are suitable for use in accordance with this aspect of the invention (see e.g., WO2004/015107 to Giese et al.; WO2003/070918 to McSwiggen et al.; WO1998/39352 to Imanishi et al.; U.S. Patent Application Publication No. 2002/0068708 to Jesper et al.; U.S. Patent Application Publication No. 2002/0147332 to Kaneko et al; U.S. Patent Application Publication No. 2008/0119427 to Bhat et al., which are hereby incorporated by reference in their entirety). Methods of constructing DNA-vectors for siRNA expression in mammalian cells are known in the art, see e.g., Sui et al., “A DNA Vector-Based RNAi Technology to Suppress Gene Expression in Mammalian Cells,” Proc. Nat'l Acad. Sci. USA 99(8):5515-5520 (2002), which is hereby incorporated by reference.

Short or small hairpin RNA (shRNA) molecules are similar to siRNA molecules in function, but comprise longer RNA sequences that make a tight hairpin turn. shRNA is cleaved by cellular machinery into siRNA and gene expression is silenced via the cellular RNA interference pathway. Methods and tools for designing suitable shRNA sequences based on the target mRNA sequences (e.g., ATR, ATM, DNA-PK, and/or SMG-1 mRNA sequences) are readily available in the art (see e.g., Taxman et al., “Criteria for Effective Design, Constructions, and Gene Knockdown shRNA Vectors,” BMC Biotech. 6:7 (2006) and Taxman et al., “Short Hairpin RNA (shRNA): Design, Delivery, and Assessment of Gene Knockdown,” Meth. Mol. Biol. 629: 139-156 (2010), which are hereby incorporated by reference in their entirety). Methods of constructing DNA-vectors for shRNA expression and gene silencing in mammalian cells is described herein and are known in the art, see e.g., Cheng and Chang, “Construction of Simple and Efficient DNA Vector-based Short Hairpin RNA Expression Systems for Specific Gene Silencing in Mammalian Cells,” Methods Mol. Biol. 408:223-41 (2007), which is hereby incorporated by reference in its entirety.

Other suitable agents that can be used for purposes of inhibiting a protein associated with a DNA damage response (DDR) pathway include microRNAs (miRNAs). miRNAs are small, regulatory, noncoding RNA molecules that control the expression of their target mRNAs predominantly by binding to the 3′ untranslated region (UTR). A single UTR may have binding sites for many miRNAs or multiple sites for a single miRNA, suggesting a complex post-transcriptional control of gene expression exerted by these regulatory RNAs (Shulka et al., “MicroRNAs: Processing, Maturation, Target Recognition and Regulatory Functions,” Mol. Cell. Pharmacol. 3(3):83-92 (2011), which is hereby incorporated by reference in its entirety). Mature miRNA are initially expressed as primary transcripts known as a pri-miRNAs which are processed, in the cell nucleus, to 70-nucleotide stem-loop structures called pre-miRNAs by the microprocessor complex. The dsRNA portion of the pre-miRNA is bound and cleaved by Dicer to produce a mature 22 bp double-stranded miRNA molecule that can be integrated into the RISC complex; thus, miRNA and siRNA share the same cellular machinery downstream of their initial processing.

Other suitable agents that can be used for purposes of inhibiting a protein associated with a DNA damage respone (DDR) pathway include antisense nucleotides. The use of antisense methods to inhibit the in vivo translation of genes and subsequent protein expression is well known in the art (e.g., U.S. Pat. No. 7,425,544 to Dobie et al.; U.S. Pat. No. 7,307,069 to Karras et al.; U.S. Pat. No. 7,288,530 to Bennett et al.; U.S. Pat. No. 7,179,796 to Cowsert et al., which are hereby incorporated by reference in their entirety). Antisense nucleic acids are nucleic acid molecules (e.g., molecules containing DNA nucleotides, RNA nucleotides, or modifications (e.g., modification that increase the stability of the molecule, such as 2′-0-alkyl (e.g., methyl) substituted nucleotides) or combinations thereof) that are complementary to, or that hybridize to, at least a portion of a specific nucleic acid molecule, such as an mRNA molecule (see e.g., Weintraub, H. M., “Antisense DNA and RNA,” Scientific Am. 262:40-46 (1990), which is hereby incorporated by reference in its entirety). The antisense nucleic acid molecule hybridizes to its corresponding target nucleic acid molecule (e.g., an mRNA molecule encoding a protein associated with the DDR pathway (e.g., ATR, ATM, DNA-PK, and/or SMG-1)), to form a double-stranded molecule, which interferes with translation of the mRNA, as the cell will not translate a double-stranded mRNA. Antisense nucleic acids used in the methods of the present invention are typically at least 10-15 nucleotides in length, for example, at least 10, 11, 12, 13, 14, 15, 16, 17, 18, 19, 20, 25, 30, 35, 40, 45, 50, 55, 60, 65, 70, 75, or greater than 75 nucleotides in length. The antisense nucleic acid can also be as long as its target nucleic acid with which it is intended to form an inhibitory duplex.

In some embodiments, the inhibitors described herein reduce the expression of one or more endogenous target proteins. The expression of the one or more endogenous target proteins may be reduced by 5%, 6%, 7%, 8%, 9%, 10%, 20%, 30%, 40%, 50%, 60%, 70%, 80%, 90%, 95%, 99%, 99.5%, 99.9%, or 100% relative to the wildtype level of expression.

According to one embodiment, the inhibitor of a protein associated with the DDR pathway is selected from the group consisting of an antibody, a Fab fragment, a F(ab)₂ fragment, a Fab′ fragment, a F(ab′)₂ fragment, a Fd fragment, a Fd′ fragment, and a Fv fragment.

The protein associated with the DNA damage response (DDR) pathway may be a phosphatidylinositol 3-kinase-related kinase (PIKK). Thus, in some embodiments, the PIKK is selected from the group consisting of DNA-dependent protein kinase (DNA-PK), ataxia telangiectasia mutated serine-protein kinase (ATM), ataxia telangiectasia and Rad3-related (ATR), suppressor of morphogenesis in genitalia-1 (SMG-1), and combinations thereof.

The mammalian target of rapamycin (mTOR) is a member of the phosphatidylinositol 3-kinase-related kinase (PIKK) family and is a major regulator of translation, cell growth, and autophagy. However, the role of mTOR in the DNA damage response pathway is unclear (Lamm et al., “The mTOR Pathway: Implications for DNA Replication,” Prog. Biophys. Mol. Biol. (2019), which is hereby incorporated by reference in its entirety). Therefore, for purposes of the present application, the mTOR is not the PIKK.

As described supra, DNA-PK is activated upon association with DNA. DNA-PK is composed of a large catalytic subunit (DNA-PKcs) and a regulatory heterdimer (Ku70/Ku80). The DNA-PKcs subunit comprises a nuclear serine/threonine protein kinase. The Ku heterodimer binds to DNA double-strand break ends and is required for the nonhomologous end joining (NHEJ) pathway of NA repair. In some embodiments, where the phosphatidylinositol 3-kinase-related kinase (PIKK) is a DNA-dependent protein kinase, the inhibitor may selectively target a DNA-PK catalytic subunit (DNA-PKcs), Ku70, and/or Ku80.

In some embodiments, when the phosphatidylinositol 3-kinase-related kinase (PIKK) is a DNA-dependent protein kinase (DNA-PK), the inhibitor is selected from the group consisting of NU7441, NU7026, LY294002, IC86621, IC87102, IC87361, OK-1035, SU1172, NK314, IC486241, vanillin, wortmannin, and GRN163L (see, e.g., Davidson et al., “Small Molecules, Inhibitors of DNA-PK, Targeting DNA Repair, and Beyond,” Front. Pharmacol. 4:4 (2013), which is hereby incorporated by reference in its entirety).

In some embodiments, when the phosphatidylinositol 3-kinase-related kinase

(PIKK) is ataxia telangiectasia mutated serine-protein kinase (ATM), the inhibitor may be an ATM inhibitor selected from the group consisting of KU55933, KU60019, KU559403, CP466722, caffeine, and wortmannin (see, e.g., Weber et al., “ATM and ATR as Therapeutic Targets in Cancer,” Pharmacol. Ther. 149:124-138 (2015), which is hereby incorporated by reference in its entirety).

In some embodiments, when the phosphatidylinositol 3-kinase-related kinase (PIKK) is ataxia telangiectasia and Rad3-related (ATR), the inhibitor may be an ATR inhibitor selected from the group consisting of schisandrin B, NU6027, NVP-BEZ235, VE821, VE822, AZ20, and AZD6738 (see, e.g., Wagner et al., “Prospects for the Use of ATR Inhibitors to Treat Cancer,” Pharmaceuticals (Basel) 3(5):1311-1334 (2010) and Weber et al., “ATM and ATR as Therapeutic Targets in Cancer,” Pharmacol. Ther. 149:124-138 (2015), which are hereby incorporated by reference in their entirety).

In some embodiments, when the phosphatidylinositol 3-kinase-related kinase (PIKK) is Suppressor of Morphogenesis in Genitalia-1 (SMG-1), the inhibitor may be a SMG-1 inhibitor is selected from the group consisting of miR-192, miR-215, LY294002, and wortmannin (see, e.g., Zhang et al., “SMG-1 Inhibition by miR-192/-215 Causes Epithelial-Mesenchymal Transition in Gastric Carcinogenesis via Activation of Wnt Signaling,” Cancer Med. 7(1):146-156 (2018), which is hereby incorporated by reference in its entirety).

As described supra, treating the subject improves muscle strength, reduces muscle wasting, and/or reduces muscle death in a subject who has a laminopathy affecting cardiace or skeletal muscle. Thus, in some embodiments, administering the PIKK inhibitor improves muscle strength, reduces muscle wasting, and/or reduces muscle cell death. The improvement in muscle strength, reduction in muscle wasting, and/or reduction in muscle cell death may be measured relative to when the PIKK inhibitor is not administered or prior to the administration of the PIKK inhibitor.

As described herein, nuclear damage in LMNA mutant muscle cells can be prevented by microtubule stabilization. Without being bound by theory, it is suggested that nuclear envelope ruptures in LMNA mutant muscle cells result from cytoskeletal forces acting on mechanically weak myonuclei, and that reducing mechanical stress on the nuclei would decrease nuclear damage. Thus, microtubule stabilizing agents may inhibit the progressive nuclear envelope damage associated with the laminopathies described herein.

According to some embodiments, the methods of the present application further involve administering to the subject a microtubule stabilizing agent before, after, or during said administering the inhibitor of a protein associated with a DNA damage response (DDR) pathway.

Suitable microtubule stabilizing agents are well known in the art and include, without limitation, a taxane, an epothilone, discodermolide, sarcodictyin A, sarcodictyin B, eleutherobin, laulimalide, isolaulimalide, peloruside A, and cyclostreptin (see, e.g., Buey et al., “Microtubule Interacations with Chemically Diverse Stabilizing Agents: Thermodynamics of Binding to the Paclitaxel Site Preducts Cytotoxicity,” Chem. Biol. 12(12):1269-1279 (2005), which is hereby incorporated by reference in its entirety). For example, in some embodiments, the microtubule stabilizing agent is a taxane selected from the group consisting of paclitaxel, docetaxel, and abraxane (see, e.g., Castle et al., “Mechanisms of Kinetic Stabilization by the Drugs Paclitaxel and Vinblastine,” Mol. Biol. Cell 28(9):1238-1257 (2017), which is hereby incorporated by reference in its entirety). In other embodiments, the microtubule stabilizing agent is an epothilone selected from the group consisting of epothilone A, epothilone B, epothilone D, aza-epothilone, BMS-310705, KOS-1584, and sagopilone (see, e.g., Buey et al., “Microtubule Interacations with Chemically Diverse Stabilizing Agents: Thermodynamics of Binding to the Paclitaxel Site Preducts Cytotoxicity,” Chem. Biol. 12(12):1269-1279 (2005) and Galmarini, C M., “Sagopilone, a Microtubule Stabilizer for the Potential Treatment of Cancer,” Curr. Opin. Invest. Drugs 10(12):1359-71 (2009), which are hereby incorporated by reference in their entirety).

As described herein, the “nuclear envelope” refers to the selective barrier between the nuclear and cytoplasmic compartment of the cell. In animal cells, the nuclear envelope comprises an inner nuclear membrane (INN), an outer nuclear membrane (ONM), a perinuclear space (PNS) separating the INN and ONM, and the nuclear lamina (Loon et al., “LINC Complexes and Nuclear Positioning,” Semin. Cell. Dev. Biol. 82:67-76 (2018), which is hereby incorporated by reference in its entirety). The major components of the nuclear lamina are the A-type and B-type lamins. The nuclear envelope interacts with the cytoskeleton though a concerved bridge of Sad1p, UNC-84 (SUN)-domain proteins and Klarsicht, ANC-1, Syne Homology (KASH)-domain proteins spans both membranes of the nuclear envelope and is often referred to as the LINC complex because it is the linker of the nucleoskeleton to the cytoskeleton (Tapley et al., “Connecting the Nucleus to the Cytoskeleton by SUN-KASH Bridges Across the Nuclear Envelope,” Curr. Opin. Cell Biol. 25(1):57-62 (2013), which is hereby incorporated by reference in its entirety). To form the bridge, SUN-domain proteins in the INM interact with lamins in the nucleoplasm and KASH-domain proteins in the PNS. KASH-domain proteins are then recruited specifically to the ONM where they are positioned to interact with a wide variety of cytoskeletal components. Thus, SUN-KASH pairs represent links in a molecular chain that spansboth nuclear membranes and which connect nuclear components, including nuclear lamins, to elements of the cytoskeleton (Loon et al., “LINC Complexes and Nuclear Positioning,” Semin. Cell. Dev. Biol. 82:67-76 (2018), which is hereby incorporated by reference in its entirety).

As described herein, agents that block the interaction between SUN-domain and KASH-domain proteins of the LINC complex (i.e., LINC complex disruptors) in LMNA mutant cells substantially reduce DNA-damage, mechanical stress on nuclei, and nuclear damage. Thus, in some embodiments, nuclear damage in LMNA mutant muscle cells can be prevented by disrupting the Linker of Nucleoskeleton and Cytoskeleton (LINC) complex.

According to some embodiments, the methods of the present application further involve administering to the subject a Linker of Nucleoskeleton and Cytoskeleton (LINC) complex disruptor before, after, or during said administering the inhibitor of a protein associated with a DNA damage response (DDR) pathway.

In some embodiments, the LINC complex disruptor selectively targets a Klarsicht, ANC-1, Syne Homology (KASH)-domain protein or a Sad1p, UNC-84 (SUN)-domain protein.

The LINC compex disrupter may be a small molecule, a protein, a peptide, a nucleic acid, an aptamer, an antibody, or a derivative thereof. For example, the LINC complex disruptor may be a nucleic acid molecule selected from a siRNA, shRNA, or miRNA. Methods of generating suitable nucleic acid molecules targeting a protein of the present application (e.g., a protein associated with a DNA damage response pathway, a KASH-domain protein, and/or a SUN-domain protein) are described in detail above. In some embodiments, the disruptor is a selected from the group consisting of an antibody, Fab fragments, F(ab)₂ fragments, Fab′ fragments, F(ab′)₂ fragments, Fd fragments, Fd′ fragments, and Fv fragments.

In some embodiments, the LINC complex disruptor is a dominant negative KASH-domain or a dominant negative SUN-domain. As used herein, a “dominant negative” form of a protein refers to a mutant polypeptide or protein, which lacks wild-type activity and which, once expressed in a cell wherein a wild-type of the same protein is also expressed, dominates the wild-type protein and effectively competes with wild type proteins for substrates, ligands, etc., and thereby inhibits the activity of the wild type molecule. The dominant negative form of a polypeptide or protein includes any polypeptide or representation thereof that differs from a corresponding wild type polypeptide or protein by having at least one amino acid substitution, addition, or deletion.

Suitable domainant negative KASH-domain and SUN-domain inhibitors are well known in the art (Stewart-Hutchinson et al., “Structural Requirements for the Assembly of LINC Complexes and their Function in Cellular Mechanical Stiffness,” Exp. Cell Res. 314:1892-1905 (2008); Lombardi et al., “The Interaction Between Nesprins and Sun Proteins at the Nuclear Envelope Is Critical for Force Transmission Between the Nucleus and Cytoskeleton,” J. Biol. Chem. 286:26743-26753 (2011); and Horn et al., “A Mammalian KASH Domain Protein Coupling Meitotic Chromosomes to the Cytoskeleton,” J. Biol. Chem. 202:1023-1039 (2013), which are hereby incorporated by reference in their entirety) and include those listed in Table 3 below.

TABLE 3
Exemplary KASH-Domain and SUN-Domain Inhibitors

| | | SEQ | |
|---|---|---|---|
| | | ID | Refer- |
| Domain | Amino Acid Sequence | NO: | ence |
| Nesprin1_ | RVLRAALPLQLLLLLLIGLACLVPMSEED | 5 | * |
| HUMAN | YSCALSNNFARSFHPMLRYTNGPPPL | | |
| KASH | | | |
| Domain | | | |
| Nesprin2_ | RVVRAALPLQLLLLLLLLLACLLPSSEED | 6 | * |
| HUMAN | YSCTQANNFARSFYPMLRYTNGPPPT | | |
| KASH | | | |
| Domain | | | |
| Nesprin3_ | RACCVALPLQLLLLLFLLLLFLLPIREED | 7 | * |
| HUMAN | RSCTLANNFARSFTLMLRYNGPPPT | | |
| KASH | | | |
| Domain | | | |
| Nesprin4_ | QPLTFLLILFLLFLLLVGAMFLLPASGGP | 8 | * |
| HUMAN | CCSHARIPRTPYLVLSYVNGLPPV | | |
| KASH | | | |
| Domain | | | |
| ANC1_ | RVLRTALPLQALLVLLMGAACLVPHCDDE | 9 | * |
| CAEEL | YCCQLLNNFAKSFDPSLEFVNGPPPF | | |
| KASH | | | |
| Domain | | | |
| KASH5_ | RVTRHPLIPAPVLGLLLLLLLSVLLLGPS | 10 | * |
| HUMAN | PPPTWPHLQLCYLQPPPV | | |
| KASH | | | |
| Domain | | | |
| UNC83_ | RLIKFTFALSLLAALAAIFYYHVFGKPFG | 11 | * |
| CAEEL | PHVTYVNGPPPV | | |
| KASH | | | |
| Domain | | | |
| Nesprin4_ | LTLFFLLLFLLLVGATLLLPLSGVSCCSH | 12 | ** |
| MOUSE | ARLARTPYLVLSYVNGLPPI | | |
| KASH | | | |
| Domain | | | |
| Sun1 | MDFSRLHTYTPPQCVPENTGYTYALSSSY | 13 | *** |
| Domain | SSDALDFETEHKLEPVFDSPRMSRRSLRL | | |
| | VTTASYSSGDSQAIDSHISTSRATPAKGR | | |
| | ETRTVKQRRSASKPAFSINHLSGKGLSSS | | |
| | TSHDSSCSLRSATVLRHPVLDESLIREQT | | |
| | KVDHFWGLDDDGDLKGGNKAATQGNGELA | | |
| | AEV | | |
| Sun2 | MSRRSQRLTRYSQDDNDGGSSSSGASSVA | 14 | *** |
| Domain | GSQGTVFKDSPLRTLKRKSSNMKHLSPAP | | |
| | QLGPSSDSHTSYYSESVVRESYIGSPRAV | | |
| | SLARSALLDDHLHSEPYVSGDLRGRRRRG | | |
| | TGG | | |

*Jahed et al., “Role of KASH Domain Lengths in the Regulation of LINC Complexes,” MBoC 30(16):1879-2095 (2019), which is hereby incorporated by reference in its entirety.
**Roux et al., “Nesprin 4 is an Outer Nuclear Membrane Protein that can Induce Kinesin-Mediated Cell Polarization,” PNAS USA 106(7):2194-2199 (2009), which is hereby incorporated by reference in its entirety.
***Crisp et al., “Coupling of the Nucleus and Cytoplasm,” J. Cell Biol. 172(1):41-53 (2006), which is hereby incorporated by reference in its entirety.

Another aspect of the present application relates to a method of treating a laminopathy affecting skeletal or cardiac muscle in a subject. This method involves selecting a subject who has a laminopathy affecting skeletal or cardiac muscle; administering, to the selected subject, a microtubule stabilizing agent; and administering, to the selected subject, a Linker of Nucleoskeleton and Cytoskeleton (LINC) complex disruptor before, after, or during said administering the microtubule stabilizing agent to treat the laminopathy affecting skeletal or cardiac muscle in the subject.

Suitable subjects are described supra. In some embodiments, the selected subject is a human. The human may be an adult, a neonate, or a child.

As described in more detail above, the selected subject may have a laminopathy associated with a mutation in one or more genes selected from the group consisting of Lamin A/C (LMNA), emerin (EMD), nesprin-1 (SYNE1), nesprin-2 (SYNE2), SUN domain-containing protein 1 (SUN1), and SUN domain-containing protein 2 (SUN2). The laminopathy may be associated with a mutation in the Lamin A/C (LMNA) gene (NCBI GeneID No: 4000). In some embodiments, the laminopathy may be associated with a mutation in the LMNA gene (NCBI GenelD No: 4000) corresponding to a N195K or H222P substitution in SEQ ID NO: 1. The present application also encompasses mutations in genes selected from the group consisting of EMD (NCBI GeneID No: 2010), SYNE1 (NCBI GeneID No: 23345), SYNE2 (NCBI GeneID No: 23224), SUN1 (NCBI GenelD No: 23353), and SUN2 (NCBI GeneID No: 25777).

Mutations may include a deletion, an insertion, a point mutation, a missense mutation, a frame shift mutation, a truncation, a nonsense mutation, or a splice-site mutation. In some embodiments of the methods described herein, the mutation comprises a non-synonymous single nucleotide base substitution, insertion, or deletion.

In some embodiments, the laminopathy is a striated muscle laminopathy selected from the group consisting of Emery-Dreifuss muscular dystrophy (EDMD), congenital muscular dystrophy, limb-girdle muscular dystrophy type 1B (LGMD1B), dilated cardiomyopathy (DCM), and dilated cardiomyopathy with conduction system defects (DCM-CD). Suitable mutations in LNMA (NCBI GeneID No: 4000) associated with Emery-Dreifuss muscular dystrophy, congenital muscular dystrophy, limb-girdle muscular dystrophy type 1B, dilated cardiomyopathy, and dilated cardiomyopathy with conduction system are described in detail above.

In some embodiments, the microtubule stabilizing agent is a small molecule, a protein, a peptide, a nucleic acid, or an aptamer. Suitable microtubule stabilizing agents are described in more detail above. For example, the microtubule stabilizing agent may be selected from the group consisting of a taxane, an epothilone, discodermolide, sarcodictyin A, sarcodictyin B, eleutherobin, laulimalide, isolaulimalide, peloruside A, and cyclostreptin. In another embodiment, the microtubule stabilizing agent is a taxane selected from the group consisting of paclitaxel, docetaxel, and abraxane. In a further embodiment, the microtubule stabilizing agent is an epothilone selected from the group consisting of epothilone A, epothilone B, epothilone D, aza-epothilone, BMS-310705, KOS-1584, and sagopilone.

In some embodiments, the LINC complex disruptor selectively targets a Klarsicht, ANC-1, Syne Homology (KASH)-domain protein or a Sad1p, UNC-84 (SUN)-domain protein.

As described in more detail above, the LINC compex disrupter may be a small molecule, a protein, a peptide, a nucleic acid, an aptamer, an antibody, or a derivative thereof. For example, the LINC complex disruptor may be a nucleic acid molecule selected from a siRNA, shRNA, or miRNA. Methods of generating suitable nucleic acid molecules targeting a protein of the present application (e.g., a protein associated with a DNA damage response pathway, a KASH-domain protein, and/or a SUN-domain protein) are described in detail above.

In some embodiments, the disruptor is selected from the group consisting of an antibody, Fab fragments, F(ab)₂ fragments, Fab′ fragments, F(ab′)₂ fragments, Fd fragments, Fd′ fragments, and Fv fragments.

As described supra, treating the subject improves muscle strength, reduces muscle wasting, and/or reduces muscle death in a subject who has a laminopathy affecting cardiace or skeletal muscle. Thus, in some embodiments, administering the microtubule stabilizing agent and/or the LINC complex disruptor improves muscle strength, reduces muscle wasting, and/or reduces muscle cell death. The improvement in muscle strength, reduction in muscle wasting, and/or reduction in muscle cell death may be measured relative to when the PIKK microtubule stabilizing agent and/or the LINC complex disruptor is not administered or prior to the administration of the PIKK inhibitor.

Reference to therapeutic agent(s) (i.e., an inhibitor of a protein associated with a DNA damage response (DDR) pathway, a microtubule stabilizing agent, and/or a LINC complex disruptor of the present application) includes any analog, derivative, isomer, metabolite, pharmaceutically acceptable salt, pharmaceutical product, hydrate, N-oxide, crystal, polymorph, prodrug, or any combination thereof.

The therapeutic agent(s) (i.e., an inhibitor of a protein associated with a DNA damage response (DDR) pathway, a microtubule stabilizing agent, and/or a LINC complex disruptor of the present application) may be converted into a salt, if need be, by conventional methods. The term “salt” used herein is not limited as long as the salt is pharmacologically acceptable; preferred examples of salts include a hydrohalide salt (for instance, hydrochloride, hydrobromide, hydroiodide and the like), an inorganic acid salt (for instance, sulfate, nitrate, perchlorate, phosphate, carbonate, bicarbonate and the like), an organic carboxylate salt (for instance, acetate salt, maleate salt, tartrate salt, fumarate salt, citrate salt and the like), an organic sulfonate salt (for instance, methanesulfonate salt, ethanesulfonate salt, benzenesulfonate salt, toluenesulfonate salt, camphorsulfonate salt and the like), an amino acid salt (for instance, aspartate salt, glutamate salt and the like), a quaternary ammonium salt, an alkaline metal salt (for instance, sodium salt, potassium salt and the like), an alkaline earth metal salt (magnesium salt, calcium salt and the like) and the like. In addition, hydrochloride salt, sulfate salt, methanesulfonate salt, acetate salt and the like are preferred as “pharmacologically acceptable salt” of the compounds disclosed herein.

In carrying out the methods of the present application, administering the therapeutic agent(s) (i.e., an inhibitor of a protein associated with a DNA damage response (DDR) pathway, a microtubule stabilizing agent, and/or a LINC complex disruptor of the present application) to a subject may involve administering pharmaceutical compositions containing the agent(s) in therapeutically effective amounts, which means an amount of compound effective in treating the stated conditions and/or disorders in the subject. Such amounts generally vary according to a number of factors well within the purview of ordinarily skilled artisans. These include, without limitation, the particular subject, as well as its age, weight, height, general physical condition, and medical history, the particular compound used, as well as the carrier in which it is formulated and the route of administration selected for it; the length or duration of treatment; and the nature and severity of the condition being treated.

Administering typically involves administering pharmaceutically acceptable dosage forms, which means dosage forms of compounds described herein and includes, for example, tablets, dragees, powders, elixirs, syrups, liquid preparations, including suspensions, sprays, inhalants tablets, lozenges, emulsions, solutions, granules, capsules, and suppositories, as well as liquid preparations for injections, including liposome preparations. Techniques and formulations generally may be found in Remington's Pharmaceutical Sciences, Mack Publishing Co., Easton, Pa., latest edition, which is hereby incorporated by reference in its entirety.

In carrying out methods of the present application, the therapeutic agent(s) (i.e., an inhibitor of a protein associated with a DNA damage response (DDR) pathway, a microtubule stabilizing agent, and/or a LINC complex disruptor of the present application) may be contained, in any appropriate amount, in any suitable carrier substance. The compound may be present in an amount of up to 99% by weight of the total weight of the composition. The composition may be provided in a dosage form that is suitable for the oral, parenteral (e.g., intravenously, intramuscularly), rectal, cutaneous, nasal, vaginal, inhalant, skin (patch), or ocular administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols.

Administering according to the methods of the present application may be carried out systemically or locally. When the administering is carried out systemically into the circulation, the therapeutic agent(s) (i.e., an inhibitor of a protein associated with a DNA damage response (DDR) pathway, a microtubule stabilizing agent, and/or a LINC complex disruptor of the present application) may be formulated for parenteral administration by injection, e.g., by bolus injection or continuous infusion. Solutions or suspensions of the therapeutic agent(s) can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. In all cases, the form must be sterile and must be fluid to the extent that easy syringability exists. It must be stable under the conditions of manufacture and storage and must be preserved against the contaminating action of microorganisms, such as bacteria and fungi.

Administering according to the methods of the present application may be carried out orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes. Thus, in some embodiments, the administering is carried out intramuscularly, intravenously, subcutaneously, orally, or intraperitoneally.

For oral administration, the therapeutic agent(s) (i.e., an inhibitor of a protein associated with a DNA damage response (DDR) pathway, a microtubule stabilizing agent, and/or a LINC complex disruptor of the present application) may be formulated into an inert diluent or an assimilable edible carrier, enclosed in hard or soft shell capsules, compressed into tablets, or incorporated directly into food. The therapeutic agents of the present application may be incorporated with excipients and used in the form of tablets, capsules, elixirs, suspensions, syrups, and the like. Such compositions and preparations should contain at least 0.1% of the therapeutic agent, although lower concentrations may be effective and indeed optimal. The percentage of the therapeutic agent in these compositions may, of course, be varied and may conveniently be between about 2% to about 60% of the weight of the unit.

Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

The therapeutic agent(s) (i.e., an inhibitor of a protein associated with a DNA damage response (DDR) pathway, a microtubule stabilizing agent, and/or a LINC complex disruptor of the present application) may be administered alone or with suitable pharmaceutical carriers, and can be in solid or liquid form, such as tablets, capsules, powders, solutions, suspensions, or emulsions.

Administration of the therapeutic agent(s) described herein (i.e., an inhibitor of a protein associated with a DNA damage response (DDR) pathway, a microtubule stabilizing agent, and/or a LINC complex disruptor of the present application) in the form of a controlled release formulation is especially preferred in cases in which the drug has (i) a narrow therapeutic index (i.e., the difference between the plasma concentration leading to harmful side effects or toxic reactions and the plasma concentration leading to a therapeutic effect is small; in general, the therapeutic index (“TI”) is defined as the ratio of median lethal dose (LD₅₀) to median effective dose (ED₅₀)); (ii) a narrow absorption window in the gastro-intestinal tract; or (iii) a very short biological half-life so that frequent dosing during a day is required in order to sustain the plasma level at a therapeutic level.

Any of a number of strategies can be pursued to obtain controlled release in which the rate of release outweighs the rate of metabolism of the drug in question. Controlled release may be obtained by appropriate selection of various formulation parameters and ingredients, including, e.g., various types of controlled release compositions and coatings. Controlled release formulations include (i) formulations that create a substantially constant concentration of the drug(s) within the body over an extended period of time; (ii) formulations that after a predetermined lag time create a substantially constant concentration of the drug(s) within the body over an extended period of time; (iii) formulations that sustain drug(s) action during a predetermined time period by maintaining a relatively, constant, effective drug level in the body with concomitant minimization of undesirable side effects associated with fluctuations in the plasma level of the active drug substance; (iv) formulations that localize drug(s) action by, e.g., spatial placement of a controlled release composition adjacent to or in the diseased tissue or organ; and (v) formulations that target drug(s) action by using carriers or chemical derivatives to deliver the drug to a particular target cell type.

Suitable regimens for initial administration and further doses or for sequential administrations may all be the same or may be variable. Appropriate regimens can be ascertained by the skilled artisan, from this disclosure, the documents cited herein, and the knowledge in the art.

In some embodiments, the therapeutic agent(s) (i.e., an inhibitor of a protein associated with a DNA damage response (DDR) pathway, a microtubule stabilizing agent, and/or a LINC complex disruptor of the present application) are administered to a subject in one dose. In other embodiments, the therapeutic agent(s) disclosed herein (i.e., the inhibitor of a protein associated with a DNA damage response pathway, the microtubule stabilizing agent, and/or the Linker of Nucleoskeleton and Cytoskeleton complex disruptor of the present application) are administered to a subject in a series of two or more doses in succession. In some other embodiments where the therapeutic agent(s) (i.e., an inhibitor of a protein associated with a DNA damage response (DDR) pathway, a microtubule stabilizing agent, and/or a LINC complex disruptor of the present application) are administered in a single dose, in two doses, and/or more than two doses, the doses may be the same or different, and they are administered with equal or with unequal intervals between them.

The therapeutic agent(s) (i.e., an inhibitor of a protein associated with a DNA damage response (DDR) pathway, a microtubule stabilizing agent, and/or a LINC complex disruptor of the present application) may be administered in many frequencies over a wide range of times. In some embodiments, they are administered over a period of less than one day. In other embodiments, they are administered over two, three, four, five, or six days. In some embodiments, they are administered one or more times per week, over a period of weeks. In other embodiments, they are administered over a period of weeks for one to several months. In various embodiments, they may be administered over a period of months. In others they may be administered over a period of one or more years. Generally, lengths of treatment will be proportional to the length of the disease process, the effectiveness of the therapies being applied, and the condition and response of the subject being treated.

Effective doses of the therapeutic agent(s) (i.e., an inhibitor of a protein associated with a DNA damage response (DDR) pathway, a microtubule stabilizing agent, and/or a LINC complex disruptor of the present application) may vary depending upon many different factors including mode of administration, target site, physiological state of the patient, other medications or therapies administered, and physical state of the patient relative to other medical complications. Treatment dosages need to be titrated to optimize safety and efficacy.

Yet another aspect of the present application relates to a pharmaceutical composition comprising an inhibitor of a protein associated with a DNA damage response (DDR) pathway and a microtubule stabilizing agent.

In some embodiments, the protein associated with a DNA damage response (DDR) pathway is a phosphatidylinositol 3-kinase-related kinase (PIKK). According to some embodiments, the phosphatidylinositol 3-kinase-related kinase (PIKK) is a DNA-dependent protein kinase (DNA-PK), an ataxia telangiectasia mutated serine-protein kinase (ATM), Ataxia Telangiectasia and Rad3 Related (ATR), Suppressor of Morphogenesis in Genitalia-1 (SMG-1), and combinations thereof. For example, the phosphatidylinositol 3-kinase-related kinase (PIKK) may be a DNA-dependent protein kinase and the inhibitor may selectively target a DNA-PK catalytic subunit (DNA-PKcs), Ku70, and/or Ku80.

In some embodiments, the microtubule stabilizing agent is selected from the group consisting of a taxane, an epothilone, discodermolide, sarcodictyin A, sarcodictyin B, eleutherobin, laulimalide, isolaulimalide, peloruside A, and cyclostreptin. According to some embodiments, the microtubule stabilizing agent is a taxane selected from the group consisting of paclitaxel, docetaxel, and abraxane. In other embodiments, the microtubule stabilizing agent is an epothilone selected from the group consisting of epothilone A, epothilone B, epothilone D, aza-epothilone, BMS-310705, KOS-1584, and sagopilone.

In some embodiments, the pharmaceutical composition further includes a Linker of Nucleoskeleton and Cytoskeleton (LINC) complex disruptor. According to some embodiments, the LINC complex disruptor selectively targets a Klarsicht, ANC-1, Syne Homology (KASH)-domain protein or a Sad1p, UNC-84 (SUN)-domain protein. The LINC compex disruptor may be a small molecule, a protein, a peptide, a nucleic acid, or an aptamer. In some embodiments, the LINC complex disruptor is a nucleic acid selected from the group consisting of shRNA, siRNA, and miRNA. In other embodiments, the LINC complex disruptor is selected from the group consisting of an antibody, Fab fragments, F(ab)₂ fragments, Fab′ fragments, F(ab′)₂ fragments, Fd fragments, Fd′ fragments, and Fv fragments.

In certain embodiments, the LINC complex disruptor is a dominant negative KASH domain or a dominant negative SUN domain.

A further aspect of the present application relates to a pharmaceutical composition comprising a Linker of Nucleoskeleton and Cytoskeleton (LINC) complex disruptor and a microtubule stabilizing agent.

Suitable LINC complex disruptors and microtubule stabilizing agents are described in detail above. In some embodiments, the LINC complex disruptor selectively targets a Klarsicht, ANC-1, Syne Homology (KASH)-domain protein or a Sad1p, UNC-84 (SUN)-domain protein.

According to some embodiments, the LINC compex disrupter is a small molecule, a protein, a peptide, a nucleic acid, or an aptamer. The LINC complex disruptor may be a nucleic acid selected from the group consisting of shRNA, siRNA, and miRNA. In some embodiments, the LINC complex disruptor is selected from the group consisting of an antibody, Fab fragments, F(ab)₂ fragments, Fab′ fragments, F(ab′)₂ fragments, Fd fragments, Fd′ fragments, and Fv fragments.

In certain embodiments, the LINC complex disruptor is a dominant negative KASH domain or a dominant negative SUN domain.

Suitable microtubule stabilizing agents are described in detail above. According to some embodiments, the microtubule stabilizing agent is selected from the group consisting of a taxane, an epothilone, discodermolide, sarcodictyin A, sarcodictyin B, eleutherobin, laulimalide, isolaulimalide, peloruside A, and cyclostreptin. In some embodiments, the microtubule stabilizing agent is a taxane selected from the group consisting of paclitaxel, docetaxel, and abraxane. In other embodiments, the microtubule stabilizing agent is an epothilone selected from the group consisting of epothilone A, epothilone B, epothilone D, aza-epothilone, BMS-310705, KOS-1584, and sagopilone.

The pharmaceutical compositions of the present application may comprise a pharmaceutically acceptable carrier.

As described herein, a “pharmaceutically acceptable carrier” refers to a carrier that does not cause an allergic reaction or other untoward effect in patients to whom it is administered and are compatible with the other ingredients in the formulation. Pharmaceutically acceptable carriers include, for example, pharmaceutical diluents, excipients or carriers suitably selected with respect to the intended form of administration, and consistent with conventional pharmaceutical practices. For example, solid carriers/diluents include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof.

Pharmaceutically acceptable carriers may further comprise minor amounts of auxiliary substances such as wetting or emulsifying agents, preservatives or buffers, which enhance the shelf life or effectiveness of the therapeutic agent.

The therapeutic agent(s) (i.e., an inhibitor of a protein associated with a DNA damage response (DDR) pathway, a microtubule stabilizing agent, and/or a LINC complex disruptor of the present application) and/or pharmaceutical compositions disclosed herein can be formulated according to any available conventional method. Examples of preferred dosage forms include a tablet, a powder, a subtle granule, a granule, a coated tablet, a capsule, a syrup, a troche, an inhalant, a suppository, an injectable, an ointment, an ophthalmic ointment, an eye drop, a nasal drop, an ear drop, a cataplasm, a lotion and the like. In the formulation, generally used additives such as a diluent, a binder, an disintegrant, a lubricant, a colorant, a flavoring agent, and if necessary, a stabilizer, an emulsifier, an absorption enhancer, a surfactant, a pH adjuster, an antiseptic, an antioxidant and the like can be used. In addition, the formulation is also carried out by combining compositions that are generally used as a raw material for pharmaceutical formulation, according to conventional methods. Examples of these compositions include, for example, (1) an oil such as a soybean oil, a beef tallow and synthetic glyceride; (2) hydrocarbon such as liquid paraffin, squalane and solid paraffin; (3) ester oil such as octyldodecyl myristic acid and isopropyl myristic acid; (4) higher alcohol such as cetostearyl alcohol and behenyl alcohol; (5) a silicon resin; (6) a silicon oil; (7) a surfactant such as polyoxyethylene fatty acid ester, sorbitan fatty acid ester, glycerin fatty acid ester, polyoxyethylene sorbitan fatty acid ester, a solid polyoxyethylene castor oil and polyoxyethylene polyoxypropylene block co-polymer; (8) water soluble macromolecule such as hydroxyethyl cellulose, polyacrylic acid, carboxyvinyl polymer, polyethyleneglycol, polyvinylpyrrolidone and methylcellulose; (9) lower alcohol such as ethanol and isopropanol; (10) multivalent alcohol such as glycerin, propyleneglycol, dipropyleneglycol and sorbitol; (11) a sugar such as glucose and cane sugar; (12) an inorganic powder such as anhydrous silicic acid, aluminum magnesium silicicate and aluminum silicate; (13) purified water, and the like.

Additives for use in the above formulations may include, for example, (1) lactose, corn starch, sucrose, glucose, mannitol, sorbitol, crystalline cellulose and silicon dioxide as the diluent; (2) polyvinyl alcohol, polyvinyl ether, methyl cellulose, ethyl cellulose, gum arabic, tragacanth, gelatine, shellac, hydroxypropyl cellulose, hydroxypropylmethyl cellulose, polyvinylpyrrolidone, polypropylene glycol-poly oxyethylene-block co-polymer, meglumine, calcium citrate, dextrin, pectin and the like as the binder; (3) starch, agar, gelatine powder, crystalline cellulose, calcium carbonate, sodium bicarbonate, calcium citrate, dextrin, pectic, carboxymethylcellulose/calcium and the like as the disintegrant; (4) magnesium stearate, talc, polyethyleneglycol, silica, condensed plant oil and the like as the lubricant; (5) any colorants whose addition is pharmaceutically acceptable is adequate as the colorant; (6) cocoa powder, menthol, aromatizer, peppermint oil, cinnamon powder as the flavoring agent; (7) antioxidants whose addition is pharmaceutically accepted such as ascorbic acid or alpha-tophenol.

The present application may be further illustrated by reference to the following examples.

### EXAMPLES

The examples below are intended to exemplify the practice of embodiments of the disclosure but are by no means intended to limit the scope thereof.

### Materials and Methods for Examples 1-12

Animals. Lmna KO (Lmna^{-/-}) (Sullivan et al., “Loss of A-type Lamin Expression Compromises Nuclear Envelope Integrity Leading to Muscular Dystrophy,” J. Cell Biol. 147:913-920 (1999), which is hereby incorproated by reference in its entirety), Lmna H222P (Lmna^{H222P/H222P})(Arimura et al., “Mouse Model Carrying H222P-Lmna Mutation Develops Muscular Dystrophy and Dilated Cardiomyopathy Similar to Human Striated Muscle Laminopathies,” Hum. Mol. Genet. 14, 155-169 (2005), which is hereby incorproated by reference in its entirety), and Lmna N195K (Lmna^{N195K/N195K})(Mounkes et al., “Expression of an LMNA-N195K Variant of A-Type Lamins Results in Cardiac Conduction Defects and Death in Mice,” Hum. Mol. Genet. 14, 2167-2180 (2005), which is hereby incorporated by reference in its entirety) have been described previously. Lmna^{+/-}, Lmna^{H222P/+}, and Lmna^{N195K/+} mice were backcrossed at least seven generations into a C57-BL/6 line. For each mouse model, heterozygous mice were crossed to obtain homozygous mutants, heterozygous mice, and wild-type littermates. Lmna mutant mice were provided with gel diet (Nutri-Gel Diet, BioServe) supplement to improve hydration and metabolism upon onset of phenotypic decline. Dmor^{mdx} mice have been described previously (Bulfield et al., “X Chromosome-Linked Muscular Dystrophy (mdx) in the Mouse,” Proc. Natl. Acad. Sci. USA 81:1189-1192 (1984), which is hereby incorporated by reference in its entirety); mice were obtained from the Jackson Laboratory in a C57BL background and hemi- or homozygous animals were bred to produce all hemi- and homozygous offspring. All mice were bred, maintained and euthanized according to IACUC approved protocols. Data from wild-type littermate controls for Lmna KO, Lmna N195K, and Lmna H222P showed no difference in any of the experimental outcomes between the different wild-type littermates, so wild-type data was combined into a single group unless otherwise specified. For both in vivo and in vitro studies, cells and or tissues were isolated from a single mouse and counted as a single replicate. All data are based on at least two independently derived primary cell lines for each genotype.

Nuclear Eenvelope rupture reporter mouse (cGAS/MB21D1-tdTom transgenic mouse). To detect nuclear envelope ruptures in vivo, a transgenic mouse expressing FLAG tagged human cGAS^{E225A/D227A} fused to a tdTomato fluorescent protein (cGAS-tdTomato) under the control of the commonly used constitutive active CMV promoter was generated. The cGAS mutations are in the magnesium-binding domain, abolishing the enzymatic activity and downstream production of interferon, while still retaining the ability to bind to genomic DNA. The mammalian expression cassette including promoter and terminator (CMV-3×FLAG-cGAS^{E225A/D227A}-tdTomato-SV40polyA) was released from the expression vector, removing the prokaryotic domains. The purified linear DNA was then injected into the pronucleus of fertilized embryos collected from super-ovulated C57BL/6 mice and transplanted into pseudo-pregnant recipients. The resulting transgenic mouse model was used to cross into the Lmna KO background to generate 3×FLAG-cGAS^{E225A/D227A}-tdTomato positive Lmna KO mice within two generations.

Myoblast isolation. Cells were harvested from Lmna KO, Lmna N195K, Lmna H222P, and wild-type littermates between 3-5 weeks for Lmna KO mice, 4-6 weeks for Lmna N195K, and 4-10 weeks for Lmna H222P mice using a protocol adapted from Sullivan et al., “Loss of A-type Lamin Expression Compromises Nuclear Envelope Integrity Leading to Muscular Dystrophy,” J. Cell Biol. 147:913-920 (1999), which is hereby incorproated by reference in its entirety. With the exception of the Lmna KO myoblasts, these time-points were prior to the onset of disease phenotypes. Myoblasts from wild-type littermates were harvested at the same time. Muscles of the lower hindlimb were isolated, cleaned of fat, nerve and excess fascia, and kept in HB SS on ice until all mice were harvested. The muscles were digested in 4 ml:1 g of tissue wet weight in a solution of 0.5% Collagenase II (Worthington Biochemicals), 1.2 U/ml Dispase (Worthington Biochemicals), 1.25 mM CaCl₂ (Sigma) in HB SS/25 mM HEPES buffer. Digestion was carried out in a 37° C. water bath for a total time of 60 minutes. At 20 minute intervals, digestion cocktails were removed and triturated 40 times with a 5 ml pipet. In the case of difficult to digest tissues, an extra 25% of 1% Collagenase II was added to the digestion after 40 minutes.

When tissues were fully digested, the reaction was quenched using equal volumes of DMEM supplemented with 10% fetal bovine serum (FBS) and 1% P/S (D10 media, Gibco). The cell suspension was strained through 70 and 40 μm filters (Greiner Bioscience) sequentially to remove undigested myotube fragments and tendon. The cell suspension was centrifuged at 800×g for 5 minutes and washed with 8 ml of D10 media for a total of four times. Cells were then resuspended in primary myoblast growth media (PMGM; Hams F-10 (Gibco) supplemented with 20% horse serum and 1% penicillin/streptomycin and 1 μl/ml basic fibroblast growth factor (GoldBio)) and plated onto a 2% gelatin coated T25 flask. Cells were allowed to sit undisturbed for 72 hours. Capitalizing on the fact that myoblasts adhere much more weakly than fibroblasts, cells were passaged using PBS (calcium- and magnesium-free) instead of trypsin to purify the myoblasts. Cells were washed for 2-3 minutes at room temperature using a volume of PBS sufficient to coat the bottom of the flask and dislodged using manual agitation. When necessary, a 0.000625% trypsin solution was used to aid in the myoblast removal. Myoblasts were re-suspended in PMGM and re-plated onto gelatin coated flasks. This process was continued 3-4 times until pure myoblast cultures were achieved (Springer et al., “Gene Delivery to Muscle,” Current Protocols in Human Genetics,” Chapter 13: Unit 14 (2002), which is hereby incorporated by reference in its entirety). Cells were maintained in culture on gelatin coated flasks with media changes every other day. All experiments were carried out prior to passage 12. Each independent experiment was done on a different set of lamin mutant and wild-type littermates such that each independent experiment was sourced from a different animal to account for heterogeneity in phenotype.

Myoblast differentiation. Myoblasts were differentiated according to a protocol modified from Pimentel et al., “In Vitro Differentiation of Mature Myofibers for Live Imaging,” Journal of Visualized Experiments: JoVE (2017), which is hereby incorporated by reference in its entirety. Coverslips for differentiation were prepared by first coating with CellTak (Corning) according to the manufacturer's protocol and then coating with growth factor reduced Matrigel (Corning) diluted 1:100 with IMDM with Glutamax (Gibco). Pre-cooled pipette tips were used to avoid premature polymerization. Matrigel was allowed to polymerize at 37° C. for 1 hour and the excess solution was aspirated. Primary myoblasts were seeded at a density of 55,000 cells/cm² in PMGM. Cells were allowed to attach for 24 hours before being switched to primary myoblast differentiation media (PMDM) composed of IMDM with Glutamax and 2% horse serum without antibiotics. This timepoint was considered day 0. One day after the onset of differentiation, a top coat of 1:3 Matrigel:IMDM was added to the cells and allowed to incubate for 1 hour at 37° C. PMDM supplemented with 100 ng/ml agrin (R&D Systems) was added to the cells and henceforth replaced every second day. Cells were allowed to differentiate for a total of 0, 5, or 10 days.

Plasmids and generation offluorescently labeled cell lines. Each of the mutant myoblast lines were stably modified with lentiviral vectors to express the nuclear rupture reporter NLS-GFP (pCDH-CMV-NLS-copGFP-EF1-blastiS) and cGAS-mCherry (pCDH-CMV-eGAs^{E225A/D227A}-mCherry2-EF1-blastiS). cGAS is a cytosolic DNA binding protein; a cGAS mutant (E225A/D227A) with abolished enzyme activity and interferon production, but that still binds DNA (Civril et al., “Structural Mechanism of Cytosolic DNA Sensing by cGAS,” Nature 498:332-337 (2013), which is hereby incorporated by reference in its entirety) and serves as a nuclear envelope rupture reporter (Denais et al., “Nuclear Envelope Rupture and Repair During Cancer Cell Migration,” Science 352:353-358 (2016), which is hereby incorporated by reference in its entirety) was used. For rescue experiments, Lmna KO cells were modified with human lamin A (pCDH-CMV-preLamin A-IRES-GFP-puro). To generate the DN-KASH and DN-KASHext constructs, GFP-KASH2 and GFP-KASH2ext were subcloned from previously published plasmids (Stewart-Hutchinson et al., “Structural Requirements for the Assembly of LINC Complexes and their Function in Cellular Mechanical Stiffness,” Exp. Cell Res. 314:1892-1 905 (2008), which is hereby incorporated by reference in its entirety) and inserted into an all-in-one doxycycline inducible backbone (pPB-tetO-GFP-KASH2-EIF1α-rtTA-IRES-Neo) (Shurer et al., “Physical Principles of Membrane Shape Regulation by the Glycocalyx,” Cell 177:1757-1770 e1721 (2019), which is hereby incorporated by reference in its entirety).

Viral and Piggybac modification. Pseudoviral particles were produced as described in Denais et al., “Nuclear Envelope Rupture and Repair During Cancer Cell Migration,” Science 352:353-358 (2016), which is hereby incorporated by reference in its entirety). In brief, 293-TN cells (System Biosciences, SBI) were co-transfected with the lentiviral-containing, packaging and envelope plasmids using PureFection (SBI), following manufactures protocol. Lentivirus-containing supernatants were collected at 48 hours and 72 hours after transfection, and filtered through a 0.45 μm filter. Cells to be transduced were seeded into 6-well plates so that they reached 50-60% confluency on the day of infection and transduced at most 2 consecutive days with the viral supernatant using the TransDux Max system (SBI). The viral solution was replaced with fresh culture medium, and cells were cultured for 72 hours before selection with 1 μg/mL of puromycin or 2 μg/mL blasticidin S for 2-5 days. After selection, cells were subcultured and maintained in their recommended medium without the continued use of selection agents. For PiggyBac modifications, myoblasts were transfected with 1.75 μg of the PiggyBac plasmid and 0.75 μg of a Hyperactive Transposase using the Lipofectamine 3000 reagent according to the manufacture's guidelines.

Extended imaging using incubator microscope. Long term imaging was performed using an Incucyte imaging system, which allows for incubator imaging to minimize the effects of humidity and CO₂ changes. The differentiating cells expressing combinations of NLS-GFP and cGAS-mCherry were imaged using the Incucyte dual color filter module from day 0 to day 10, every 30-60 minutes with a 20× objective. Resulting images were analyzed using the Incucyte software, which performs fluorescence background subtraction using a top hat method and then subsequent thresholding. cGAS-mCherry cells were thresholded and then analyzed for increase in fluorescent foci over time to track the rate of increase in nuclear envelope rupture or damage. NLS-GFP cells were used to investigate the frequency and presence of nuclear envelope rupture. To verify the results obtained from the Incucyte, cells were fixed and stained with appropriate antibodies to evaluate DNA damage and nuclear envelope rupture.

Isolation of single muscle fibers. Single muscle fibers were harvested in a protocol adapted from Vogler et al., “Isolation, Culture, Functional Assays, and Immunofluorescence of Myofiber-Associated Satellite Cells,” Methods in Molecular Biology 1460:141-162 (2016), which is hereby incorporated by reference in its entirety. Briefly, fibers were isolated from the extensor digitorum longus (EDL) of male and female Lmna KO and wild-type litter mates at 5-6 weeks of age and Lmna H222P and wild-type litter mates were harvested at 6-8 weeks of age at 23-25 weeks of age in order to compare pre- and post-phenotype onset tissue (Sullivan et al., “Loss of A-type Lamin Expression Compromises Nuclear Envelope Integrity Leading to Muscular Dystrophy,” J. Cell Biol. 147:913-920 (1999); Arimura et al., “Mouse Model Carrying H222P-Lmna Mutation Develops Muscular Dystrophy and Dilated Cardiomyopathy Similar to Human Striated Muscle Laminopathies,” Hum. Mol. Genet. 14, 155-169 (2005); and Arimura et al., “Nuclear Accumulation of Androgen Receptor in Gender Difference of Dilated Cardiomyopathy due to Lamin A/C mutations,” Cardiovasc Res 99:382-394 (2013), which are hereby incorporated by reference in their entirety). Briefly, the EDL (extensor digitorus longus) and plantaris were isolated from the mouse and placed directly into a 1 ml solution of F10 media with 4,000 U/ml of Collagenase I (Worthington Biochemicals). The tissue was digested for 15-40 minutes depending on muscle size in a 37° C. water bath with agitation by inversion every 10 minutes. The reaction was quenched by transferring the digestion mixture to 4 ml of PMGM. Single fibers were hand-picked from the digested tissue using fire polished glass Pasteur pipettes. When necessary, the tissue was further dissociated by manual pipetting with a large-bore glass pipet. Fibers were washed once in fresh media prior to fixation with 4% paraformaldehyde (PFA) for 15 minutes at room temperature and subsequent IF staining.

Pharmacological treatments. For preliminary experiments, myoblasts were differentiated using the standard protocol and treated with pharmacological treatments starting at day 5 of differentiation. For chromatin protrusion studies, paclitaxel was administered to differentiated myotubes in two 24 hours bursts at day 4 and day 6-post differentiation with a 24 hour recovery in between. Myotubes were then fixed in 4% PFA at day 7 and stained with anti-lamin B and DAPI in order to quantify the percentage of myonuclei with chromatin protrusions. For long term studies using the cGAS reporter, myotubes were treated with 10 nM of paclitaxel starting at day 5 and then media was refreshed every day. To inhibit myotube contraction, cells were treated with 5 μM nifedipine starting at day 5 and then media was refreshed every day. For DNA damage induction and inhibitor experiments, cells were treated with 20 μg/ml of phleomycin for a two-hour pulse on Day 3, 4, and 5 of differentiation. Concurrently, cells were treated with NU7441 (2 μM), KU55933 (2 μM) starting at day 2 of differentiation through day 10 of differentiation. For induction of DN-KASH and DN-KASHext, cells were treated with 1 μM doxycycline.

Biophysical assays. To evaluate nuclear deformability in high throughput, a microfluidic, micropipette aspiration device was designed and fabricated. The mask and wafers were produced in the Cornell NanoScale Science and Technology Facility (CNF) using standard lithography techniques. PDMS molds of the devices were cast using Sylgard 184 (Dow Corning) and mounted on coverslips using a plasma cleaner as described previously (Denais et al., “Nuclear Envelope Rupture and Repair During Cancer Cell Migration,” Science 352:353-358 (2016), which is hereby incorporated by reference in its entirety). Three port entrances were made using a 1.2 mm biopsy punch. Pressures at the inlet and outlet ports were set to 1.0 and 0.2 psi (relative to atmospheric pressure, Pₐₜₘ), respectively, using compressed air regulated by a MCFS-EZ pressure controller (Fluigent) to drive single cells through the device. Myoblasts (˜5×10⁶ cells/mL suspended in 2% bovine serum albumin (BSA), 0.2% FBS and 10 μg/mL Hoechst 33342 DNA stain in PBS) were captured within an array of 18 pockets, and then forced to deform into 3 μm wide×5 μm tall micropipettes. The selected pressures resulted in detectable nuclear deformations without causing significant damage to the cells (tested using propidium iodide staining). The remaining port was set to Pₐₜₘ and outfitted with a handheld pipette to flush cells from the pockets at the start of each image acquisition sequence. Brightfield and fluorescence images were acquired every 5 seconds for a minimum of 60 seconds using an inverted microscope and 20×/NA 0.8 air objective. Nuclear protrusion length was calculated using a custom-written MATLAB program, made available upon request.

For the microharpoon studies, myoblasts were seeded in 35 mm glass bottom dishes and differentiated as previously described, except without the addition of a Matrigel top coat to allow microharpoon access. A Sutter P-97 micropipette puller was used to create microharpoons from borosilicate glass rods (Sutter; OD: 1.0 mm, ID: 0.78, 10 cm length) with tip diameters of ≈1 μm. Day 4 myotubes (Lmna KO and wild-type) were treated for 24 hours with either 50 nM Paclitaxel or the corresponding 0.1% DMSO. The following day, the microharpoon assay was performed as described in Fedorchak et al., “Cell Microharpooning to Study Nucleo-Cytoskeletal Coupling,” Methods Mol. Biol. 1411:241-254 (2016), which is hereby incorproated by reference in its entirety, with slight modifications to the pull parameters to accommodate myotubes. The microharpoon was inserted ≈5-7 μm from the edge of the nucleus and pulled 15 μm at a rate of 1 μm/s. Pull direction was always orthogonal to the long axis of the myofiber. Images were acquired at 40× (+1.6×) every 5 seconds. Nuclear strain and centroid displacement were calculated using a custom-written MATLAB program.

siRNA treatment. siRNAs used were as follows: Kif5b#3 (target sequence 5′-CAGCAAGAAGTAGACCGGATA-3′ (SEQ ID NO:15); Qiagen SI00176050), Kif5b#4 (target sequence 5′-CACGAGCTCACGGTTATGCAA-3′ (SEQ ID NO:16); Qiagen SI00176057), and non-target (NT) negative control (ON-TARGETplus non-targeting pool, Dharmachon, D-001810-10). Myoblasts were seeded at a density of ˜15,000 cells per well in a 96-well glass bottomed dish containing a matrigel coating. Once adhered, the myoblasts were transfected twice, 48 hours apart, with siRNA for NT or Kif5b using Lipofectamine RNAiMAX at a concentration of 150 nM in PMGM. After 12 hours, the myoblasts were switched to PMDM and differentiated for 5 days.

Immunofluorescence staining of mouse cells and tissues. Cells were fixed in pre-warmed 4% PFA at the appropriate time point(s) and washed with PBS. Cells were blocked and permeabilized with a solution of 3% BSA, 0.1% Triton-X 100 and 0.1% Tween (Sigma) for 1 hour at room temperature. Cells were stained with primary antibodies diluted in blocking solution according to Table 4 at 4° C. overnight. Samples were washed with PBS and incubated for 1 hour at room temperature with 1:250 dilution of AlexaFluor antibodies (Invitrogen) and 1:1000 DAPI (Sigma). Single muscle fibers were stained using the same procedure in Eppendorf tube baskets with an increase in blocking solution Triton-X concentration to 0.25%.

TABLE 4
Primary Antibodies and Corresponding Dilutions for
Immunofluorescence Stanining and Western Blotting

| Antibody | Cat# | Vendor | Dilution |
|---|---|---|---|
| MyHC | A4.1025 | DSHB | 1:100 |
| MyHC | MAB4470-SP | Novus | 1:500 |
| | | Biologicals | |
| Lamin B (M-20) | sc-6217 | Santa Cruz | 1:200 |
| Lamin B1 (B-10) | sc-374015 | Santa Cruz | 1:200 |
| Lamin A (H-102) | sc-20680 | Santa Cruz | 1:200 |
| Lamin A/C (E1) | sc-376248 | Santa Cruz | 1:200 |
| Gamma-H2AX (Ser139) | 80312 | Cell Signaling | 1:200 |
| Gamma-H2AX (Ser139) | 9718 | Cell Signaling | 1:200 |
| HSP90 α/β (F-8) | sc-13119 | Santa Cruz | 1:200 |
| Nesprin1-E | MANNES1E | Glen Morris | 1:500 |
| Nesprin-1-A | MANNES1A | Glen Morris | 1:500 |
| alpha-tubulin | T9026 | Sigma | 1:500(IF) |
| | | | 1:5000(WB) |
| NPC (414) | Ab50008 | Abcam | 1:500 |
| Emerin | NCL-EMERIN | Leica | 1:200 |
| DNA-PKcs (S2056) | ab18192 | Abcam | 1:1000 |
| DNA-PKcs | sc-390849 | Santa Cruz | 1:750 |
| Cleaved Caspase-3 | 9661 | Cell Signaling | 1:500 |
| 53BP1 | NB100-304 | Novus | 1:1000 |
| | | Biologicals | |
| Dystrophin | Mab7A10 | University of | 1:20 |
| | | Iowa Hospitals | |
| | | and Clinics | |
| | | Pathology Core | |

Human patient biopsy staining. Following diagnostic testing, muscle biopsies were stored at −80° C. and subsequently utilized for research following protocols approved by the corresponding IRB, with informed consent from all participants. Cryopreserved human quadriceps muscle biopsy tissue from LMNA muscular dystrophy individuals and age-matched controls were used for immunostaining as described (Dialynas et al., “Myopathic Lamin Mutations Cause Reductive Stress and Activate the nrf2/keap-1 Ppathway,” PLoS Genet 11:e1005231 (2015), which is hereby incorporated by reference in its entriety). An anti-rabbit polyclonal 53BP1 antibody (Novus) and anti-dystrophin mouse monocolonal antibody (Mab7A10, U of Iowa Hospitals and Clinics Pathology Core) were used at 1:1000 and 1:20 dilutions, respectively. Texas Red labeled phalloidin (Invitrogen) was used at 1:400 dilution. Secondary antibodies were a goat anti-rabbit Ig Alexa 488 conjugate (Invitrogen) and a goat anti-mouse IgG rhodamine Red-X conjugated (Molecular Probes), both used at 1:500 dilution. Slides were imaged on a Zeiss 710 confocal microscope (University of Iowa Central Microscopy Facility). The intensity of nuclear anti-53BP1 staining was quantified using ImageJ. Analysis of the human muscle samples was performed in a double-blinded manner. A pathologist generated the 10 μm thick cryo-sections and coded the samples, which were stained, imaged, and quantified prior to decoding by an independent individual.

Western analysis. Cells were lysed in RIPA buffer containing protease (cOmplete EDTA-Free, Roche) and phosphatase (PhosSTOP, Roche) inhibitors. Protein was quantified using Bio-Rad Protein Assay Dye and 25-30 μg of protein lysate was separated using a 4-12% Bis-Tris polyacrylamide gel using standard a standard SDS-Page protocol. Protein was transferred to a polyvinylidene fluoride (PVDF) membrane overnight at 4° C. at a current of 40 mA. Membranes were blocked using 3% BSA in tris-buffered saline containing 0.1% Tween-20 and primary antibodies (Table 4) were diluted in the same blocking solution and incubated overnight at 4° C. Protein bands were detected using either IRDye 680LT or IRDye 800CW (LI-COR) secondary antibodies, imaged on an Odyssey® CLx imaging system (LI-COR) and analyzed in Image Studio Lite (LI-COR).

Imaging acquisition. Cells on coverslips and mounted single muscle fibers were imaged with an inverted Zeiss LSM700 confocal microscope. Z-stack were collected using 20× air (NA=0.8), 40× water-immersion (NA=1.2) and 63× oil-immersion (NA=1.4) objectives. Airy units for all images were set between 1 and 1.5. Epi-fluorescence images were collected on a motorized inverted Zeiss Observer Z1 microscope equipped with CCD cameras (Photometrics CoolSNAP EZ or Photometrics CoolSNAP KINO) or a sCMOS camera (Hamamatsu Flash 4.0). H&E histology images were collected on an inverted Zeiss Observer Z1 microscope equipped with a color CCD camera (Edmund Optics, EO-0312C).

Image analysis. Image sequences were analyzed using ZEN (Zeiss), ImageJ, or MATLAB (Mathworks) using only linear adjustments uniformly applied to the entire image region. Region of interest intensities were extracted using ZEN or ImageJ. To quantify cleaved caspase-3 (i.e. active) area and myofiber health, maximum intensity protections were generated, which were then blinded to the observer. Cleaved caspase-3 area was calculated by thresholding of the caspase-3 and myosin heavy chain fluorescent signal and expressing the cleaved caspase-3 signal relative to the myosin heavy chain signal. Myofiber contractions were scored based on a minimum of 6 random fields of view per replicate using a blinded analysis according to the scales provided in FIGS. 4A-4B. To count the number of DNA protrusions, and DNA damage foci, confocal image stacks were three-dimensionally reconstructed and displayed as maximum intensity projections. Protrusions lengths were both counted and measured by the presence of DAPI signal beyond the lamin B rim of the nucleus. Aspect ratio was quantified based on a thresholded lamin B rim to avoid the confounding factor of the DNA protrusions outside the body of the nucleus. Nuclear rupture was detected by an increase of the cytoplasmic NLS-GFP signal, or the localization of cGAS-mCherry to the nucleus. For better visualization of NLS-GFP cells many of the fluorescent single color image sequences were inverted. Graphs were generated in Excel (Microsoft), and figures were assembled in Illustrator (Adobe). DNA damage was determined by counting H2AX foci and then binned based on foci number. If damage was so severe that individual foci could not be counted, these nuclei were placed in the >25 foci category. For Hsp90 quantification, average nuclear Hsp90 fluorescence intensity was determined from a single mid-nucleus z-plane image and normalized to the cytoplasmic intensity at two points immediately adjacent to the nucleus.

MTT assay. Myoblasts, seeded in a 96-well plate and differentiated as previously described for 0, 5, or 10 days, were assayed for cell viability according to the manufacturer's instructions (Promega, CellTiter 96 Non-Radioactive Cell Proliferation Assay). Fresh differentiation media was added two hours prior to the addition of 15 μL MTT 3-(4,5-dimethylthiazol-2-yl)-2,5-diphenyltetrazolium bromide dye. After incubation for 3 hours in MTT dye, 100 uL of Stop Solution was added to solubilize the formazan product (appears purple). Following overnight incubation at 37° C. and 5% CO₂, the absorbance of each well (measured at 590 nm) was analyzed using a microplate reader.

Gamma irradiation. A pulse of gamma-irradiation (5 Gy) was administered to myotubes differentiating (5 days) in a 96 well plate. Non-irradiated controls, along with treated cells following 3, 6, or 24 hours of recovery, were PFA-fixed and stained with anti-γH2AX, anti-lamin B and DAPI. A custom macro was used to quantify the mean integrated density of nuclear γH2AX signal from maximum intensity projections of confocal z-stacks.

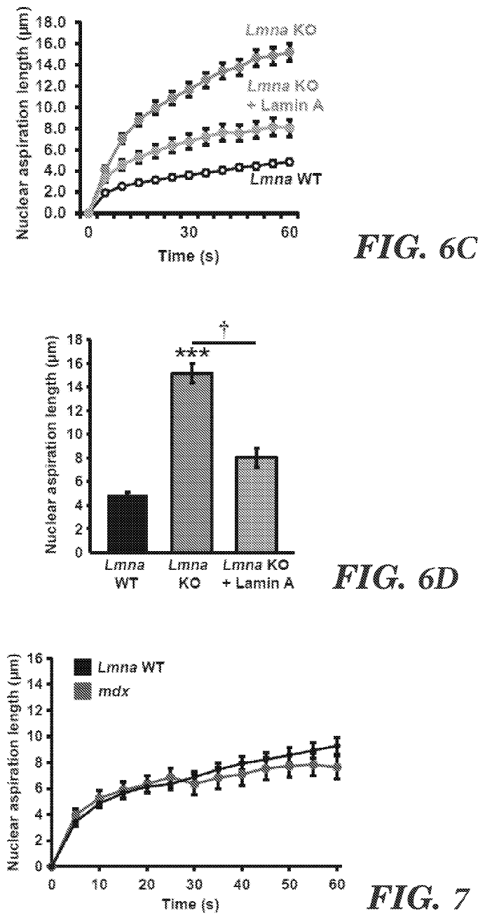

Statistical analysis. Unless otherwise noted, all experimental results were taken from at least three independent experiments and in vivo data were taken from at least three animals. For data with normal distribution, either student's t-tests (comparing two groups) or one-way ANOVA (for experiments with more than two groups) with post-hoc tests were used. When multiple comparisons were made, the significance level was adjusted using Bonferroni corrections. All tests were performed using GraphPad Prism. Micropipette aspiration data were natural log-transformed (FIG. 6A) and analyzed by linear regression of the log-log data. In addition, data was analyzed with a multilevel model, in which the log-transformed protrusion length was the dependent variable in the model and the log-transformed time, genotype, and their interaction were treated as independent fixed effects. Variance from individual experiments and other effects were considered in the model as random effects. Post-hoc multiple comparisons test with Dunnett correction were performed to determine differences between Lmna mutant cells (Lmna KO, Lmna N195K, and Lmna H222P) and control cells (pooled wild-type). Analyses were carried out using JMP software. For human tissue samples, nuclei were binned into eleven categories based on their intensity of 53BP1 staining as measured with ImageJ (arbitrary units). The relative percent intensities of each bin were plotted as a histogram for each genotype. Since the intensity values were not in a normal distribution, the Kruskal-Wallis one-way analysis of variance (ANOVA), followed by the Dunn post hoc test, were used to determine if there was a significant difference in staining among the genotypes. The Mann-Whitney non-parametric test was used for comparisons that involved one sample and one age-matched control. Unless otherwise noted, * denotes p≤0.05, ** denotes p≤0.01, and *** denotes p≤0.001. Unless otherwise indicated, error bars represent the standard error of the mean (SEM). The data that support the findings of this study are available from the corresponding author upon reasonable request.

### Example 1

### Lmna Mutations Cause Progressive Decline in Myofiber Health In Vitro and In Vivo

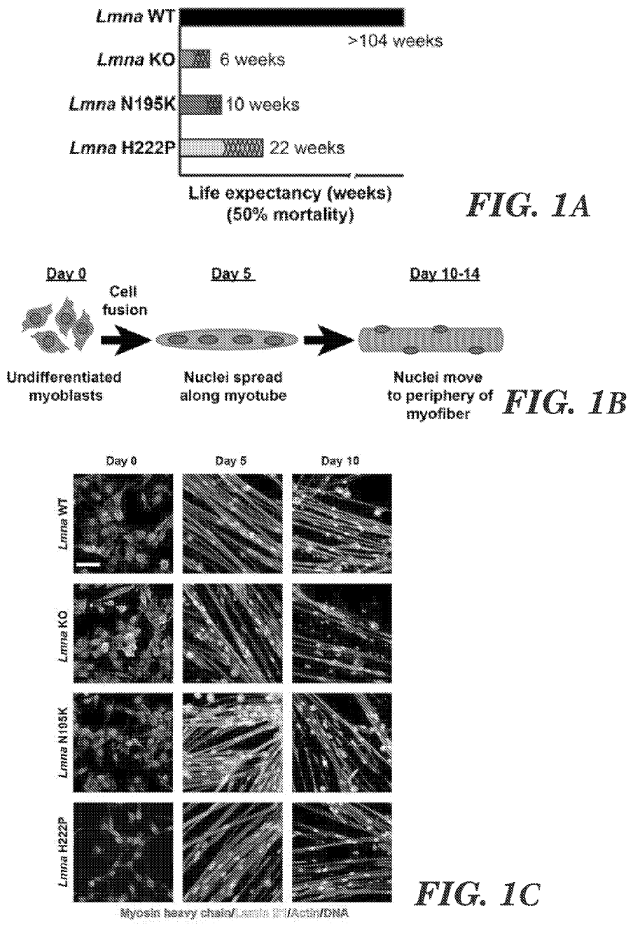

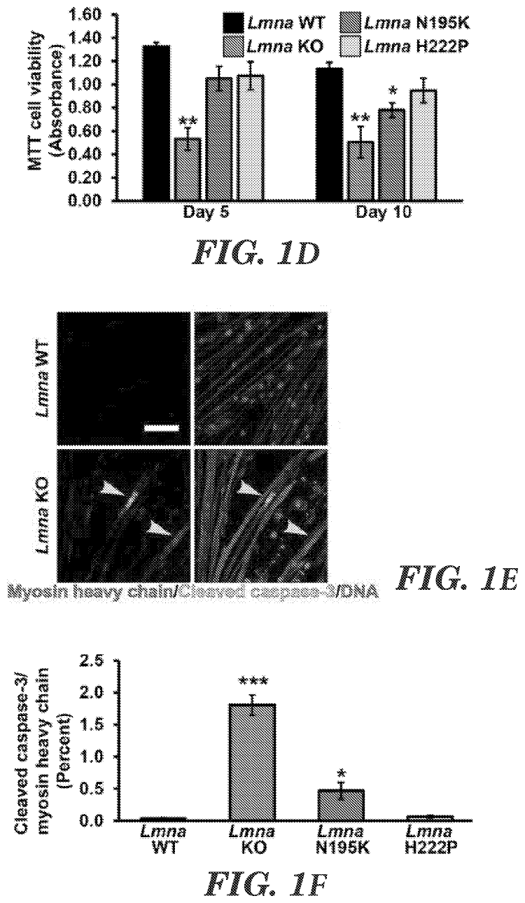

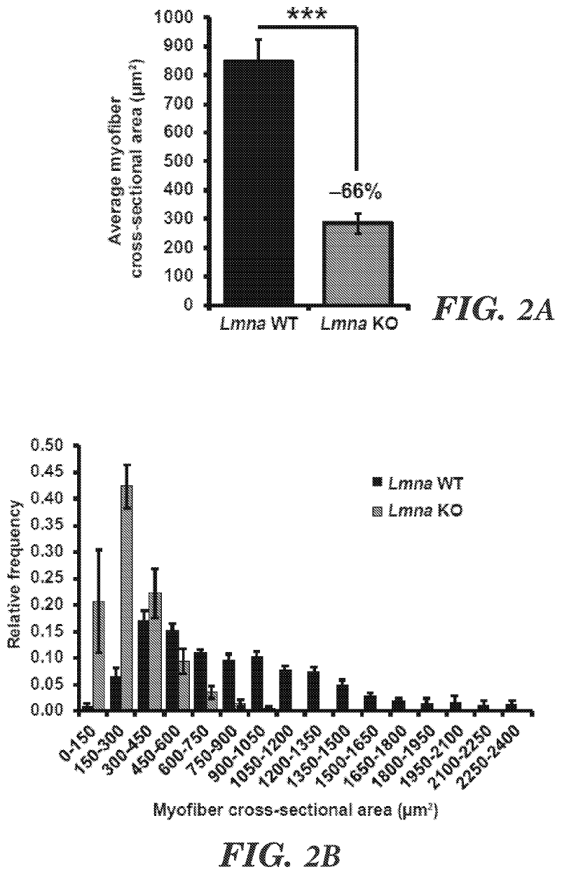

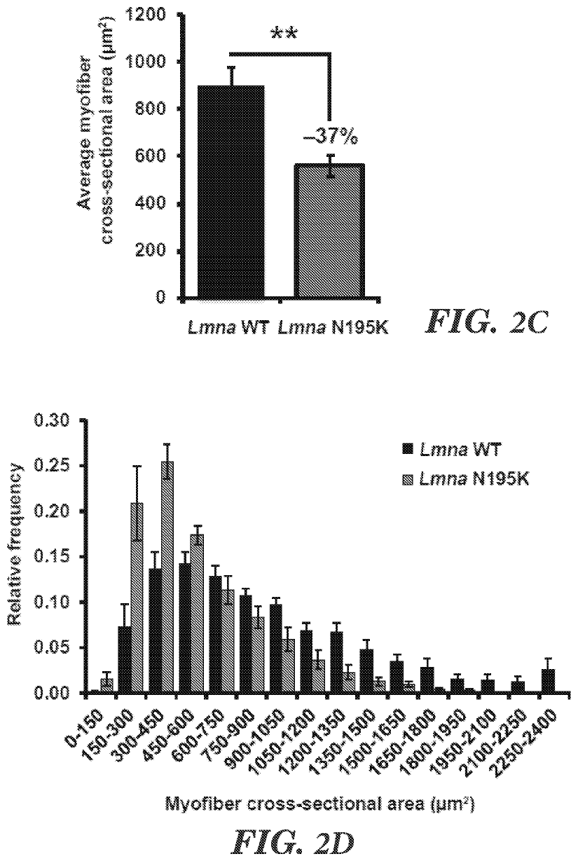

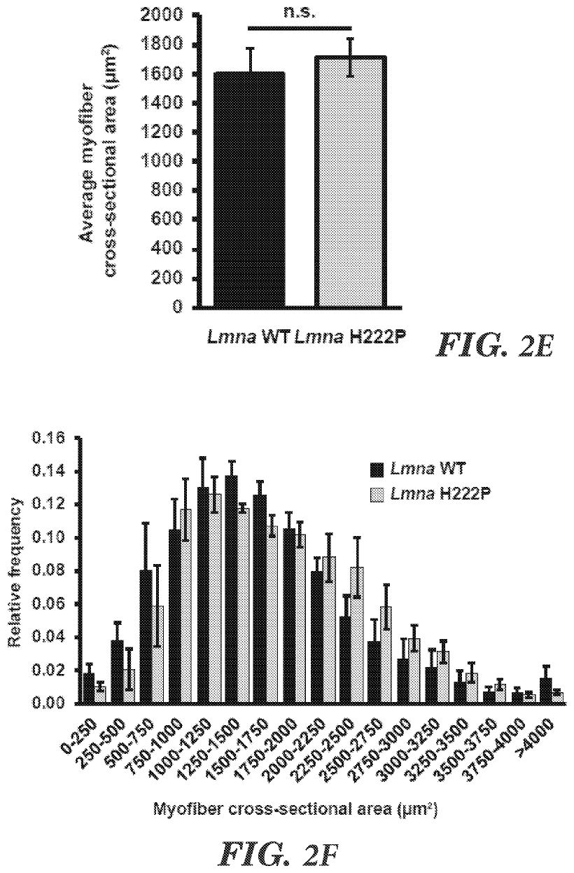

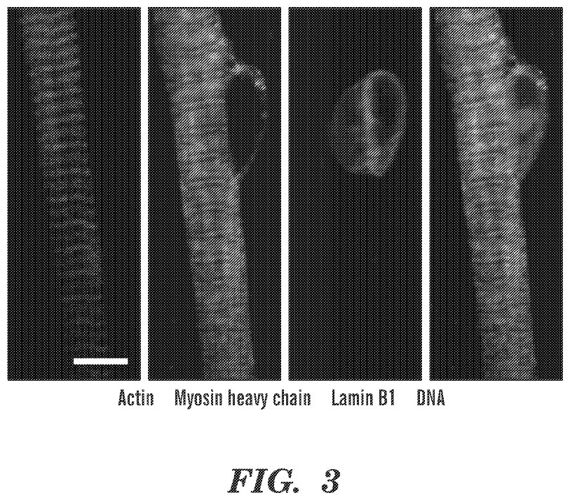

To examine the effect of Lmna mutations on nuclear mechanics and muscle function in vitro, myoblasts from three established mouse models of striated muscle laminopathies, representing a spectrum of muscle wasting and disease severity, were isolated (FIGS. 1A, 2A): Lamin A/C-deficient (Lmna^{-/-}) mice (Sullivan et al., “Loss of A-type Lamin Expression Compromises Nuclear Envelope Integrity Leading to Muscular Dystrophy,” J. Cell Biol. 147:913-920 (1999), which is hereby incorproated by reference in its entirety), subsequently referred to as lamin A/C knock-out mice (Lmna KO); knock-in mice carrying the Lmna^{N195K/N195K} mutation (Lmna N195K) (Mounkes et al., “Expression of an LMNA-N195K Variant of A-Type Lamins Results in Cardiac Conduction Defects and Death in Mice,” Hum. Mol. Genet. 14, 2167-2180 (2005), which is hereby incorporated by reference in its entirety); knock-in mice carrying the Lmna^{H222P/H222P} mutation (Lmna H222P) (Arimura et al., “Mouse Model Carrying H222P-Lmna Mutation Develops Muscular Dystrophy and Dilated Cardiomyopathy Similar to Human Striated Muscle Laminopathies,” Hum. Mol. Genet. 14, 155-169 (2005), which is hereby incorproated by reference in its entirety); and wild-type littermates (FIGS. 1A, 2A-2F). While the Lmna N195K mice were originally described as a model for dilated cardiomyopathy (Mounkes et al., “Expression of an LMNA-N195K Variant of A-Type Lamins Results in Cardiac Conduction Defects and Death in Mice,” Hum. Mol. Genet. 14, 2167-2180 (2005), which is hereby incorporated by reference in its entirety), in the C57BL/6 background used in the studies described herein, the mice developed pronounced skeletal muscular dystrophy in addition to cardiac defects (FIGS. 2A-2F). For in vitro studies, a recently developed, three-dimensional culture protocol was utilized to differentiate primary myoblasts into mature, contractile myofibers over the course of ten days (FIG. 1B) (Pimentel et al., “In Vitro Differentiation of Mature Myofibers for Live Imaging,” Journal of Visualized Experiments: JoVE (2017) and Roman et al., “An In Vitro System to Measure the Positioning, Stiffness, and Rupture of the Nucleus in Skeletal Muscle,” Methods Mol. Biol. 1840:283-293 (2018), which are hereby incorproated by reference in their entirety). The resulting myofibers display the highly organized sarcomeric structure and evenly spaced peripheral myonuclear positioning characteristic of mature skeletal muscle fibers in vivo (FIG. 3).

All myoblasts, including the Lmna KO, Lmna N195K and Lmna H222P mutant cells, successfully completed myoblast fusion, differentiated into myotubes, and matured into myofibers (FIG. 1C), consistent with previous studies on differentiation of Lmna KO myoblasts

(Melcon eg al., “Loss of Emerin at the Nuclear Envelope Disrupts the Rb1/E2F and MyoD Pathways during Muscle Regeneration,” Hum. Mol. Genet. 15:637-651 (2006) and Cohen et al., “Defective Skeletal Muscle Growth in Lamin A/C-Deficient Mice is Rescued by loss of Lap2α,” Human Mol. Gen. 22(14):2852-2869 (2013), which are hereby incorporated by reference in their entirety). Wild-type myofibers remained healthy and highly contractile up to ten days of differentiation. In contrast, the Lmna KO myofibers showed a decline in cell contractility, viability, and number of myonuclei, starting at day five of differentiation (FIGS. 1C-1D. 4A-4B). The Lmna N195K myofibers showed a similar, albeit slightly delayed decline in cell viability, contractility, and number of myonuclei by day ten of differentiation (FIGS. 1D, 4A-4B). The reduction in cell viability at day ten in the Lmna KO and Lmna N195K myofibers was associated with an increase in activated caspase-3 (FIGS. 1E-1F), indicating that reduced viability was due at least in part to cell-intrinsic apoptosis. Unlike the Lmna KO and Lmna N195K models, the Lmna H222P myofibers exhibited no significant decrease in viability, contractility, or number of myonuclei (FIGS. 1D, 4A-4B) within ten days of differentiation. Taken together, the long-term differentiation assays revealed a striking correlation among the defects observed in vitro, including loss of muscle cell viability and presence of apoptotic markers, with the severity of the disease in the corresponding mouse models (FIGS. 1A, 2A), suggesting that defects in the in vitro model may serve as prognostic markers for disease progression.

### Example 2

### Lmna Mutant Muscle Cells Have Reduced Nuclear Stability that Corresponds to Disease Severity

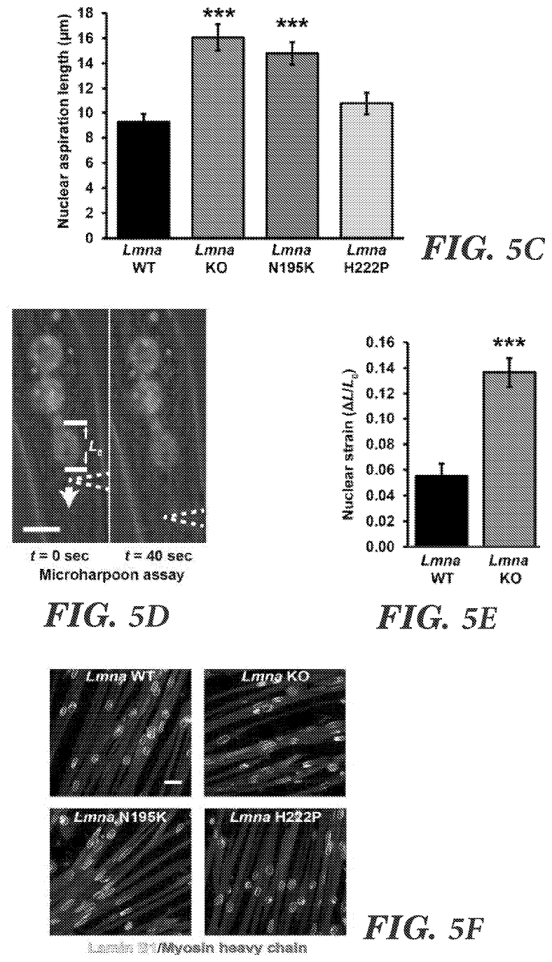

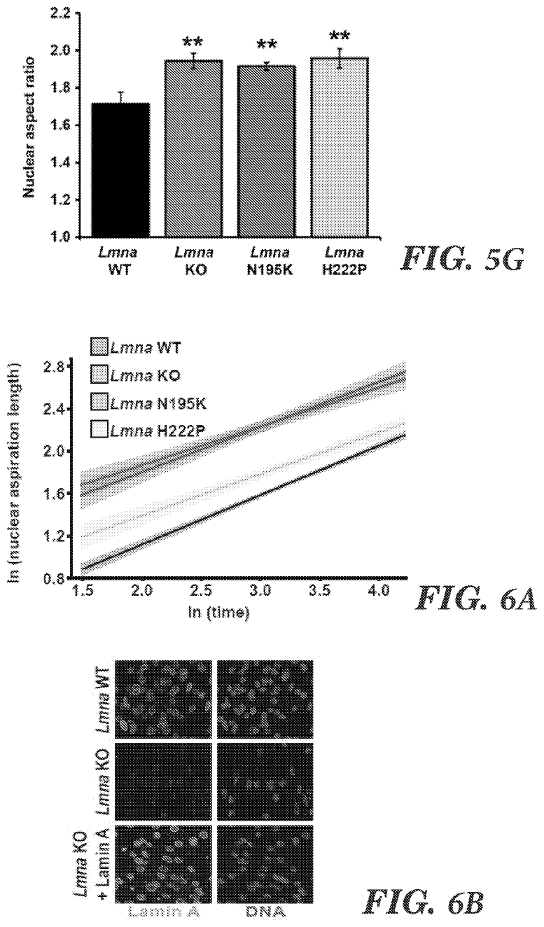

It was hypothesized that the progressive deterioration of Lmna mutant myofibers results from damage to mechanically weakened myonuclei exposed to cytoskeletal forces. To test this hypothesis, the nuclear deformability in primary myoblasts from the three laminopathy models were measured using a novel high throughput microfluidics-based micropipette aspiration assay (FIG. 5A) (Davidson et al., “High-Throughput Microfluidic Micropipette Aspiration Device to Probe Time-Scale Dependent Nuclear Mechanics in Intact Cells,” bioRxiv, 641084 (2019), which is hereby incorporated by reference in its entirety). Nuclei from Lmna KO and Lmna N195K myoblasts were substantially more deformable than nuclei from wild-type controls (FIGS. 5B-5C). Intriguingly, myoblasts from Lmna H222P mice, which have a later disease onset and less severe muscle defects than the other two Lmna mutant models (FIGS. 1A, 2A-2F), had only a modest increase in nuclear deformability relative to wild-type controls (FIGS. 5B-5C, 6A). Ectopic expression of lamin A significantly reduced the nuclear deformability defect in primary Lmna KO myoblasts (FIGS. 6B-6D), confirming that the impaired nuclear stability was a direct consequence of altering the nuclear lamina. In addition, primary myoblasts from Mdx mice, which develop mild muscular dystrophy due to loss of the cell membrane-associated protein dystrophin, had nuclear deformation indistinguishable from wild-type controls (FIG. 7), indicating that the defects in nuclear stability are specific to Lmna mutations and not muscular dystrophy in general.

To assess whether the observed defects in nuclear stability also occur in more mature, multinucleated myofibers, Lmna KO and wild-type myofibers were subjected to a ‘microharpoon’ assay, in which precise strain is exerted on the perinuclear cytoskeleton, and the induced nuclear deformation and displacement are used to infer nuclear stability and nucleo-cytoskeletal coupling, respectively (Fedorchak et al., “Cell Microharpooning to Study Nucleo-Cytoskeletal Coupling,” Methods Mol. Biol. 1411:241-254 (2016) and Lombardi et al.

“Biophysical Assays to Probe the Mechanical Properties of the Interphase Cell Nucleus: Substrate Strain Application and Microneedle Manipulation,” J. Vis. Exp. 55:3087 (2011), which are hereby incorproated by reference in their entirety). Lmna KO myofibers had significantly more deformable nuclei than wild-type controls (FIGS. 5D-5E), consistent with the micropipette aspiration results in the myoblasts. Furthermore, analysis of Lmna mutant and wild-type myofibers at day five of in vitro differentiation revealed that Lmna KO, Lmna N195K, and Lmna H222P myofibers had significantly elongated myonuclei compared to wild-type controls (FIGS. 5F-5G), consistent with decreased nuclear stability in the Lmna mutant cells and with previous reports of elongated nuclei in muscle biopsies from laminopathy patients (Tan et al., “Phenotype-Genotype Analysis of Chinese Patients with Early-Onset LMNA-Related Muscular Dystrophy,” PLoS One 10:e0129699 (2015), which is hereby incorporated by reference in its entirety). Taken together, these findings suggest that myopathic Lmna mutations that cause severe muscle defects result in mechanically weaker myonuclei.

### Example 3

### Lmna Mutant Myonuclei Display Chromatin Protrusions into the Cytoplasm

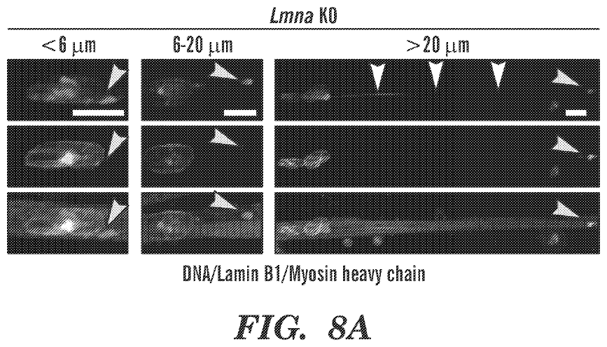

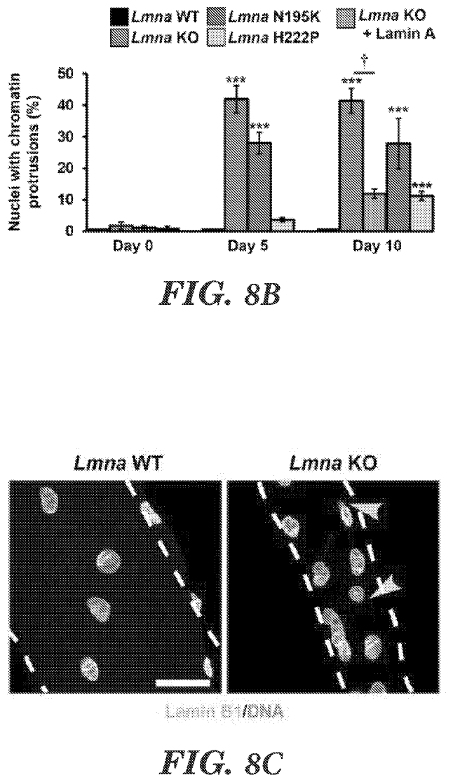

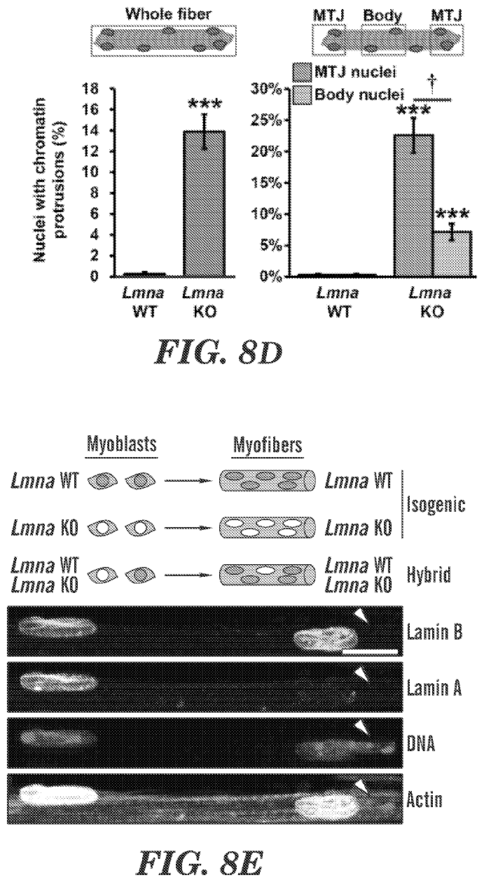

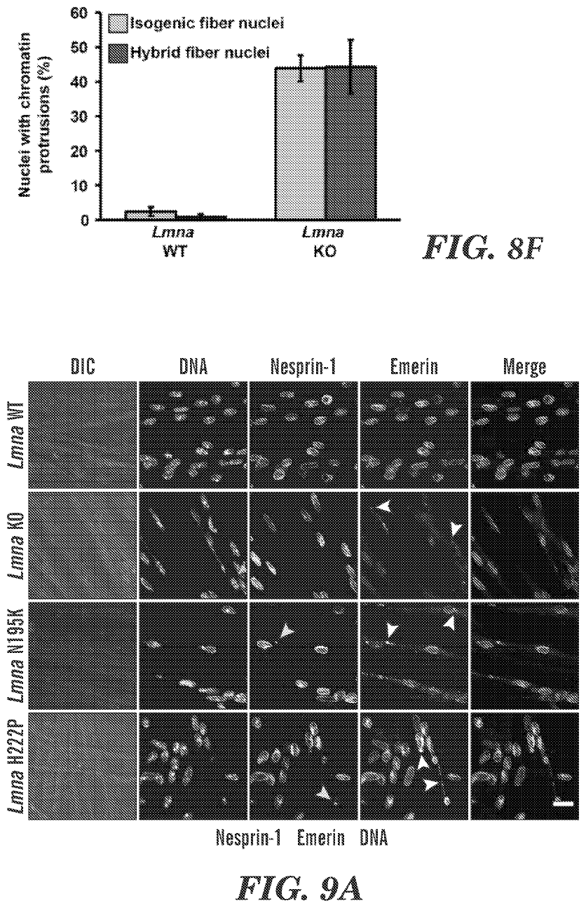

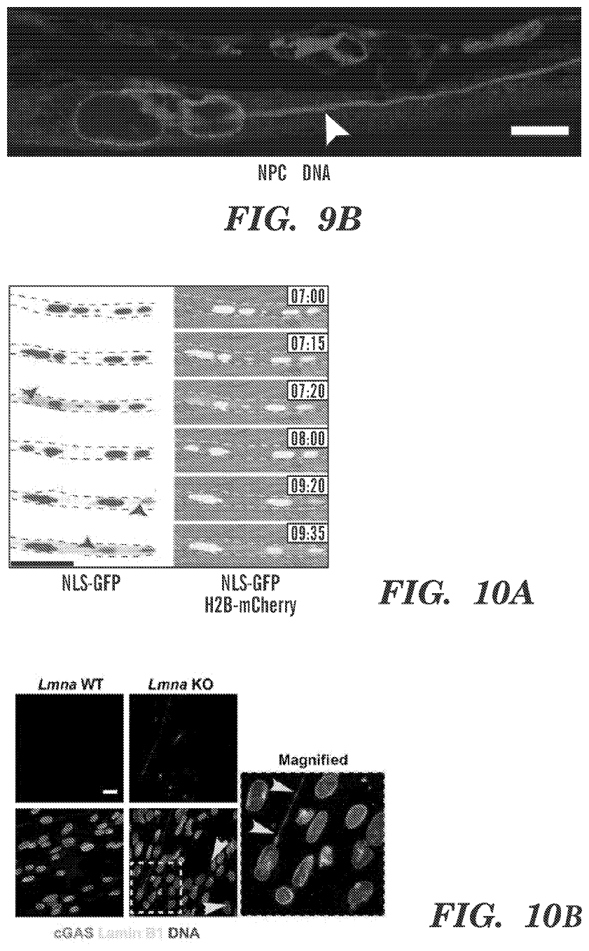

Physical stress on the nucleus during external compression or confined migration can induce chromatin protrusions across the nuclear lamina into the cytoplasm, particularly in lamin-deficient cells (Denais et al., “Nuclear Envelope Rupture and Repair During Cancer Cell Migration,” Science 352:353-358 (2016) and Le Berre et al., “Fine Control of Nuclear Confinement Identifies a Threshold Deformation Leading to Lamina Rupture and Induction of Specific Genes,” Integr. Biol. (Camb) 4(11):1406-1414 (2012), which are hereby incorporated by reference in their entirety). To test whether the mechanically weaker Lmna mutant myonuclei are prone to mechanically induced damage in muscle cells, nuclear structure and morphology were analyzed over the ten-day time course of differentiation and maturation of primary myoblasts from laminopathy mouse models and healthy controls. Despite their mechanically weaker nuclei (FIGS. 5A-5G), the Lmna mutant myoblasts show no nuclear abnormalities prior to differentiation (FIG. 8B). Following the onset of differentiation, however, Lmna KO, Lmna N195K, and Lmna H222P myofibers exhibited striking chromatin protrusions that were absent in wild-type fibers. These protrusions extended beyond the (B-type) nuclear lamina up to tens of microns into the cytoplasm (FIGS. 8A-8B). The protrusions were enclosed by nuclear membranes, as indicated by the frequent presence of the nuclear membrane protein emerin, and occasional presence of nesprin-1 (FIG. 9A); however, these nuclear envelope proteins were often concentrated in punctae inside the protrusions and myonuclei. Other nuclear envelope proteins, such as nuclear pore complex proteins, were largely absent from the protrusions (FIG. 9B), suggesting an altered membrane composition in the chromatin protrusion, similar to what has been reported in analogous structures in migrating cancer cells (Denais et al., “Nuclear Envelope Rupture and Repair During Cancer Cell Migration,” Science 352:353-358 (2016) and Maciejowski et al., “Chromothripsis and Kataegis Induced by Telomere Crisis,” Cell 163(7):1641-1654 (2015), which are hereby incorporated by reference in their entirety).

The frequency of chromatin protrusion was highest in Lmna KO myofibers, followed by Lmna N195K and then Lmna H222P myofibers (FIG. 8B), correlating with the increased nuclear deformability in vitro (FIGS. 5A-5G) and the disease severity in vivo (FIGS. 1A, 2A-2F). Intriguingly, while Lmna KO and Lmna N195K myofibers had extensive chromatin protrusions at day five of differentiation, the frequency of chromatin protrusions in the Lmna H222P cells was initially low, but increased from five to ten days of differentiation (FIG. 8B), matching the delayed disease onset and progressive phenotype of the Lmna H222P model in vivo. Ectopic expression of lamin A in Lmna KO myoblasts significantly reduced the occurrence of chromatin protrusions at ten days of differentiation (FIG. 8B), confirming that the protrusions were caused by loss of lamin A/C expression.

To confirm the results of the in vitro studies in vivo, single muscle fibers from the hindlimbs of Lmna KO and wild-type mice were isolated. Chromatin protrusions were not detectable in muscle fibers from wild-type mice (FIGS. 8C-8D). In contrast, nuclei in muscle fibers from Lmna KO mice had similar chromatin protrusions as observed in the in vitro differentiated myofibers (FIGS. 8C-8D). Interestingly, the prevalence of chromatin protrusions in the Lmna KO myonuclei strongly depended on the location within the muscle. Myonuclei at the myotendinous junction had a higher frequency of chromatin protrusions than nuclei in the muscle fiber body (FIG. 8D), consistent with a previous report of nuclear abnormalities at the myotendinous junction in Lmna KO mice (Gnocchi et al., “Uncoordinated Transcription and Compromised Muscle Function in the Lmna-Null Mouse Model of Emery- Emery-Dreyfuss Muscular Dystrophy,” PLoS One 6:e16651 (2011), which is hereby incorporated by reference in its entirety), and possibly reflecting increased mechanical stress at the myotendinous junction.

### Example 4

### Nuclear Damage is Intrinsic to Lamin A/C-Deficient Nuclei

To address whether the observed nuclear envelope defects in Lmna mutant muscle cells are nucleus-intrinsic or arise from altered signaling pathways or other cytoplasmic changes in the mutant cells, “hybrid” myofibers were generated by combining wild-type and Lmna KO myoblasts prior to differentiation. Following myoblast fusion, these cells formed multinucleated myotubes and myofibers that contained both wild-type and Lmna KO nuclei with a shared cytoplasm (FIG. 8E). Importantly, in skeletal muscle cells, each nucleus is thought to provide mRNA transcripts for the nearby cytoplasm (referred to as myonuclear domain), so that the local RNA and protein content primarily stems from the nearest myonucleus (Cutler et al., “Non-Equivalence of Nuclear Import Among Nuclei in Multinucleated Skeletal Muscle Cells,” J. Cell Sci. 131(3):jcs207670 (2018), which is herbey incorporated by reference in its entirety), and the genotype of each myonucleus can be determined by antibody staining against lamin A (FIG. 8E). The number of nuclei with chromatin protrusions was quantified and genetically identical nuclei (e.g., wild-type or Lmna KO) from hybrid and isogenic control myofibers after ten days of differentiation were compared (FIG. 8F). Hybrid myofibers comprising ˜80% wild-type nuclei and ˜20% Lmna KO nuclei appeared healthy. Nonetheless, Lmna KO nuclei within the hybrid myofibers showed the same relative frequency of chromatin protrusions as nuclei from isogenic Lmna KO myofibers (FIG. 8F). Conversely, wild-type nuclei in hybrid fibers lacked chromatin protrusions and were thus not adversely affected by the presence of Lmna KO nuclei in the shared cytoplasm (FIG. 8F). These results indicate that the defects in nuclear structure are intrinsic to the Lmna mutant myonuclei and not due to impaired muscle fiber health, altered cytoplasmic signaling, or changes in the cytoplasmic architecture in Lmna mutant muscle fibers.

### Example 5

### Lmna Mutant Myofibers Exhibit Extensive Nuclear Envelope Rupture in vitro

Physical compression by cytoskeletal forces can result in nuclear envelope rupture, with depletion of lamins exacerbating the frequency of nuclear envelope rupture (Denais et al., “Nuclear Envelope Rupture and Repair During Cancer Cell Migration,” Science 352:353-358 (2016); Raab et al., “ESCRT III Repairs Nuclear Envelope Ruptures During Cell Migration to Limit DNA Damage and Cell Death,” Science 352(6283):359-362 (2016); Robijns et al., “In Silico Synchronization Reveals Regulators of Nuclear Ruptures in Lamin A/C Deficient Model Cells,” Sci. Rep. 6:30325 (2016); De Vos et al., “Repetitive Disruptions of the Nuclear Envelope Invoke Temporary Loss of Cellular Compartmentalization in Laminopathies,” Hum. Mol. Genet. 20:4175-4186 (2011); Hatch et al., “Nuclear Envelope Rupture is Induced by Actin-Based Nucleus Confinement,” J. Cell Biol. 215:27-36 (2016); and Vargas et al., “Transient Nuclear Envelope Rupturing During Interphase in Human Cancer Cells,” Nucleus 3:88-100 (2012), which are hereby incorporated by reference in their entirety). To examine whether the reduced nuclear stability seen in Lmna mutant muscle cells (FIGS. 5A-5G) leads to nuclear envelope rupture in Lmna mutant myofibers, primary myoblasts were modified to co-express a fluorescent nuclear envelope rupture reporter, consisting of a green fluorescent protein with a nuclear localization signal (NLS-GFP) (Denais et al., “Nuclear Envelope Rupture and Repair During Cancer Cell Migration,” Science 352:353-358 (2016), which is hereby incorporated by reference in its entirety) and fluorescently labeled histone (H2B-tdTomato). NLS-GFP is normally localized to the nucleus, but rapidly spills into the cytoplasm upon loss of nuclear membrane integrity and is then gradually reimported into the nucleus after the nuclear membrane has been repaired (Denais et al., “Nuclear Envelope Rupture and Repair During Cancer Cell Migration,” Science 352:353-358 (2016), which is hereby incorporated by reference in its entirety). In vitro differentiated Lmna KO myotubes frequently exhibited nuclear envelope ruptures (FIG. 10A), which were absent in wild-type controls. To investigate nuclear envelope rupture in more detail, primary myoblasts were stably modified with another fluorescent nuclear envelope rupture reporter, cGAS-mCherry, which accumulates at the rupture site (Denais et al., “Nuclear Envelope Rupture and Repair During Cancer Cell Migration,” Science 352:353-358 (2016), which is hereby incorporated by reference in its entirety) (FIG. 10B). Unlike the transient cytoplasmic NLS-GFP signal, however, the cGAS-mCherry accumulation persists even after the nuclear envelope has been repaired (Denais et al., “Nuclear Envelope Rupture and Repair During Cancer Cell Migration,” Science 352:353-358 (2016) and Raab et al., “ESCRT III Repairs Nuclear Envelope Ruptures During Cell Migration to Limit DNA Damage and Cell Death,” Science 352(6283):359-362 (2016), which are hereby incorporated by reference in their entirety). Wild-type myotubes had no detectable accumulation of cGAS-mCherry (FIG. 10C). In contrast, Lmna KO myotubes displayed a progressive increase in the number of nuclear cGAS-mCherry foci during differentiation, starting around day two, which could be rescued by ectopic expression of wild-type lamin A (FIG. 10C). LmnaN195K showed intermediate levels of nuclear envelope rupture (FIG. 11A), whereas Lmna H222P myotubes had cGAS-mCherry accumulation comparable to wild-type controls (FIG. 11B), consistent with the milder defects in nuclear stability in the Lmna H222P mutant cells (FIGS. 5B-5C, 6A).

### Example 6

### Lamin A/C-Deficient Myofibers Experience Extensive Nuclear Envelope Rupture In Vivo

To test whether nuclear envelope rupture occurs within Lmna KO muscle in vivo, transgenic mice that express a fluorescent cGAS-tdTomato nuclear rupture reporter were generated and crossed with the Lmna mutant mouse models. Single hindlimb muscle fibers isolated from Lmna KO offspring expressing the cGAS-tdTomato reporter revealed a large fraction of myonuclei with cGAS-tdTomato foci, which were absent in both wild-type littermates expressing the cGAS-tdTomato reporter and in Lmna KO mice that were negative for the cGAS-tdTomato reporter (FIGS. 10D-10F). Within Lmna KO muscle fibers, the frequency of nuclear envelope rupture was significantly higher at the myotendinous junction than in the myofiber body nuclei (FIGS. 10E, 12), consistent with the increased frequency of chromatin protrusions in the myotendinous junction myonuclei. The amount of cGAS-tdTomato accumulation scaled with disease severity: Lmna KO fibers had the highest amount of cGAS-tdTomato foci, Lmna N195K fibers had an intermediate amount, and Lmna H222P fibers had no nuclear cGAS-tdTomato accumulation, closely matching the in vitro data (FIG. 11C). As an independent approach to detect loss of nuclear-cytoplasmic compartmentalization in muscle fibers, the intracellular localization of endogenous heat shock protein 90 (Hsp90), which is typically excluded from the nucleus in healthy muscle fibers, was analyzed (Park et al., “DNA-PK Promotes the Mitochondrial, Metabolic, and Physical Decline that Occurs During Aging,” Cell Metab. 25(5):1135-1146 (2017), which is hereby incorporated by reference in its entirety). In in vitro assays, Hsp90 was cytoplasmic in wild-type myofibers, whereas all three Lmna mutant models had increased nuclear Hsp90 levels during myoblast differentiation (FIGS. 13A-13B). Similarly, muscle fibers isolated from Lmna KO mice, but not wild-type littermates, had a significant increase in nuclear Hsp90 (FIGS. 13C-13D), confirming the occurrence of nuclear envelope rupture in vivo. Taken together, these findings indicate widespread nuclear envelope rupture in severe cases of laminopathic skeletal muscle.

### Example 7

### Lmna KO Myonuclei have Increased Levels of DNA Damage In Vitro and In Vivo

Recent studies found that nuclear deformation and nuclear envelope rupture can cause DNA damage in migrating cells (Denais et al., “Nuclear Envelope Rupture and Repair During Cancer Cell Migration,” Science 352:353-358 (2016); Raab et al., “ESCRT III Repairs Nuclear Envelope Ruptures During Cell Migration to Limit DNA Damage and Cell Death,” Science 352(6283):359-362 (2016); and Irianto et al., “Nuclear Constriction Segregates Mobile Nuclear Proteins away from Chromatin,” Mol. Biol. Cell. 27(25):4011-4020 (2016), which are hereby incorporated by reference in their entirety). To investigate whether chromatin protrusions and nuclear envelope rupture can similarly lead to DNA damage in muscle cells, DNA damage was quantified in differentiating primary myoblasts by staining for γH2AX, a marker for double stranded DNA damage (Blackford et al., “ATM, ATR, and DNA-PK: The Trinity at the Heart of the DNA Damage Response,” Mol. Cell 66(6):801-817 (2017), which is hereby incorporated by reference in its entirety). Both Lmna KO and wild-type myoblasts had elevated levels of DNA damage at the onset of differentiation (FIGS. 14A-14B), consistent with previous reports that show the transition from myoblasts to myotubes is associated with a transient increase in γH2AX levels (Larsen et al., “Caspase 3/Caspase-Activated DNase Promote Cell Differentiation by Inducing DNA Strand Breaks,” PNAS USA 107:4230-4235 (2010) and Connolly et al., “DNA-PK Activity is Associated with Caspase-Dependent Myogenic Differentiation,” The FEBS Journal 283(19):3626-3636 (2016), which are hereby incorproated by reference in their entirety). However, while γH2AX levels in wild-type myotubes subsequently decreased and then remained stable at low levels, the fraction of myonuclei with severe DNA damage in the Lmna KO cells continued to increase from five to ten days post-differentiation, with nearly 20% of Lmna KO myonuclei exhibiting severe DNA damage at day ten (FIG. 14B). Consistent with the increased DNA damage, Lmna KO myotubes exhibited significantly increased activity of the DNA-dependent protein kinase, DNA-PK (FIG. 14C), one of the major DNA damage response pathways in post-mitotic cells (Blackford et al., “ATM, ATR, and DNA-PK: The Trinity at the Heart of the DNA Damage Response,” Mol. Cell 66(6):801-817 (2017), which is hereby incorporated by reference in its entirety). Single muscle fibers isolated from Lmna KO mice similarly contained many myonuclei with extensive γH2AX staining (FIGS. 14D-14E) and increased DNA-PK activity, especially at the myotendinous junction, where over 10% of nuclei show very high intensity staining (FIGS. 14F, 15), confirming the presence of extensive DNA damage in Lmna KO muscle fibers in vivo. The Lmna N195K single muscle fibers showed similar high levels of DNA damage compared to Lmna KO mice, whereas the Lmna H222P single muscle fibers showed an increase only in mid and low levels of DNA damage, consistent with the milder disease severity and other phenotypic markers (FIG. 14E). In contrast, muscle fibers isolated from wild-type mice contained only low levels of DNA damage (FIGS. 14D-14E). Of the myonuclei with extensive γH2AX staining, 82% also had chromatin protrusions, suggesting a link between physical damage to the nucleus and DNA damage (FIG. 16).

To determine whether the accumulation of DNA damage during in vitro differentiation of Lmna KO myoblasts was caused by progressive new nuclear damage or defects in DNA damage repair, which had been reported in several progeroid laminopathies (Burla et al., “Genomic Instability and DNA Replication Defects in Progeroid Syndromes,” Nucleus 9:368-379 (2018); Larrieu et al., “Chemical Inhibition of NAT 10 Corrects Defects of Laminopathic Cells,” Science 344:527-532 (2014); and Hutchison et al., “The Role of DNA Damage in Laminopathy Progeroid Syndromes,” Biochem. Soc. Trans. 39:1715-1718 (2011), which are hereyb incorporated by reference in their entirety), Lmna KO and wild-type myofibers were subjected to a pulse of gamma irradiation and monitored γH2AX levels at 3, 6, and 24 hours post-treatment. Consistent with previous studies (Polo et al., “Dynamics of DNA Damage Response Proteins at DNA Breaks: A Focus on Protein Modifications,” Genes Dev. 25:409-433 (2011), which is hereby incorporated by reference in its entirety), irradiation resulted in a rapid increase in the number of γH2AX foci at 3 hours that then gradually resolved and returned to baseline by 24 hours post irradiation (FIGS. 17A-17B). Notably, following irradiation, Lmna KO myofibers displayed a DNA damage profile nearly identical to wild-type controls, suggesting that their ability to repair DNA damage is not significantly impaired, and the accumulation of DNA damage in the myotubes is more likely due to new incidents of damage.

### Example 8

### Accumulation of DNA Damage Correlates with Myofiber Death

To test whether accumulation of DNA damage is sufficient to explain the progressive decline in myofiber viability in the Lmna KO cells, Lmna KO and wild-type myofibers were subjected to repeated treatments of phleomycin, a radiation mimetic agent, in conjuction with inhibition of DNA damage repair with NU7441, a DNA-PK-specific inhibitor, and/or KU55933, an ATM-specific inhibitor (FIG. 14G). Combined treatment with phleomycin and DNA damage repair inhibition (by one or both inhibitors in combination) resulted in an accumulation of DNA damage and loss of viability in wild-type myofibers comparable to that observed in untreated Lmna KO cells (FIGS. 14H-14I, 18). In contrast, phleomycin, alone or in combination with DNA damage repair inhibition, did not further reduce viability in Lmna KO myofibers (FIG. 18), suggesting that the preexisting DNA damage in these cells is already sufficient to drive myofiber decline. Since different types of DNA damage can elicit distinct repair responses in post-mitotic muscle cells (Fortini et al., “DNA Damage Response by Single-Strand Breaks in Terminally Differentiated Muscle Cells and the Control of Muscle Integrity,” Cell Death Diff 19(11):1741-1749 (2012), which is herbey incorporated by reference in its entirety), the DNA damage and ensuing repair response associated with nuclear envelope rupture may differ from that induced by either phleomycin or irradiation. Thus, it cannot be ruled out that the Lmna KO cells may exhibit some defects in DNA damage repair, or that additional mechanisms contribute to the pathogenesis in the Lmna mutant cells.

### Example 9

### Nuclear Damage in Lmna KO Myofibers can be Prevented by Microtubule Stabilization

It was surmised that nuclear envelope ruptures in Lmna mutant myofibers resulted from cytoskeletal forces acting on mechanically weak myonuclei, and that reducing mechanical stress on the nuclei would decrease nuclear damage. In striated muscle cells, the microtubule network remodels significantly upon differentiation to form a cage-like structure around the myonuclei (Gimpel et al., “Nesprin- 1α-Dependent Microtubule Nucleation from the Nuclear Envelope via Akap450 is Necessary for Nuclear Positioning in Muscle Cells,” Curr. Biol. 27(19):2999-3009 (2017), which is hereby incorporated by reference in its entirety)(FIG. 19), in large part due to the redistribution of centrosomal proteins at the nuclear envelope (Starr et al., “Muscle Development: Nucleating Microtubules at the Nuclear Envelope,” Curr. Biol. 27(19):R1071-R1073 (2017), which is hereby incorporated by reference in its entirety). To test if stabilizing this microtubule network and thereby reinforcing myonuclei can reduce chromatin protrusions and nuclear envelope rupture, in vitro differentiated myoblasts were treated with low doses of the microtubule stabilizing drug, paclitaxel. Here, the focus was on the Lmna KO model, which showed the most severe nuclear defects. The microharpoon assay confirmed that microtubule stabilization reinforced Lmna KO nuclei in differentiated myofibers and significantly reduced nuclear deformation in response to cytoplasmic force application (FIGS. 20A-20B). Furthermore, paclitaxel treatment significantly reduced the percentage of nuclei with chromatin protrusions (FIG. 20C) and the incidence of nuclear envelope rupture detected with the cGAS-mCherry reporter in the Lmna KO cells (FIG. 20D), suggesting that nuclear damage indeed arises from mechanical stress on the myonuclei and can be prevented by mechanically stabilizing the nuclei.

### Example 10

### Kinesin-Mediated Nuclear Movements are Responsible for Nuclear Damage in Lmna KO Myonuclei

The findings that nuclear damage occurred only during myoblast differentiation, when the cytoskeleton significantly remodels, and that microtubule stabilization significantly reduced the amount of nuclear damage in Lmna KO myofibers, suggest that the nuclear defects result from cytoskeletal forces acting on the mechanically weaker Lmna mutant myonuclei. Physical stress may be imparted to myonuclei via (1) actomyosin-mediated contractile forces, and/or (2) forces due to nuclear movements at various stages of muscle development, including myoblast migration and fusion (Chang et al., “Accessorizing and Anchoring the LINC Complex for Multifunctionality,” J. Cell Biol. 208(1):11-22 (2015), which is hereby incorporated by reference in its entirety), microtubule-driven spacing (Gimpel et al., “Nesprin-1α-Dependent Microtubule Nucleation from the Nuclear Envelope via Akap450 is Necessary for Nuclear Positioning in Muscle Cells,” Curr. Biol. 27(19):2999-3009 (2017); Elhanany-Tamir et al., “Organelle Positioning in Muscles Requires Cooperation Between two KASH Proteins and Microtubules,” J. Cell Biol. 198:833-846 (2012); Espigat-Georger et al., “Nuclear Alignment in Myotubes Requires Centrosome Proteins Recruited by Nesprin-1,” J. Cell Sci. 129:4227-4237 (2016); Folker et al., “Translocating Myonuclei have Distinct Leading and Lagging Edges that Require Kinesin and Dynein,” Development 141:355-366 (2014); Meinke et al., “Muscular Dystrophy-Associated SUN1 and SUN2 Variants Disrupt Nuclear-Cytoskeletal Connections and Myonuclear Organization,” PLoS Genet. 10:e1004605 (2014); Metzger et al., “MAP and Kinesin-Dependent Nuclear Positioning is Required for Skeletal Muscle Function,” Nature 484:120-124 (2012); Stroud et al., “Nesprin la2 is Essential for Mouse Postnatal Viability and Nuclear Positioning in Skeletal Muscle,” J. Cell Biol. jcb. 201612128 (2017); Wilson et al., “Opposing Microtubule Motors Drive Robust Nuclear Dynamics in Developing Muscle Cells,” J. Cell Sci. 125:4158-4169 (2012); and Wilson et al., “Nesprins Anchor Kinesin-1 Motors to the Nucleus to Drive Nuclear Distribution in Muscle Cells,” Development 142:218-228 (2015), which are hereby incorporated by reference in their entirety), shuttling to the periphery of myofibers (Falcone et al., “N-WASP is Required for Amphiphysin-2/BIN1-Dependent Nuclear Positioning and Triad Organization in Skeletal Muscle and is Involved in the Pathophysiology of Centronuclear Myopathy,” EMBO Molecular Medicine 6:1455-1475 (2014); Roman et al., “Myofibril Contraction and Crosslinking Drive Nuclear Movement to the Periphery of Skeletal Muscle,” Nat. Cell Biol. 19:1189-1201 (2017); and D′Alessandro et al., “Amphiphysin 2 Orchestrates Nucleus Positioning and Shape by Linking the Nuclear Envelope to the Actin and Microtubule Cytoskeleton,” Developmental Cell 35:186-198 (2015), which are hereby incorporated by reference in their entirety), and anchoring at the fiber periphery and neuromuscular junctions (Bone et al. “Nuclear Migration Events Throughout Development,” J. Cell Sci. 129:1951-1961 (2016), which is hereby incorporated by reference in its entirety). Recently, actomyosin contraction was shown to be a primary driver of nuclear envelope rupture in chick cardiac tissue, and this nuclear envelope rupture was rescued by treatment with blebbistatin (Cho et al., “Mechanosensing by the Lamina Protects Against Nuclear Rupture, DNA Damage, and Cell-Cycle Arrest,” Dev. Cell 49:920-935 e925 (2019), which is hereby incorporated by reference in its entirety). To address whether nuclear envelope damage in myotubes was caused by actomyosin contractility, Lmna KO and wild-type myotubes were treated with nifedipine, a muscle-specific calcium channel blocker. Nifedipine treatment effectively abrogated myotube contraction, but did not reduce the frequency of chromatin protrusions and nuclear envelope ruptures in Lmna KO myonuclei (FIGS. 21A-21B), indicating that actomyosin contractility is not required to induce nuclear envelope damage. The differences in these results may stem from differences in contraction strength between in vitro and in vivo systems as well as from the differences between cardiac and skeletal muscle cells. Therefore, attention was focused to cytoskeletal forces exerted on the nucleus during nuclear migration in differentiating myotubes.

Based on the timing of the onset of nuclear envelope rupture (FIG. 10C) and a progressive increase in the length of the chromatin tethers with differentiation (FIG. 22), the role of microtubule driven myonuclear spreading during myofiber maturation was examined (Wilson et al., Nesprins Anchor Kinesin-1 Motors to the Nucleus to Drive Nuclear Distribution in Muscle Cells,” elopment 142(1):218-228 (2015) and Roman et al., “Nuclear Positioning in Skeletal Muscle,” Sem. Cell Dev. Biol. 82:51-56 (2018), which are hereby incorporated by reference in their entirety). Time-lapse sequences of Lmna KO myoblasts expressing the NLS-GFP and/or cGAS-mCherry reporters revealed that nuclear envelope rupture frequently occurred when myonuclei were moved along the length of myotubes by microtubule-associated motors (FIG. 23A). Thus, it was reasoned that inhibiting nuclear movement should prevent nuclear damage in Lmna KO myofibers. Supporting this hypothesis, depletion of Kif5b (FIG. 24A), a subunit of kinesin-1 that is required for nuclear migration in myotubes (Wilson et al., Nesprins Anchor Kinesin-1 Motors to the Nucleus to Drive Nuclear Distribution in Muscle Cells,” Development 142(1):218-228 (2015); Roman et al., “Nuclear Positioning in Skeletal Muscle,” Sem. Cell Dev. Biol. 82:51-56 (2018); and Gache et al., Microtubule Motors Involved in Nuclear Movement During Skeletal Muscle Differentiation,” Mol. Biol. Cell 28:865-874 (2017), which are hereby incorporated by reference in their entirety), nearly abolished chromatin protrusions (FIG. 24B), nuclear envelope rupture (FIGS. 23B-23C), and high levels of DNA damage in the Lmna KO myotubes (FIGS. 24C-24D). These findings indicate that nuclear movement by microtubule-mediated forces are sufficient to cause nuclear damage in Lmna mutant myofibers that correlates with increased DNA damage in these cells.

### Example 11

### Reducing the Mechanical Forces Acting on Lmna KO Myonuclei Improve Myofiber Function and Viability

If mechanically induced nuclear envelope rupture and DNA damage are causative for the decline in viability and contractility in the Lmna KO myofibers, then reducing the cytoskeletal forces on the myonuclei should improve myofiber health. Since depletion of Kif5b has detrimental long-term effects on myofiber function (Starr, D. A., “Muscle Development: Nucleating Microtubules at the Nuclear Envelope,” Curr. Biol. 27(19):R1071-R1073 (2017), which is hereby incorporated by reference in its entirety), a system where the Linker of Nucleoskeleton and Cytoskeleton (LINC) is disrupted through expression of a dominate negative GFP-KASH2 (DN-KASH) protein (Stewart-Hutchinson et al., “Structural Requirements for the Assembly of LINC Complexes and their Function in Cellular Mechanical Stiffness,” Exp. Cell Res. 314:1892-1905 (2008), which is hereby incorporated by reference in its entirety) was used (FIG. 24A). As a control, a similar construct that contained a double alanine extension was generated (DN-KASHext). The DN-KASHext construct still targets to the NE, but cannot disrupt the LINC complex (Stewart-Hutchinson et al., “Structural Requirements for the Assembly of LINC Complexes and their Function in Cellular Mechanical Stiffness,” Exp. Cell Res. 314:1892-1905 (2008), which is hereby incorporated by reference in its entirety), as confirmed by the retention of nesprin-1 at the nuclear envelope (FIG. 25A). The DN-KASH constructs were expressed under the control of an inducible promotor that allowed for controlling the onset of LINC complex disruption following the initial fusion events of myogenesis (FIG. 25B). This experimental approach reduced force transmission from the cytoskeleton to the nucleus and limited nuclear spreading in the DN-KASH cells, but not the DN-KASHext cells, similar to the depletion of Kif5b (FIG. 25B). Expression of the DN-KASH constructs had no effect on myofiber contractility or viability in Lmna WT myofibers (FIGS. 25C-25D). Expression of the DN-KASH construct, but not the DN-KASHext, starting on day three of differentiation significantly reduced the number of chromatin protrusions and incidence of nuclear envelope rupture in Lmna KO cells (FIGS. 23D-12E, 25E). Importantly, this reduction in nuclear damage was accompanied by a reduction in the fraction of cells with severe DNA damage (FIGS. 25F) and substantially improved myofiber contractility and viability (FIGS. 23F-23H). These findings further support the model that mechanical forces induce nuclear envelope rupture and DNA damage in Lmna mutant myofibers, leading to myofiber dysfunction and death.

### Example 12

### Skeletal Muscle Biopsies from Patients with LMNA-Associated Muscular Dystrophy show DNA Damage

Finally, to corroborate the findings from the in vitro and in vivo mouse models of striated muscle laminopathies in a clinically relevant context, skeletal muscle biopsy samples from humans with LA/INA-related muscular dystrophies and age-matched controls were examined (Table 1). Muscle cryosections were immunofluorescently labeled for 53BP1, an established marker for DNA double strand breaks (Bekker-Jensen et al., “Dynamic Assembly and Sustained Retention of 53BP1 at the Sites of DNA Damage are Controlled by Mdcl/NFBD1,” J. Cell Biol. 170(2):201-211 (2005) and Loewer et al., “The p53 Response in Single Cells is Linearly Correlated to the Number of DNA Breaks Without a Distinct Threshold,” BMC Biology 11:114 (2013), which are hereby incorporated by reference in their entirety), and myofiber nuclei were identified based on labeling for DNA, actin, and dystrophin (FIGS. 26A-26B, 27A-27B). Intriguingly, tissues from individuals with the most severe forms of the muscular dystrophy, i.e., those with early childhood and juvenile onsets, had significantly increased DNA damage compared to age-matched controls (FIGS. 26C-26E), thus closely mirroring the findings in the most severe laminopathy mouse models (FIG. 14E).

TABLE 1
Description of LMNA Patients and Muscle Biopsy Samples used for Immunofluorescence Analysis.

| | | | | Age of | Age when muscle | | |
|---|---|---|---|---|---|---|---|
| Nucleotide | Amino acid | Age on | | diagnosis | biopsy obtained | | |
| change | substitution | onset | | (years) | (years) | Tentative diagnosis/comments | Reference |
| 745C > T | R249W | 3 | months | <1 | 2 | Congenital MD, early onset with | * |
| | | | | | | dropped head, unable to sit | |
| | | | | | | unassisted at age 3 years | |
| 1346G > T | G449V | 6 | months | 3 | 1 | Congenital MD, non-ambulatory at | ** |
| | | | | | | age 13 years | |
| 1466T > C | L489P | <2 | | 7 | 2 | Congenital MD , moderately severe | ** |
| | | | | | | at age 2 years | |
| 1540T > C | W514R | 2-3 | years | 5 | 3 | Mild to moderate myopathy at age | ** |
| | | | | | | 14 years | |

| | | | | | | |
|---|---|---|---|---|---|---|
| 1357C > T | R453W | n.a. | 6 | 10 | Mild myopathy at age 5.5 years | * |
| 1622G > C | R541P | n.a. | <52 | 52 | n.a. | * |

n.a.; not available
* unpublished
** Dialynas et al., “LMNA Variants Cause Cytoplasmic Distribution of Nuclear Pore Proteins in Drosophila and Human Muscle,” Hum. Mol. Genet. 21: 1544-1556 (2012) and Dialynas et al., “Myopathic Lamin Mutations Cause Reductive Stress and Activate the nrf2/keap-1 Pathway,” PLoS Genet 11:e1005231 (2015), which are hereby incorporated by reference in their entirety.

### Discussion of Examples 1-12

The mechanisms by which LMNA mutations result in skeletal muscle-specific diseases, such as AD-EDMD and LMNA-CMD, has long puzzled researchers and clinicians, presenting a major hurdle in the development of effective treatment approaches. The Examples described herein provide comprehensive new evidence in support of the ‘mechanical stress’ hypothesis. By using three mouse models of striated muscle laminopathies with varying onset and severity, the frequency of nuclear defects, including chromatin protrusions, nuclear envelope rupture, and DNA damage, was systematically assessed in muscle fibers in vitro and in vivo, in a uniform genetic background. These studies revealed a striking correlation between the nuclear defects observed during in vitro myoblast differentiation, myonuclear defects in isolated muscle fibers, and disease onset and severity in the mouse model. Notably, analysis of muscle biopsy tissue from individuals with LMNA muscular dystrophies showed a similar trend, with samples corresponding to the most severe forms of muscular dystrophy having the largest fraction of myonuclei with DNA damage. The paucity of available human muscle samples and effects of genetic background in these diseases, limit the conclusions that can be made.

The findings of nuclear envelope rupture are consistent with reports of nuclear envelope damage and intrusion of cytoplasmic organelles into the nucleoplasm in skeletal muscle fibers of patients with EDMD (Vigouroux et al., “Nuclear Envelope Disorganization in Fibroblasts from Lipodystrophic Patients with Heterozygous R482Q/W Mutations in the Lamin A/C Gene,” J. Cell. Sci. 114:4459-4468 (2001); Fidzianska et al., “Architectural Abnormalities in Muscle Nuclei. Ultrastructural Differences Between X-Linked and Autosomal Dominant Forms of EDMD,” J. Neurol. Sci. 210:47-51 (2003); Fidzianska et al., “Ultrastructural Abnormality of Sarcolemmal Nuclei in Emery-Dreifuss Muscular Dystrophy (EDMD),” J. Neurol. Sci. 159:88-93 (1998); and Park et al., “Nuclear Changes in Skeletal Muscle Extend to Satellite Cells in Autosomal Dominant Emery-Dreifuss Muscular Dystrophy/Limb-Girdle Muscular Dystrophy 1B,” Neuromuscul. Disord. 19(1):29-36 (2009), which are hereby incorporated by reference in their entirety), cardiac myocytes in LMNA-dilated cardiomyopathy patients (Gupta et al., “Genetic and Ultrastructural Studies in Dilated Cardiomyopathy Patients: A Large Deletion in the Lamin A/C Gene is Associated with Cardiomyocyte Nuclear Envelope Disruption,” Basic Research in Cardiology 105:365-377 (2010) and Sylvius et al., “In Vivo and In Vitro Examination of the Functional Significances of Novel Lamin Gene Mutations in Heart Failure Patients,” J. Med. Genet. 42:639-647 (2005), which is hereby incorporated by reference in its entirety), lamin A/C-deficient mice (Sullivan et al., “Loss of A-type Lamin Expression Compromises Nuclear Envelope Integrity Leading to Muscular Dystrophy,” J. Cell Biol. 147:913-920 (1999) and (Nikolova et al., “Defects in Nuclear Structure and function Promote Dilated Cardiomyopathy in Lamin A/C-Deficient Mice,” The Journal of Clinical Investigation 113:357-369 (2004), which are hereby incorporated by reference in their entirety), and muscle and tendons of lamin-deficient fruit flies (Uchino et al., “Loss of Drosophila A-Type Lamin C Initially Causes Tendon Abnormality Including Disintegration of Cytoskeleton and Nuclear Lamina in Muscular Defects,” Dev. Biol. 373:216-227 (2013) and Dialynas et al., “LMNA Variants Cause Cytoplasmic Distribution of Nuclear Pore Proteins in Drosophila and Human Muscle,” Hum. Mol. Genet. 21:1544-1556 (2012), which are hereby incorporated by reference in their entirety). Unlike those previous reports, however, the results presented herein provide detailed information on the extent, timing, and cause of the nuclear envelope damage, revealing a striking correlation of nuclear envelope rupture and disease severity.

Although increased DNA damage and altered DNA damage repair have been reported previously in LMNA mutant cells, those cases were linked to progeroid diseases, including Hutchinson-Gilford progeria syndrome (HGPS) and atypical Werner syndrome (AWS) (Burla et al., “Genomic Instability and DNA Replication Defects in Progeroid Syndromes,” Nucleus 9:368-379 (2018); Larrieu et al., “Chemical Inhibition of NATIO Corrects Defects of Laminopathic Cells,” Science 344:527-532 (2014); and Hutchison et al., “The Role of DNA Damage in Laminopathy Progeroid Syndromes,” Biochem. Soc. Trans. 39:1715-1718 (2011); Graziano et al., “Causes and Consequences of Genomic Instability in Laminopathies: Replication Stress and Interferon Response,” Nucleus 9:258-275 (2018); and Chen et al., “DNA Damage Response/TP53 Pathway is Activated and Contributes to the Pathogenesis of Dilated Cardiomyopathy Associated with LMNA (Lamin A/C) Mutations,” Circ. Res. 124:856-873 (2019), which are hereby incorporated by reference in their entirety). The findings presented herein demonstrate for the first time that cytoskeletal forces acting on myonuclei result in nuclear damage and DNA damage in skeletal muscle fibers in vitro and in vivo. The precise mechanisms by which nuclear envelope damage and nuclear envelope rupture could cause DNA damage and cell death remains to be elucidated. The DNA damage could arise from exposure of genomic DNA to cytoplasmic nucleases following nuclear envelope rupture, or nuclear exclusion and efflux of DNA repair factors, as previously discussed in the context of confined cell migration (Hatch, E. M., “Nuclear Envelope Rupture: Little Holes, Big Openings,” Curr. Opin. Cell Biol. 52:66-72 (2018); Shah et al., “Bursting the Bubble—Nuclear Envelope Rupture as a Path to Genomic Instability?,” Trends in Cell Biology 27:546-555 (2017); and Irianto et al., “DNA Damage Follows Repair Factor Depletion and Portends Genome Variation in Cancer Cells after Pore Migration,” Curr. Biol. 27(2):210-223 (2017), which are hereby incorporated by reference in their entirety). The correlation between DNA damage and nuclear envelope defects in the studies presented herein (FIG. 16) suggests that the DNA damage is linked to mechanically induced nuclear envelope defects, which is further supported by the finding that depletion of kinesin-1 and disruption of the LINC complex abolished nuclear envelope defects and the most severe DNA damage in Lmna KO cells (FIGS. 23B-23E, 24B-24D, 25E-25F). Given the known association between lamin A/C and the DNA damage response protein 53BP1 (Mayca Pozo et al., “Regulatory Cross-Talk Determines the Cellular Levels of 53BP1 Protein, a Critical Factor in DNA Repair,” Journal of Biological Chemistry 292:5992-6003 (2017), which is hereby incorporated by reference in its entirety), loss of lamin A/C could also impair DNA damage repair efficiency, although this effect may be limited to proliferating cells and the associated replication stress (Gibbs-Seymour et al., “Lamin A/C-Dependent Interaction with 53BP1 Promotes Cellular Responses to DNA Damage,” Aging Cell 14:162-169 (2015); Singh et al., “Lamin A/C Depletion Enhances DNA Damage-Induced Stalled Replication Fork Arrest,” Mol. Cell Biol. 33:1210-1222 (2013); Redwood et al., “A Dual Role for A-Type Lamins in DNA Double-Strand Break Repair,” Cell Cycle 10:2549-2560 (2011), which are hereby incorporated by reference in their entirety). In the experiments presented herein, post-mitotic Lmna KO myofibers exposed to DNA-damaging irradiation exhibited similar DNA damage repair dynamics as wild-type cells, indicating that the observed increase in DNA damage in Lmna KO myofibers is not caused by defective DNA damage repair. Nonetheless, it is possible that the efficiency of DNA damage repair decreases over the course of the differentiation, as the highest levels of DNA damage were found in myofibers at late stages of maturation. Such a decrease in DNA repair efficiency is consistent with work showing that satellite cells repair radiation-induced DNA double strand breaks more efficiently than their differentiated counterparts (Ferdousi et al., “More Efficient Repair of DNA Double-Strand Breaks in Skeletal Muscle Stem Cells Compared to their Committed Progeny,” Stem Cell Research 13:492-507 (2014), which is hereby incorporated by reference in its entirety), and reduced DNA damage repair could amplify the progressive DNA damage in differentiating myoblasts. In wild-type myofibers, repeated exposure to DNA damaging agents, when combined with DNA damage inhibition, was sufficient to induce cell death to the same extent as observed in untreated Lmna KO cells (FIGS. 14G-14I), demonstrating that accumulating DNA damage is sufficient to induce cell death even in post-mitotic cells such as myofibers.

The role of DNA damage response signaling in post-mitotic muscle function is an area of increasing interest. DNA damage results in rapid activation of DNA damage response pathways, including DNA-PK and ATM, which results in stabilization of p53, one of the primary DNA damage response pathway that can induce cell cycle arrest, senescence, and apoptosis (Williams et al., “P53 in the DNA-Damage-Repair Process,” Cold Spring Harb. Perspect. Med. 6:a026070 (2016) and Kruiswijk et al., “p53 in Survival, Death and Metabolic Health: A Lifeguard with a Licence to Kill,” Nature Reviews Molecular Cell Biology 16:393-405 (2015), which are hereby incorporated by reference in their entirety). The consequences of increased DNA damage response signaling in post-mitotic cells remain poorly characterized, but recent findings point to an intriguing role of DNA damage response pathways in skeletal and cardiac muscle. Increased activity of DNA-PK, one of the major DNA damage sensing pathways in interphase cells (Blackford et al., “ATM, ATR, and DNA-PK: The Trinity at the Heart of the DNA Damage Response,” Mol. Cell 66(6):801-817 (2017), which is hereby incorporated by reference in its entirety), was recently linked to the age-related decline of metabolic, mitochondrial, and physical fitness of skeletal muscle cells (Park et al., “DNA-PK Promotes the Mitochondrial, Metabolic, and Physical Decline that Occurs During Aging,” Cell Metab. 25(5):1135-1146 (2017), which is hereby incorporated by reference in its entirety). Furthermore, cardiac-specific expression of Lmna D300N results in increased DNA damage and activation of p53 in a laminopathy mouse model, and cardiac specific deletion of the Trp53 gene encoding p53 significantly improved the cardiac defects, although only marginally improved overall survival (Chen et al., “DNA Damage Response/TP53 Pathway is Activated and Contributes to the Pathogenesis of Dilated Cardiomyopathy Associated with LMNA (Lamin A/C) Mutations,” Circ. Res. 124:856-873 (2019), which is hereby incorporated by reference in its entirety). Consistent with increased p53 signaling caused by accumulating DNA damage, evidence of caspase-3 activation and a progressive loss in viability in Lmna KO and N195K myofibers was observed (FIGS. 1E-1F). While the mechanisms controlling apoptosis in a post-mitotic tissue such as skeletal muscle are not well understood and remain controversial (Schwartz, L.M., “Skeletal Muscles do not Undergo Apoptosis During Either Atrophy or Programmed Cell Death-Revisiting the Myonuclear Domain Hypothesis,” Frontiers in Physiology 9:1887 (2019), which is hereby incorporated by reference in its entirety), this type of cell death has been observed in myofibers during other muscle wasting conditions (Cheema et al., “Apoptosis and Necrosis Mediate Skeletal Muscle Fiber Loss in Age-Induced Mitochondrial Enzymatic Abnormalities,” Aging Cell 14:1085-1093 (2015), in Lmna E82K mutant (Lu et al., “LMNA E82K Mutation Activates FAS and Mitochondrial Pathways of Apoptosis in Heart Tissue Specific Transgenic Mice,” PLoS One 5:e15167 (2010), which is hereby incorporated by reference in its entirety) and heterozygous Lmna^{+/-} mouse hearts (Wolf et al., “Lamin A/C Haploinsufficiency Causes Dilated Cardiomyopathy and Apoptosis-Triggered Cardiac Conduction System Disease,” Journal of Molecular and Cellular Cardiology 44;293-303 (2008), which is herby incorporated by reference in its entirety), and recently in a cardiac-specific Lmna D300N mouse model (Chen et al., “DNA Damage Response/TP53 Pathway is Activated and Contributes to the Pathogenesis of Dilated Cardiomyopathy Associated with LMNA (Lamin A/C) Mutations,” Circ. Res. 124:856-873 (2019), which is hereby incorporated by reference in its entirety).

Although Lmna mutant myoblasts have mechanically weaker nuclei than wild-type myoblasts, it was found that nuclear envelope damage only arose during myoblast differentiation, when cytoskeletal forces acting on the myonuclei increase. Surprisingly, nuclear damage during in vitro myofiber differentiation was associated with kinesin-1 mediated nuclear movements, independent of actomyosin contractions (FIGS. 21A-21B, 23B-23C, 24B-24D). Kinesin-1 applies localized point forces at the nuclear envelope of skeletal myonuclei, either directly or through microtubules anchored at the nuclear envelope through the LINC complex, to ensure correct nuclear positioning (Roman et al., “Nuclear Positioning in Skeletal Muscle,” Seminars in Cell & Developmental Biology 82:51-56 (2018), which is hereby incorporated by reference in its entirety). These forces acting on the weakened nuclear envelope are likely sufficient to induce nuclear envelope rupture in the Lmna mutant myonuclei, based on recent studies on lamin A/C-depleted cells subjected to precisely controlled tensile forces (Zhang et al.,

“Local, Transient Tensile Stress on the Nuclear Membrane Causes Membrane Rupture,” Mol. Biol. Cell 30:899-906 (2019), which is hereby incorporated by reference in its entirety. Furthermore, mutations in genes encoding lamins A/C and other nuclear envelope proteins linked to muscular dystrophies could disrupt perinuclear cytoskeletal organization, including that of desmins (Nikolova et al., “Defects in Nuclear Structure and function Promote Dilated Cardiomyopathy in Lamin A/C-Deficient Mice,” The Journal of Clinical Investigation 113:357-369 (2004), which is hereby incorporated by reference in its entirety) and the perinuclear microtubule network, which helps to resist cytoplasmic strain and physically protect myonuclei, thereby further promoting nuclear damage in myofibers. At the same time, actomyosin contractility is likely to contribute to nuclear envelope damage in muscle fibers in vivo, which can generate substantially higher forces than in vitro differentiated myofibers, and to nuclear envelope rupture and DNA damage in cardiac myocytes (Cho et al., “Mechanosensing by the Lamina Protects Against Nuclear Rupture, DNA Damage, and Cell-Cycle Arrest,” Dev. Cell 49:920-935 e925 (2019), which is hereby incorporated by reference in its entirety).

While the possibility that altered cytoplasmic signaling and gene regulation pathways contribute to the increased nuclear envelope rupture and DNA damage in Lmna mutant muscle cells cannot be excluded (Chen et al., “DNA Damage Response/TP53 Pathway is Activated and Contributes to the Pathogenesis of Dilated Cardiomyopathy Associated with LMNA (Lamin A/C) Mutations,” Circ. Res. 124:856-873 (2019) and Gonzalo, S., “DNA Damage and Lamins,” Adv. Exp. Med. Biol. 773:377-399 (2014), which are hereby incorporated by reference in their entirety), the data presented herein suggest that the damage is mechanically induced and nucleus-intrinsic, as depletion of Kif5b and disruption of the LINC complex substantially reduced DNA damage in Lmna KO myofibers (FIGS. 8E-8F, 10F-10G, 25E-25F). The results presented herein support a model in which cytoskeletal forces cause chromatin protrusions and nuclear envelope ruptures in mechanically weakened Lmna mutant muscle cell nuclei, triggering DNA damage, which then leads to myofiber dysfunction and death in striated muscle laminopathies (FIGS. 28A-28B). Given the diverse roles of lamins in cellular function, additional mechanisms may further contribute to the pathogenesis of laminopathies, and their specific contribution may depend on the particular mutation and cellular context.

The experimental data presented herein provides novel mechanistic insights into the cellular processes that contribute to the development of striated muscle laminopathies, thereby informing future research efforts to target. Microtubule stabilization is one approach to correct the perturbed force balance and should be further explored as a therapy for striated muscle laminopathies. Paclitaxel was recently reported to improve cardiac conduction defects in Lmna H222P mice by restoring proper connexin 43 localization (Macquart et al., “Microtubule Cytoskeleton Regulates Connexin 43 Localization and Cardiac Conduction in Cardiomyopathy Caused by Mutation in A-type Lamins Gene,” Hum. Mol. Genet. (2018), which is hereby incorporated by reference in its entirety). Here, an additional mechanism by which microtubule stabilization may mitigate damaging forces in striated muscle laminopathies is highlighted. In addition, the results presented herein indicate that DNA damage is increased in mouse models of laminopathies, as well as human patients, and that targeting cell signaling pathways activated by DNA damage, such as DNA-PK (Park et al., “DNA-PK Promotes the Mitochondrial, Metabolic, and Physical Decline that Occurs During Aging,” Cell Metab. 25(5):1135-1146 (2017), which is hereby incorporated by reference in its entirety) and p53 (Chen et al., “DNA Damage Response/TP53 Pathway is Activated and Contributes to the Pathogenesis of Dilated Cardiomyopathy Associated with LMNA (Lamin A/C) Mutations,” Circ. Res. 124:856-873 (2019), which is hereby incorporated by reference in its entirety) may provide a mechanism to ameliorate muscle wasting. Furthermore, the studies presented herein found that hybrid myofibers containing ˜20% Lmna KO and ˜80% wild-type nuclei were indistinguishable from their isogenic wild-type controls, suggesting that delivery of wild-type lamin A/C to a subset of myonuclei by gene delivery or stem cell therapy may be sufficient to rescue myofiber function. Further studies may identify the critical number of wild-type nuclei required to rescue cellular health in laminopathic tissue for therapeutic applications.

Beyond striated muscle laminopathies, insights gained from this work are highly relevant to other biological systems in which nuclei are exposed to physical stress from the cytoskeleton, such as in confined migration of cancer cells (McGregor et al., “Squish and Squeeze-the Nucleus as a Physical Barrier During Migration in Confined Environments,” Current Opinion in Cell Biology 40:32-40 (2016), which is hereby incorporated by reference in its entirety), or intracellular nuclear positioning of polarized epithelial, or neuronal cells (Gundersen et al., “Nuclear Positioning,” Cell 152:1376-1389 (2013), which is hereby incorporated by reference in its entirety). For example, in neuronal cells lacking functional lamin A (Jung et al., “Regulation of Prelamin A but not Lamin C by miR-9, a Brain-Specific microRNA,” PNAS USA 109:E423-431 (2012), which is hereby incorporated by reference in its entirety), defects in B-type lamins (Coffinier et al., “Deficiencies in Lamin B1 and Lamin B2 Cause Neurodevelopmental Defects and Distinct Nuclear Shape Abnormalities in Neurons,” Mol. Biol. Cell 22:4683-4693 (2011), which is hereby incorporated by reference in its entirety) or other nuclear envelope nuclear envelope proteins (e.g., nesprin-1) may make these cells more susceptible to kinesin-mediated nuclear damage leading to neurodevelopmental defects (e.g., cerebellar ataxia). Taken together, these findings highlight a novel mechanism by which weakened myonuclei experience microtubule-mediated nuclear envelope damage, leading to DNA damage and muscle dysfunction, potentially explaining the phenotypes seen in striated muscle laminopathies and a spectrum of other diseases caused by nuclear envelope defects.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of treating a laminopathy affecting skeletal or cardiac muscle in a subject comprising:
selecting a subject who has a laminopathy affecting skeletal or cardiac muscle and
administering, to the selected subject, an inhibitor of a protein associated with a DNA damage response (DDR) pathway to treat the laminopathy affecting skeletal or cardiac muscle in the subject.

2. The method according to claim 1, wherein the selected subject is a human subject.

3. The method according to claim 2, wherein the selected subject is an adult.

4. The method according to claim 2, wherein the selected subject is a neonate or a child.

5. The method according to claim 1, wherein the selected subject has a laminopathy associated with a mutation in one or more genes selected from the group consisting of a Lamin A/C (LMNA), emerin (EMD), nesprin-1 (SYNE1), nesprin-2 (SYNE2), SUN domain-containing protein 1 (SUN1), and SUN domain-containing protein 2 (SUN2).

6. The method according to claim 5, wherein the laminopathy is associated with a mutation in a Lamin A/C (LMNA) gene.

7. The method according to claim 6, wherein the mutation is in the LMNA gene corresponds to a N195K or H222P substitution in SEQ ID NO: 1.

8. The method according to claim 5, wherein the mutation is selected from the group consisting of a deletion, an insertion, a point mutation, a missense mutation, a frame shift mutation, a truncation, a nonsense mutation, and a splice-site mutation.

9. The method according to claim 1, wherein the laminopathy is a striated muscle laminopathy selected from the group consisting of Emery-Dreifuss muscular dystrophy (EDMD), LMNA-related congenital muscular dystrophy (LMNA-CMD), limb-girdle muscular dystrophy type 1B (LGMD1B), dilated cardiomyopathy (DCM), and dilated cardiomyopathy with conduction system defects (DCM-CD).

10. The method according to claim 9, wherein the laminopathy is Emery-Dreifuss muscular dystrophy (EDMD) and the subject has a mutation in the LNMA gene corresponding to a mutation in SEQ ID NO: 1 selected from the group consisting of R25P, R25G, K32x, E33G, E33D, L35V, N39S, N39D, R41S, A43T, Y45C, I46V, D47H, R50S, R50P, I63S, I63N, E65G, L85P, R89L, R89C, L102Q, A130P, R133P, L140P, T150P, L162P, N195D, H222Y, H222P, R225Q, G232E, G232R, L248P, R249W, R249Q, Y259D, K260E, K261x, L263P, Y267H, Y267C, S268P, K270K, L271P, S277P, Q294P, S295P, S303P, S326T, R336Q, M348I, Q355X, L356R, E358K, E361K, M371K, R377L, R377H, R377C, E381A, G382R, R386M, R386K, R401C, D446V, G449D, R453W, L454P, N456K, N456I, N456H, D461Y, W467R, I469T, W498R, L512P, Q517X, W520S, W520G, R527P, T528K, T528R, L530P, R541H, R545C, D596N, G602S, R624H, R644C, and combinations thereof, wherein X indicates a nonsense mutation and wherein x indicates a deletion.

11. The method according to claim 9, wherein the laminopathy is LMNA-related congenital muscular dystrophy (LMNA-CMD) and the subject has a mutation in the LNMA gene corresponding to an amino acid substitution in SEQ ID NO: 1 selected from the group consisting of R28Q, K32E, K32x, L35P, N39Y, N39S, R41C, R50P, R249W, R249Q, L292P, L302P, E358K, L380S, R388C, R453P, R455P, N456D, T528R, R644C, R644H, and combinations thereof, wherein x indicates a deletion.

12. The method according to claim 9, wherein the laminopathy is limb-girdle muscular dystrophy type 1B (LGMD1B) and the subject has a mutation in the LNMA gene corresponding to an amino acid mutation in SEQ ID NO: 1 selected from the group consisting of R25G, T27I, R28Q, E33G, R50S, E65G, R101P, K171K, K208x, R249Q, Y259X, A278T, L292P, S303P, K311R, Q312H, R331P, R377C, R377H, R377L, L379F, R453W, Y481H, Q493X, W498C, L512P, W514R, R527P, T528K, R541S, R541P, D596N, D639G, R644C, and combinations thereof, wherein X indicates a nonsense mutation and wherein x indicates a deletion.

13. The method according to claim 9, wherein the laminopathy is dilated cardiomyopathy (DCM) and the subject has a mutation in the LNMA gene corresponding to an amino acid substitution in SEQ ID NO: 1 selected from the group consisting of R25G, R25P, R25W, R25G, E33G, L35V, N39S, A43T, Y45C, R50S, L59R, R60G, I63N, I63S, E65G, E82K, L85R, R89L, R89C, K97E, R133P, S143P, E161K, L140P, T150P, R189P, R190Q, R190W, D192G, N195K, R196S, E203K, E203G, L215P, H222P, H222Y, Y267C, E317K, A347K, R349L, R399C, R435C, R541C, R541S, S573L, R644C, and combinations thereof.

14. The method according to claim 9, wherein the laminopathy is dilated cardiomyopathy with conduction system defects (DCM-CD) and the subject has a mutation in the LLAMA gene corresponding to an amino acid mutation in SEQ ID NO: 1 selected from the group consisting of Q6X, S22L, R28W, Q36X, Y45C, L52P, E53V, R60G, E82K, L85R, R89L, T91T, L92F, K97E, R101P, R110S, E11X, K117R, K123x, A132P, S143P, E161K, R166P, L183P, E186K, R189W, R190W, R190Q, D192G, D192V, N195K, N195K, E203K, E203G, E203V, I210S, L215P, K219T, K219N, R225X, Q234X, Q246X, Y259H, K260N, Y267H, A278T, E291K, Q312H, E317K, A318T, R321X, R331Q, R335W, R335Q, E347K, M348I, R349L, A350P, Q355X, D357H, D357A, Q358X, R377H, R377L, R388H, R399C, R435C, Q432X, V440M, D461Y, R471, Y481X, Q517X, W520X, G523R, R541S, R541G, R541C, R541H, R541P, S571R, S573L, A617A, G635D, R644C, R654X, and combinations thereof, wherein X indicates a nonsense mutation and wherein x indicates a deletion.

15. The method according to claim 1, wherein the protein associated with a DNA damage response (DDR) pathway is a phosphatidylinositol 3-kinase-related kinase (PIKK).

16. The method according to claim 15, wherein the phosphatidylinositol 3-kinase-related kinase (PIKK) is selected from the group consisting of a DNA-dependent protein kinase (DNA-PK), an ataxia telangiectasia mutated serine-protein kinase (ATM), Suppressor of Morphogenesis in Genitalia-1 (SMG-1), and combinations thereof.

17. The method according to claim 16, wherein the phosphatidylinositol 3-kinase-related kinase (PIKK) is a DNA-dependent protein kinase and the inhibitor selectively targets a DNA-PK catalytic subunit, Ku70, and/or Ku80.

18. The method according to claim 16, wherein the phosphatidylinositol 3-kinase-related kinase (PIKK) is a DNA-PK and the inhibitor is a DNA-PK inhibitor selected from the group consisting of NU7441, NU7026, LY294002, IC86621, IC87102, IC87361, OK-1035, SU1172, NK314, IC486241, vanillin, wortmannin, and GRN163L.

19. The method according to claim 16, wherein the phosphatidylinositol 3-kinase-related kinase (PIKK) is ATM and the inhibitor is an ATM inhibitor selected from the group consisting of KU55933, KU60019, KU559403, CP466722, caffeine, and wortmannin.

20. The method according to claim 16, wherein the phosphatidylinositol 3-kinase-related kinase (PIKK) is ATR and the inhibitor is an ATR inhibitor selected from the group consisting of schisandrin B, NU6027, NVP-BEZ235, VE821, VE822, AZ20, AZD6738, and derivatives thereof.

21. The method according to claim 16, wherein the phosphatidylinositol 3-kinase-related kinase (PIKK) is SMG-1 and the inhibitor is a SMG-1 inhibitor is selected from the group consisting of miR-192, miR-215, LY294002, and wortmannin.

22. The method according to claim 1, wherein the inhibitor is a small molecule, a protein, a peptide, a nucleic acid, an aptamer, or an antibody.

23. The method according to claim 1, wherein the administration of the PIKK inhibitor improves muscle strength, reduces muscle wasting, or reduces muscle cell death.

24. The method according to claim 1, wherein said administering is carried out systemically or locally.

25. The method according to claim 1, wherein said administering is carried out intramuscularly, intravenously, subcutaneously, orally, or intraperitoneally.

26. The method according to claim 1 further comprising:
administering to the subject a microtubule stabilizing agent before, after, or during said administering the inhibitor of a protein associated with a DNA damage response (DDR) pathway.

27. The method according to claim 26, wherein the microtubule stabilizing agent is selected from the group consisting of a taxane, an epothilone, discodermolide, sarcodictyin A, sarcodictyin B, eleutherobin, laulimalide, isolaulimalide, peloruside A, and cyclostreptin.

28. The method according to claim 27, wherein the microtubule stabilizing agent is a taxane selected from the group consisting of paclitaxel, docetaxel, and abraxane.

29. The method according to claim 27, wherein the microtubule stabilizing agent is an epothilone selected from the group consisting of epothilone A, epothilone B, epothilone D, aza-epothilone, BMS-310705, KOS-1584, and sagopilone.

30. The method according to claim 1 further comprising:
administering to the subject a Linker of Nucleoskeleton and Cytoskeleton (LINC) complex disruptor before, after, or during said administering the inhibitor of a protein associated with a DNA damage response (DDR) pathway.

31. The method according to claim 30, wherein the LINC complex disruptor selectively targets a Klarsicht, ANC-1, Syne Homology (KASH)-domain protein or a Sad1p, UNC-84 (SUN)-domain protein.

32. The method accorinding to claim 31, wherein the LINC compex disrupter is a small molecule, a protein, a peptide, a nucleic acid, or an aptamer.

33. The method according to claim 32, wherein the LINC complex disruptor is a dominant negative KASH domain or a dominant negative SUN domain.

34. The method according to claim 32, wherein the LINC complex disruptor is selected from the group consisting of an antibody, Fab fragments, F(ab)₂ fragments, Fab′ fragments, F(ab′)₂ fragments, Fd fragments, Fd′ fragments, and FIT fragments.

35. The method according to claim 32, wherein the LINC complex disruptor is a nucleic acid selected from the group consisting of shRNA, siRNA, and miRNA.

36. A method of treating a laminopathy affecting skeletal or cardiac muscle in a subject comprising:
selecting a subject who has a laminopathy affecting skeletal or cardiac muscle;
administering, to the selected subject, a microtubule stabilizing agent; and
administering, to the selected subject, a Linker of Nucleoskeleton and Cytoskeleton (LINC) complex disruptor before, after, or during said administering the microtubule stabilizing agent to treat the laminopathy affecting skeletal or cardiac muscle in the subject.

37. The method according to claim 36, wherein the selected subject is a human subject.

38. The method according to claim 37, wherein the selected subject is an adult.

39. The method according to claim 37, wherein the selected subject is a neonate or a child.

40. The method according to claim 36, wherein the selected subject has a laminopathy associated with a mutation in one or more genes selected from the group consisting of a Lamin A/C (LMNA), emerin (EMD), nesprin-1 (SYNE1), nesprin-2 (SYNE2), SUN domain-containing protein 1 (SUN1), and SUN domain-containing protein 2 (SUN2).

41. The method according to claim 40, wherein the laminopathy is associated with a mutation in a Lamin A/C (LMNA) gene.

42. The method according to claim 41, wherein the mutation is in the LMNA gene corresponds to a N195K or H222P substitution in SEQ ID NO: 1.

43. The method according to claim 40, wherein the mutation is selected from the group consisting of a deletion, an insertion, a point mutation, a missense mutation, a frame shift mutation, a truncation, a nonsense mutation, and a splice-site mutation.

44. The method according to claim 36, wherein the laminopathy is a striated muscle laminopathy selected from the group consisting of Emery-Dreifuss muscular dystrophy (EDMD), LMNA-related congenital muscular dystrophy (LMNA-CMD), limb-girdle muscular dystrophy type 1B (LGMD1B), dilated cardiomyopathy (DCM), and dilated cardiomyopathy with conduction system defects (DCM-CD).

45. The method according to claim 44, wherein the laminopathy is Emery-Dreifuss muscular dystrophy (EDMD) and the subject has a mutation in the LNMA gene corresponding to a mutation in SEQ ID NO: 1 selected from the group consisting of R25P, R25G,K32x, E33G, E33D, L35V, N39S, N39D, R41S, A43T, Y45C, I46V, D47H, R50S, R50P, I63S, I63N, E65G, L85P, R89L, R89C, L102Q, A130P, R133P, L140P, T150P, L162P, N195D, H222Y, H222P, R225Q, G232E, G232R, L248P, R249W, R249Q, Y259D, K260E, K261x, L263P, Y267H, Y267C, S268P, K270K, L271P, S277P, Q294P, S295P, S303P, S326T, R336Q, M348I, Q355X. L356R, E358K, E361K, M371K, R377L, R377H, R377C, E381A, G382R, R386M, R386K, R401C, D446V, G449D, R453W, L454P, N456K, N456I, N456H, D461Y, W467R, I469T, W498R, L512P, Q517X, W520S, W520G, R527P, T528K, T528R, L530P, R541H, R545C, D596N, G602S, R624H, R644C, and combinations thereof, wherein X indicates a nonsense mutation and wherein x indicates a deletion.

46. The method according to claim 44, wherein the laminopathy is LMNA-related congenital muscular dystrophy (LMNA-CMD) and the subject has a mutation in the LNMA gene corresponding to an amino acid substitution in SEQ ID NO: 1 selected from the group consisting of R28Q, K32E, K32x, L35P, N39Y, N39S, R41C, R50P, R249W, R249Q, L292P, L302P, E358K, L380S, R388C, R453P, R455P, N456D, T528R, R644C, R644H, and combinations thereof, wherein x indicated a deletion.

47. The method according to claim 44, wherein the laminopathy is limb-girdle muscular dystrophy type 1B (LGMD1B) and the subject has a mutation in the LNMA gene corresponding to an amino acid mutation in SEQ ID NO: 1 selected from the group consisting of R25G, T27I, R28Q, E33G, R50S, E65G, R101P, K171K, K208x, R249Q, Y259X, A278T, L292P, S303P, K311R, Q312H, R331P, R377C, R377H, R377L, L379F, R453W, Y481H, Q493X, W498C, L512P, W514R, R527P, T528K, R541S, R541P, D596N, D639G, R644C, and combinations thereof, wherein X indicates a nonsense mutation and wherein x indicates a deletion.

48. The method according to claim 44, wherein the laminopathy is dilated cardiomyopathy (DCM) and the subject has a mutation in the LNMA gene corresponding to an amino acid substitution in SEQ ID NO: 1 selected from the group consisting of R25G, R25P, R25W, R25G, E33G, L35V, N39S, A43T, Y45C, R50S, L59R, R60G, I63N, I63S, E65G, E82K, L85R, R89L, R89C, K97E, R133P, S143P, E161K, L140P, T150P, R189P, R190Q, R190W, D192G, N195K, R196S, E203K, E203G, L215P, H222P, H222Y, Y267C, E317K, A347K, R349L, R399C, R435C, R541C, R541S, S573L, R644C, and combinations thereof.

49. The method according to claim 44, wherein the laminopathy is dilated cardiomyopathy with conduction system defects (DCM-CD) and the subject has a mutation in the LNMA gene corresponding to an amino acid mutation in SEQ ID NO: 1 selected from the group consisting of Q6X, S22L, R28W, Q36X, Y45C, L52P, E53V, R60G, E82K, L85R, R89L, T91T, L92F, K97E, R101P, R110S, E11X, K117R, K123x, A132P, S143P, E161K, R166P, L183P, E186K, R189W, R190W, R190Q, D192G, D192V, N195K, N195K, E203K, E203G, E203V, I210S, L215P, K219T, K219N, R225X, Q234X, Q246X, Y259H, K260N, Y267H, A278T, E291K, Q312H, E317K, A318T, R321X, R331Q, R335W, R335Q, E347K, M348I, R349L, A350P, Q355X, D357H, D357A, Q358X, R377H, R377L, R388H, R399C, R435C, Q432X, V440M, D461Y, R471, Y481X, Q517X, W520X, G523R, R541S, R541G, R541C, R541H, R541P, S571R, S573L, A617A, G635D, R644C, R654X, and combinations thereof, wherein X indicates a nonsense mutation and wherein x indicates a deletion.

50. The method according to claim 36, wherein the microtubule stabilizing agent is a small molecule, a protein, a peptide, a nucleic acid, or an aptamer.

51. The method according to claim 50, wherein the microtubule stabilizing agent is selected from the group consisting of a taxane, an epothilone, discodermolide, sarcodictyin A, sarcodictyin B, eleutherobin, laulimalide, isolaulimalide, peloruside A, and cyclostreptin.

52. The method according to claim 50, wherein the microtubule stabilizing agent is a taxane selected from the group consisting of paclitaxel, docetaxel, and abraxane.

53. The method according to claim 50, wherein the microtubule stabilizing agent is an epothilone selected from the group consisting of epothilone A, epothilone B, epothilone D, aza-epothilone, BMS-310705, KOS-1584, and sagopilone.

54. The method according to claim 36, wherein the LINC complex disruptor selectively targets a Klarsicht, ANC-1, Syne Homology (KASH)-domain protein or a Sad1p, UNC-84 (SUN)-domain protein.

55. The method according to claim 54, wherein the LINC compex disrupter is a small molecule, a protein, a peptide, a nucleic acid, or an aptamer.

56. The method according to claim 55, wherein the LINC complex disruptor is a dominant negative KASH domain or a dominant negative SUN domain.

57. The method according to claim 55, wherein the LINC complex disruptor is selected from the group consisting of an antibody, Fab fragments, F(ab)₂ fragments, Fab′ fragments, F(ab′)₂ fragments, Fd fragments, Fd′ fragments, and Fv fragments.

58. The method according to claim 55, wherein the LINC complex disruptor is a nucleic acid selected from the group consisting of shRNA, siRNA, and miRNA.

59. The method of claim 36, wherein the administration of the microbtubule stabilizing agent and the LINC complex disruptor improves muscle strength, reduces muscle wasting, or reduces muscle cell death.

60. The method according to claim 36, wherein said administering steps are carried out systemically or locally.

61. The method according to claim 36, wherein said administering steps are carried out intramuscularly, intravenously, subcutaneously, orally, or intraperitoneally.

62. A pharmaceutical composition comprising:
an inhibitor of a protein associated with a DNA damage response (DDR) pathway and
a microtubule stabilizing agent.

63. The pharmaceutical composition according to claim 62, wherein the protein associated with a DNA damage response (DDR) pathway is a phosphatidylinositol 3-kinase-related kinase (PIKK).

64. The pharmaceutical composition according to claim 63, wherein the phosphatidylinositol 3-kinase-related kinase (PIKK) is selected from the group consisting of a DNA-dependent protein kinase (DNA-PK), an ataxia telangiectasia mutated serine-protein kinase (ATM), Suppressor of Morphogenesis in Genitalia-1 (SMG-1), and combinations thereof.

65. The pharmaceutical composition according to claim 64, wherein the phosphatidylinositol 3-kinase-related kinase (PIKK) is a DNA-dependent protein kinase and the inhibitor selectively targets a DNA-PK catalytic subunit, Ku70, and/or Ku80.

66. The pharmaceutical composition according to claim 62, wherein the microtubule stabilizing agent is selected from the group consisting of a taxane, an epothilone, discodermolide, sarcodictyin A, sarcodictyin B, eleutherobin, laulimalide, isolaulimalide, peloruside A, and cyclostreptin.

67. The pharmaceutical composition according to claim 66, wherein the microtubule stabilizing agent is a taxane selected from the group consisting of paclitaxel, docetaxel, and abraxane.

68. The pharmaceutical composition according to claim 66, wherein the microtubule stabilizing agent is an epothilone selected from the group consisting of epothilone A, epothilone B, epothilone D, aza-epothilone, BMS-310705, KOS-1584, and sagopilone.

69. The pharmaceutical composition according to claim 62 further comprising:
a Linker of Nucleoskeleton and Cytoskeleton (LINC) complex disruptor.

70. The pharmaceutical composition according to claim 69, wherein the LINC complex disruptor selectively targets a Klarsicht, ANC-1, Syne Homology (KASH)-domain protein or a Sad1p, UNC-84 (SUN)-domain protein.

71. The pharmaceutical composition according to claim 70, wherein the LINC compex disruptor is a small molecule, a protein, a peptide, a nucleic acid, or an aptamer.

72. The pharmaceutical composition according to claim 71, wherein the LINC complex disruptor is a dominant negative KASH domain or a dominant negative SUN domain.

73. The pharmaceutical composition according to claim 71, wherein the LINC complex disruptor is selected from the group consisting of an antibody, Fab fragments, F(ab)₂ fragments, Fab′ fragments, F(ab′)₂ fragments, Fd fragments, Fd′ fragments, and Fv fragments.

74. The pharmaceutical composition according to claim 71, wherein the LINC complex disruptor is a nucleic acid selected from the group consisting of shRNA, siRNA, and miRNA.

75. A pharmaceutical composition comprising:
a Linker of Nucleoskeleton and Cytoskeleton (LINC) complex disruptor and
a microtubule stabilizing agent.

76. The pharmaceutical composition according to claim 75, wherein the LINC complex disruptor selectively targets a Klarsicht, ANC-1, Syne Homology (KASH)-domain protein or a Sad1p, UNC-84 (SUN)-domain protein.

77. The pharmaceutical composition according to claim 76, wherein the LINC compex disrupter is a small molecule, a protein, a peptide, a nucleic acid, or an aptamer.

78. The pharmaceutical composition according to claim 77, wherein the LINC complex disruptor is a dominant negative KASH domain or a dominant negative SUN domain.

79. The pharmaceutical composition according to claim 77, wherein the LINC complex disruptor is selected from the group consisting of an antibody, Fab fragments, F(ab)₂ fragments, Fab′ fragments, F(ab′)₂ fragments, Fd fragments, Fd′ fragments, and Fv fragments.

80. The pharmaceutical composition according to claim 77, wherein the LINC complex disruptor is a nucleic acid selected from the group consisting of shRNA, siRNA, and miRNA.

81. The pharmaceutical composition according to claim 75, wherein the microtubule stabilizing agent is selected from the group consisting of a taxane, an epothilone, discodermolide, sarcodictyin A, sarcodictyin B, eleutherobin, laulimalide, isolaulimalide, peloruside A, and cyclostreptin.

82. The pharmaceutical composition according to claim 81, wherein the microtubule stabilizing agent is a taxane selected from the group consisting of paclitaxel, docetaxel, and abraxane.

83. The pharmaceutical composition according to claim 81, wherein the microtubule stabilizing agent is an epothilone selected from the group consisting of epothilone A, epothilone B, epothilone D, aza-epothilone, BMS-310705, KOS-1584, and sagopilone.
